(19) United States
(12) Patent Application Publication
(10) Pub. No.: US 2009/0023666 A1
(43) Pub. Date: Jan. 22, 2009

(54) Modulators of Hypoxia Inducible Factor-1 and Related Uses

(75) Inventors: Gregory Gardiner, Stonington, CT (US); Mehran Khodadoust, Brookline, MA (US); Hans-Jurgen Hess, Old Lyme, CT (US); Saijat Hussoin, Lexington, MA (US); Reimar Bruening, Fremont, CA (US)

Correspondence Address: CLARK & ELBING LLP, 101 FEDERAL STREET, BOSTON, MA, 02110, US

(73) Assignee: BTG plc, London (GB)

(21) Appl. No.: 12/087,459

(22) PCT Filed: Jan. 9, 2007
(86) PCT No.: PCT/US2007/000340

§ 371 Date: Sep. 5, 2008

Related U.S. Application Data
(60) Provisional application No. 60/757,814, filed on Jan. 9, 2006.

(51) Int. Cl. A61K 31/7048; C07H 5/06; C07J 17/00; A61P 35/00; C12N 5/06; A61K 31/585; C07H 17/04

(52) U.S. Cl. 514/26; 536/29.1; 514/42; 536/6; 540/104; 514/175; 435/375

(57) ABSTRACT

The invention features compounds of formulas I or II: and pharmaceutically acceptable salts and prodrugs thereof, as well methods for modulating the effects of local and systemic hypoxic events using the compounds.

### BACKGROUND OF THE INVENTION

The invention relates to cardiolide and bufadienolide compounds and their use for modulating the effects of local and systemic hypoxic events.

Hypoxia provokes a wide range of physiological and cellular responses in humans and other mammals. The effects of hypoxia vary qualitatively depending on the length of time over which hypoxic conditions are maintained. Acute hypoxia is characterized by increased respiratory ventilation, but after 3-5 minutes, ventilation declines. Individuals exposed to chronic hypoxic conditions undergo a suite of responses including decreased heart rate and increased blood pressure. Metabolically, hypoxia causes decreased glucose oxidation with a shift from oxidative phosphorylation to glycolysis. Glycolysis provides a poorer yield of energy from carbohydrates, and oxidation of fatty acids is greatly reduced. Perhaps for these reasons, hypoxia also triggers increased consumption of carbohydrates. Hypoxia stimulates production of erythropoietin, which in turn leads to an increase in the red blood cell count.

Hypoxia may occur at the level of the whole organism, as, for example, when ventilation is interrupted or when oxygen availability is low. Hypoxia may also occur at a local level essentially any time oxygen consumption outpaces the supply from the bloodstream. Ischemic events are severe forms of local hypoxia that lead to cell death. Recent discoveries relating to the HIF-1 transcription factor have provided considerable insight into the local, cellular response to hypoxia, but our understanding of how the overall physiological response is regulated, and how the systemic and local responses might interact is more limited.

HIF-1 is a transcription factor and is critical to cellular survival in hypoxic conditions, both in cancer and cardiac cells. HIF-1 is composed of the growth factor-regulated subunit HIF-1α, and the constitutively expressed HIF-1β subunit (aryl-hydrocarbon receptor nuclear translocator, ARNT), both of which belong to the basic helix-loop-helix (bHLH)-PAS (PER, ARNT, SIM) protein family. In the human genome, three isoforms of the subunit of the transcription factor HIF have been identified: HIF-1, HIF-2 (also referred to as EPAS-1, MOP2, HLF, and HRF), and HIF-3 (of which HIF-32 also referred to as IPAS, inhibitory PAS domain).

Under normoxic conditions, HIF-1α is targeted for ubiquitinylation by pVHL and is rapidly degraded by the proteasome. This is triggered through post-translational HIF-1α hydroxylation on specific proline residues (proline 402 and 564 in human HIF-1α protein) within the oxygen dependent degradation domain (ODDD), by specific HIF-prolyl hydroxylases (HPH1-3 also referred to as PHD1-3) in the presence of iron, oxygen, and 2-oxoglutarate. The hydroxylated protein is then recognized by pVHL, which functions as an E3 ubiquitin ligase. The interaction between HIF-1α and pVHL is rer accelerated by acetylation of lysine residue 532 through an N-acetyltransferase (ARD1). Concurrently, hydroxylation of the asparagine residue 803 within the C-TAD also occurs by an asparaginyl hydroxylase (also referred to as F1H-1), which by its turn does not allow the coactivator p300/CBP to bind to HIF-1 subunit. In hypoxic conditions, HIF-1α remains not hydroxylated and does not interact with pVHL and CBP/p300.

Following hypoxic stabilization, HIF-1α translocates to the nucleus where it heterodimerizes with HIF-1β. The resulting activated HIF-1 drives the transcription of over 60 genes important for adaptation and survival under hypoxia including glycolytic enzymes, glucose transporters Glut-1 and Glut-3, endothelin-1 (ET-1), VEGF (vascular endothelial growth factor), tyrosine hydroxylase, transferrin, and erythropoietin (Brahimi-Horn et al., Trends Cell Biol. 11:S32-S36, 2001; Beasley et al., Cancer Res. 62:2493-2497, 2002; Fukuda et al., J. Biol. Chem. 277: 38205-38211, 2002; and Maxwell and Ratcliffe, Semin. Cell Dev. Biol. 13:29-37, 2002).

While HIF-1 is now understood to be the principal mediator of local, or cellular, responses to hypoxia, no global regulator of hypoxia has yet been recognized. It is an object of the invention to identify regulators of hypoxia, and further, to provide uses for such regulators.

Certain compounds are disclosed in Int. Immunopharmac. (2001), 1(1), 119-134 (Terness et al.); Justus Liebigs Annalen der Chemie (1971), 753, 116-34 Goerlich et al.), Naunyn-Schmiedeberg's Arch. Pharmacol., 329 (4), 1985, 414-426 (Schönfeld et al.), J. Pharmacol. Exp. Ther. (1980), 215(1), 198-204 (Cook et al.), J. Cardiovasc Pharmacol. (1979), 1(5), 551-9 (Cook et al.) and J. Pharmacol. Exp. Ther. (1978), 204(1), 141-8 (Caldwell et al.), and in WO 2006/002381-A1 (WARF), WO 2006/120472-A2 (Guy's and St Thomas' NHS Foundation Trust) and co-pending application No. PCT/U.S. 06/030224 filed Aug. 1, 2006.

### SUMMARY OF THE INVENTION

The present invention is based on the discovery of compounds that modulate the effects of local and systemic hypoxic events. Dysregulation (e.g. excessive or insufficient signaling) of the HIF-steroid signaling pathway can contribute, in a downstream fashion, to a wide variety of disorders including, without limitation, cancer, macular degeneration, hyperglycemia, metabolic syndrome (e.g. Syndrome X), cataracts, hypertension, autoimmune disorders, anxiety, depression, insomnia, chronic fatigue, epilepsy, and symptoms associated with irregular angiogenesis. The compounds of the invention, which are modulators (e.g. agonists and antagonists) of the HIF-steroid signaling pathway, can be used to treat these disorders.

Accordingly, in a first aspect the invention features a compound of formulas I or II:

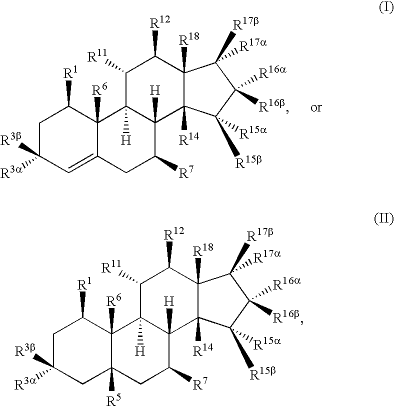

or a pharmaceutically acceptable salt or prodrug thereof. In formulas I and II each of R¹, R⁵, R⁷, R¹¹, and R¹² is, independently, H; OH, OR^{1A}, or OC(O)R^{1A}, where R^{1A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; each of R^{3α} and R^{3β} is, independently, H, OC(O)NHR^{3C}, OC(O)NR^{3D}R^{3E}, NH₂, NHR^{3F}, NR^{3G}R^{3H}, NHC(O)R^{3I}, NHC(O)OR^{3J}, NR^{3K}C(O)OR^{3L}, or NH-Sac, where each of R^{3C}, R^{3D}, R^{3E}, R^{3F}, R^{3G}, R^{3H}, R^{3I}, R^{3J}, R^{3K}, and R^{3L} is, independently, C₁₋₇ alkyl, C₂₋₇ alkenyl, C₂₋₇ alkynyl, C₂₋₆ heterocyclyl, C₆₋₂ aryl, C₇₋₁₄ alkaryl, C₃₋₁₀ alkheterocyclyl, or C₁₋₇ heteroalkyl, and Sac is a saccharide, or R^{3α} and R^{3β} together are ═NNR^{3M}R^{3N}, or —NOR^{3P}, wherein each of R^{3M}, R^{3N} and R^{3P} is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, and with the proviso that at least one of R^{3α} and R³β is not H; R⁶ is CH₃, CH₂OR^{6A}, or CH₂OCOR^{6A}, where C^{6A} is H, C₁₋₇ alkyl, C₂₋₇ alkenyl, C₂₋₇ allynyl, C₂₋₆ heterocyclyl, C₆₋₁₂ aryl, C₇₋₁₄ alkaryl, C₃₋₁₀ alkheterocyclyl, or C₁₋₇ heteroalkyl; R¹⁴ is OH, Cl, OR^{14A}, or OC(O)R^{14A}, where R^{14A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R¹⁴, R^{15β}, and the carbons they are bonded to together represent an epoxide; each of R^{15α} and R^{15β} is, independently, H, OH, OR^{15A}, or OC(O)R^{15A}, where R^{15A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{15α} and R^{15β} together are ═O; each of R^{16α}, and R^{16β} is, independently, H, OH, OR^{16A}, or OC(O)R^{16A}, where R^{16A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{16α} and R^{16β} together are ═O; R^{17β} is

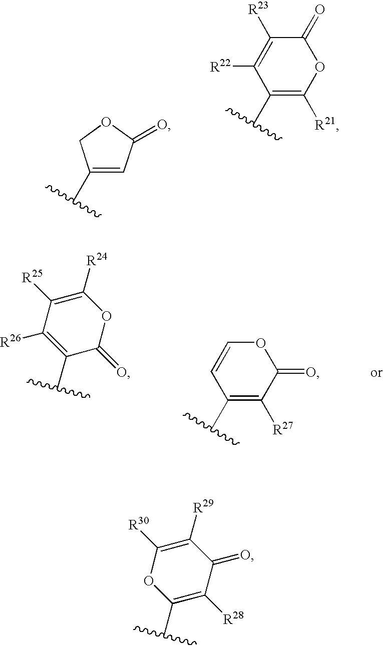

where each of R²¹, R²², R²³, R²⁴, R²⁵, R²⁶, R²⁷, R²⁸, R²⁹, and R³⁰ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R^{17α} is H or OH; and R¹⁸ is CH₃, CH₂OR^{18A}, or CH₂OCOR^{18A}, where R^{18A} is H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

In an embodiment of the above aspect, each of R¹, R^{3α}, R⁵, R⁷, R¹¹, R¹², R^{15α}, R^{15β}, R^{16α}, and R^{16β} is H; and each of R⁶ and R¹⁸ is CH₃; R¹⁴ is OH; R^{3β} is OC(O)NHR^{3C}, OC(O)NR^{3D}R^{3E}, NH₂, NHR^{3F}, NR^{3G}R^{3H}, NHC(O)R^{3I}, NHC(O)OR^{3J}, NR^{3K}C(O)OR^{3L} or NH-Sac.

Desirably, R^{3β} is NH-Sac and Sac is described by the formula:

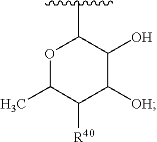

wherein R⁴⁰ is F, Cl, CF₃, OH, NH₂, NHR^{40A}, NR^{40B}R^{40C}, NHC(O)R^{40D}, NHC(S)R^{40E}, NHC(O)OR^{40F}, NHC(S)OR^{40G}, NHC(O)NHR^{40H}, NHC(S)NHR^{40I}, NHC(O)SR^{40J}, NHC(S)SR^{40K}, or NHS(O)₂R^{40L}; and each of R^{40A}, R^{40B}, R^{40C}, R^{40D}, R^{40E}, R^{40F}, R^{40G}, R^{40H}, R^{40I}, R^{40J}, R^{40K}, and R^{40L} is, independently, C₁₋₇ alkyl, C₂₋₇ alkenyl, C₂₋₇ alkynyl, C₂₋₄ heterocyclyl, C₆₋₁₂ aryl, C₇₋₁₄ alkaryl, C₃₋₁₀ alkheterocyclyl, or C₁₋₇ heteroalkyl, or R^{40B} and R^{40C} combine to form a C₂₋₄ heterocyclyl containing at least one nitrogen atom. An exemplary compound of formula I is

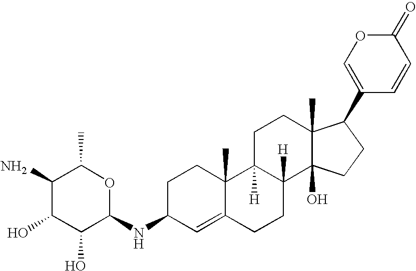

Other preferred values for R^{3α} and R^{3β} are one group being H and the other OC(O)NHR^{3C} where R^{3C} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{3α} and R^{3β} together are ═NOR^{3P}, wherein R^{3P} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

In another aspect, the invention features a compound of formula III:

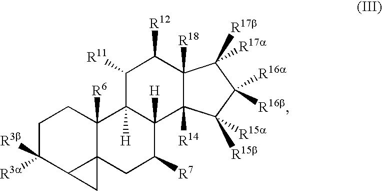

or a pharmaceutically acceptable salt or prodrug thereof. In formula III each of R¹, R⁵, R⁷, R¹¹, and R¹² is, independently, H; OH, OR^{1A}, or OC(O)R^{1A}, where R^{1A} is C₁₋₇ alkyl, C₂₋₇ alkenyl, C₂₋₇ alkynyl, C₂₋₄ heterocyclyl, C₆₋₁₂ aryl, C₇₋₁₄ alkaryl, C₃₋₁₀ alkheterocyclyl, or C₁₋₇ heteroalkyl; each of R^{3α} and R^{3β} is, independently, H, OH, OR^{3A}, OC(O)^{3B}, OC(O)NHR^{3C}, OC(O)NR^{3D}R^{3E}, O-Sac, NH₂, NHR^{3F}, NR^{3G}R^{3H}, NHC(O)R^{3I}, NHC(O)OR^{3J}, NR^{3K}C(O)PR^{3L}, or NH-Sac, where each of R^{3A}, R^{3B}, R^{3C}, R^{3D}, R^{3E}, R^{3F}, R^{3G}, R^{3H}, R^{3I}, R^{3J}, R^{3K}, and R^{3L} is, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, and Sac is a saccharide, or R^{3α} and R³β together are ═O, ═NNR^{3M}R^{3N}, or ═NOR^{3P}, wherein each of R^{3M}, R^{3N} and R^{3P} is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, and with the proviso that at least one of R^{3α} and R^{3β} is not H; R⁶ is CH₃, CH₂OR^{6A}, or CH₂OCOR^{6A} H where R^{6A} is H, C₁₋₇ alkyl, C₂₋₇ alkenyl, C₂₋₇ alkynyl, C₂₋₄ heterocyclyl, C₆₋₁₂ aryl, C₇₋₁₄ alkaryl, C₃₋₁₀ alkheterocyclyl, or C₁₋₇ heteroalkyl; R¹⁴ is OH, Cl, OR^{14A}, or OC(O)R^{14A}, where R^{14A} is C₁₋₇ alkyl, C₂₋₇ alkenyl, C₂₋₇ alkynyl, C₂₋₄ heterocyclyl, C₆₋₁₂ aryl, C₇₋₁₄ alkaryl, C₃₋₁₀ alkheterocyclyl, or C₁₋₇ heteroalkyl, or R¹⁴, R^{15β}, and the carbons they are bonded to together represent an epoxide; each of R^{15α} and R^{15β} is, independently, H, OH, OR^{15A}, or OC(O)R^{5A}, where R^{15A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{15α} and R^{15β} together are ═O; each of R^{16α} and R^{16β} is, independently, H, OH, OR^{16A}, or OC(O)R^{16A}, where R^{16A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{16α} and R^{16β} together are ═O; R^{17β} is

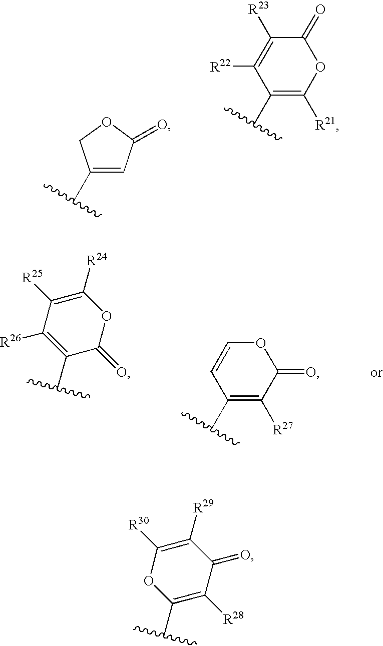

where each of R²¹, R²², R²³, R²⁴, R²⁵, R²⁶, R²⁷, R²⁸, R²⁹, and R³⁰ is, independently, H, C₁₋₇ alkyl, C₂₋₇ alkenyl, C₂₋₇ alkynyl, C₂₋₆ heterocyclyl, C₆₋₂ aryl, C₇₋₁₄ alkaryl, C₃₋₁₀ alkheterocyclyl, or C₁₋₇ heteroalkyl; R^{17α} is H or OH; and R¹⁸ is CH₃, CH₂OR^{18A}, or CH₂OCOR^{18A}, where R^{18A} is H, C₁₋₇ alkyl, C₂₋₇ alkenyl, C₂₋₇ alkynyl, C₂₋₄ heterocyclyl, C₆₋₁₂ aryl, C₇₋₁₄ alkaryl, C₃₋₁₀ alkheterocyclyl, or C₁₋₇ heteroalkyl.

In an embodiment of the above aspect, each of R¹, R^{3α}, R⁷, R¹¹, R¹², R^{15α}, R^{15β}, R^{16α}, and R^{16β} is H; and each of R⁶ and R¹⁸ is CH₃; R¹⁴ is OH; R^{3P} is OC(O)NHR^{3C}, OC(O)NR^{3D}R^{3E}, O-Sac, NH₂, NHR^{3F}, NR^{3G}R^{3H}, NHC(O)R^{3I}, NHC(O)OR^{3J}, NR^{3K}C(O)OR^{3L}, or NH-Sac.

In an embodiment of the above aspect, R^{3β} is O-Sac, or NH-Sac; Sac is described by the formula:

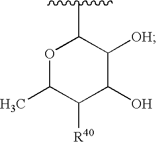

wherein R⁴⁰ is F, Cl, CF₃, OH, NH₂, NHR^{40A}, NR^{40B}R^{40C}, NHC(O)R^{40D}, NHC(S)R^{40E}, NHC(O)OR^{40F}, NHC(S)OR^{41G}, NHC(O)NHR^{40H}, NHC(S)NHR^{40I}, NHC(O)SR^{40J}, NHC(S)SR^{40K}, or NHS(O)₂R^{40L}; and each of R^{40A}, R^{40B}, R^{40C}, R^{40D}, R^{40E}, R^{40F}, R^{40G}, R^{40H}, R^{40I}, R^{40J}, R^{40K}, and R^{40L} is, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{40B} and R^{40C} combine to form a C₂₋₄ heterocyclyl containing at least one nitrogen atom.

In a further aspect, the invention features a compound of formula IV:

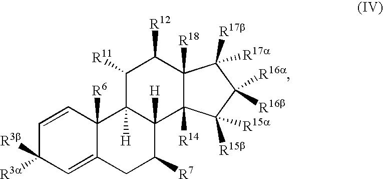

or a pharmaceutically acceptable salt or prodrug thereof. In formula IV each of R¹, R⁵, R⁷, R¹¹, and R¹² is, independently, H; OH, OR^{1A}, or OC(O)R^{1A}, where R^{1A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, each of R^{3α} and R^{3β} is, independently, H, OC(O)NHRC, OC(O)NR^{3D}R^{3E}, NH₂, NHR^{3F}, NR^{3G}R^{3H}, NHC(O)R^{3I}, NHC(O)OR^{3J}, NR^{3K}C(O)OR^{3L}, or NH-Sac, where each of R^{3C}, R^{3D}, R^{3E}, R^{3F}, R^{3G}, R^{3H}, R^{3I}, R^{3J}, R^{3K}, and R^{3L} is, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, and Sac is a saccharide, or R^{3α} and R^{3β} together are ═NNR^{3M}R^{3N}, or ═NOR^{3P}, wherein each of R^{3M}, R^{3N} and R^{3P} is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, and with the proviso that at least one of R^{3α} and R^{3β} is not H; R⁶ is CH₃, CH₂OR^{6A}, or CH₂OCOR^{6A}, where R^{6A} is H, C₁₋₇ alkyl, C₂₋₇ alkenyl, C₂₋₇ alkynyl, C₂₋₄ heterocyclyl, C₆₋₁₂ aryl, C₇₋₁₄ alkaryl, C₃₋₁₀ alkheterocyclyl, or C₁₋₇ heteroalkyl; R¹⁴ is OH, Cl, OR^{14A}, or OC(O)R^{14A}, where R^{14A} is C₁₋₇ alkyl, C₂₋₇ alkenyl, C₂₋₇ alkynyl, C₂₋₄ heterocyclyl, C₆₋₁₂ aryl, C₇₋₁₄ alkaryl, C₃₋₁₀ alkheterocyclyl, or C₁₋₇ heteroalkyl, or R¹⁴, R^{15β}, and the carbons they are bonded to together represent an epoxide; each of R^{15α} and R^{15β} is, independently, H, OH, OR^{15A}, or OC(O)R^{15A}, where R^{15A} is Cl₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{15α} and R^{15β} together are ═O; each of R¹⁶, and R^{16β} is, independently, H, OH, OR^{16A}, or OC(O)R^{16A}, where R^{16A} is C₁₋₇ alkyl, C₂₋₇ alkenyl, C₂₋₇ alkynyl, C₂₋₄ heterocyclyl, C₆₋₁₂ aryl, C₇₋₁₄ alkaryl, C₃₋₁₀ alkheterocyclyl, or C₁₋₇ heteroalkyl, or R^{16α} and R^{16β} together are ═O; R^{17β} is

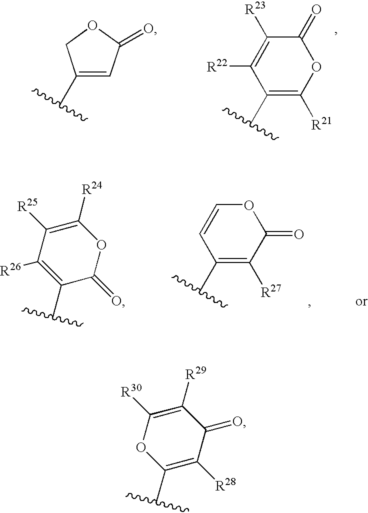

where each of R²¹, R²², R²³, R²⁴, R²⁵, R²⁶, R²⁷, R²⁸, R²⁹, and R³⁰ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R¹⁷ is H or OH; and R¹⁸ is CH₃, CH₂OR^{18A}, or CH₂OCOR^{18A}, where R^{18A} is H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

In an embodiment of the above aspect, each of R¹, R^{3α}, R⁷, R¹¹, R¹², R^{15α}, R^{15β}, R^{16α}, and R^{16β} is H; and each of R⁶ and R¹⁸ is CH₃; R¹⁴ is OH; R³ is OH, OR^{3A}, OC(O)R^{3B}, OC(O)NHR^{3C}, OC(O)NR^{3D}R^{3E}, O-Sac, NH₂, NHR^{3F}, NRGR^{3G}R^{3H}, NHC(O)R^{3I}, NHC(O)OR^{3J}, NR^{3K}C(O)OR^{3L}, or NH-Sac.

Desirably, R^{3β} is NH-Sac and Sac is described by the formula:

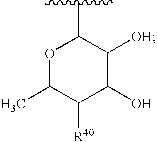

wherein R⁴⁰ is F, Cl, CF₃, OH, NH₂, NHR^{40A}, NR^{40B}R^{40C}, NHC(O)R^{40D}, NHC(S)R^{40E}, NHC(O)OR^{40F}, NHC(S)OR^{40G}, NHC(O)NHR^{40H}, NHC(S)NHR^{40I}, NHC(O)SR^{40J}, NHC(S)SR^{40K}, or NHS(O)₂R^{40L}; and each of R^{40A}, R^{40B}, R^{40C}, R^{40D}, R^{40E}, R^{40F}, R^{40G}, R^{40H}, R^{40I}, R^{40J}, R^{40K}, and R^{40L} is, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{40B} and R^{40C} combine to form a C₂₋₆ heterocyclyl containing at least one nitrogen atom.

In still another aspect, the invention features a compound of formulas Ia or IIa:

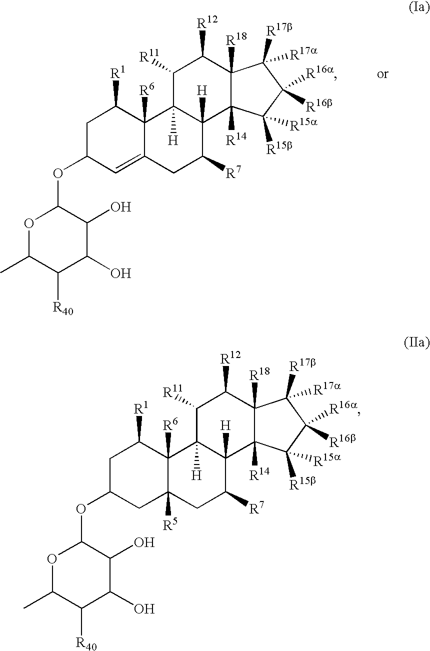

or a pharmaceutically acceptable salt or prodrug thereof. In formulas Ia and IIa each of R¹, R⁵, R⁷, R¹¹, and R¹² is, independently, H; OH, OR^{1A}, or OC(O)R^{1A}, where R^{1A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R⁶ is CH₃, CH₂OR^{6A}, or CH₂OCOR^{6A}, where R^{6A} is H, C₁₋₇ alkyl, C₂₋₇ alkenyl, C₂₋₇ alkynyl, C₂₋₆ heterocyclyl, C₆₋₂ aryl, C₇₋₁₄ alkaryl, C₃₋₁₀ alkheterocyclyl, or C₁₋₇ heteroalkyl; R¹⁴ is OH, Cl, OR^{4A}, or OC(O)R^{14A}, where R^{14A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R¹⁴, R¹⁵, and the carbons they are bonded to together represent an epoxide; each of R^{15α} and R^{15β} is, independently, H, OH, OR^{15A}, or OC(O)R^{15A}, where R^{15A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{15α} and R^{15β} together are ═O; each of R^{16α} and R^{16β} is, independently, H, OH, OR^{16A}, or OC(O)R^{16A}, where R^{16A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{16α} and R^{16β} together are ═O; R^{17β} is

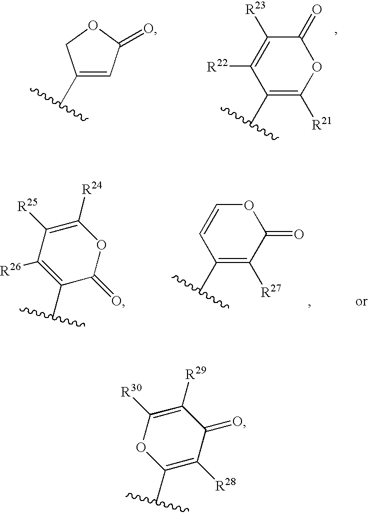

where each of R²¹, R²², R²³, R²⁴, R²⁵, R²⁶, R²⁷, R²⁸, R²⁹, and R³⁰ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R¹⁷ is H or OH; R¹⁸ is CH₃, CH₂OR_{18A}, or CH₂OCOR^{18A}, where R^{18A} is H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; and R⁴⁰ is F, Cl, CF₃, NH₂, NHR^{40A}, NR^{40B}R^{40C}, NHC(O)^{40D}, NHC(S)R^{40E}, NHC(O)OR^{40F}, NHC(S)OR^{40G}, NHC(O)NHR^{40H}, NHC(S)NHR^{40I}, NHC(O)SR^{40J}, NHC(S)SR^{40K}, or NHS(O)₂R^{40L}, and where each of R^{40A}, R^{40B}, R^{40C}, R^{40D}, R^{40E}, R^{40F}, R^{40H}, R^{40H}, R^{40I}, R^{40J}, R^{40K}, and R^{40L} is, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; or R^{40B} and R^{40C} combine to form a C₂₋₆ heterocyclyl containing at least one nitrogen atom. An exemplary compound of formula Ia is

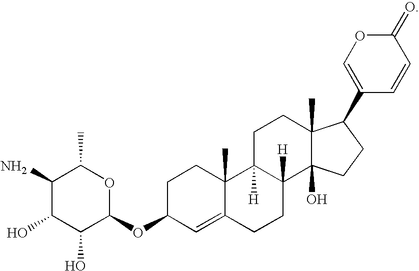

In yet another aspect, the invention features a compound of formula IVa:

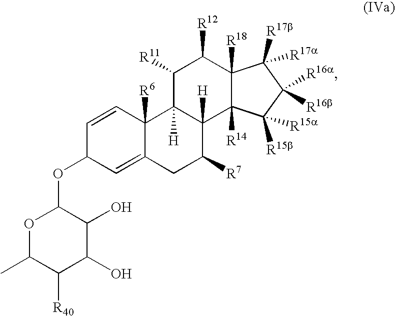

or a pharmaceutically acceptable salt or prodrug thereof. In formula IVa each of R¹, R⁵, R⁷, R¹¹, and R¹² is, independently, H; OH, OR^{1A}, or OC(O)R^{1A}, where R^{1A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R⁶ is CH₃, CH₂OR^{6A}, or CH₂OCOR^{6A}, where R^{6A} is H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R¹⁴ is OH, Cl, OR^{14A}, or OC(O)R^{14A}, where R^{14A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R¹⁴, R^{15β}, and the carbons they are bonded to together represent an epoxide; each of R^{15α} and R^{15β} is, independently, H, OH, OR^{15A}, or OC(O)R^{15A}, where R^{15A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{15α} and R^{15β} together are ═O; each of R^{16α} and R^{16β} is, independently, H, OH, OR^{16A}, or OC(O)R^{16A}, where R^{16A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{16α} and R^{16β} together are ═O; R^{17β} is

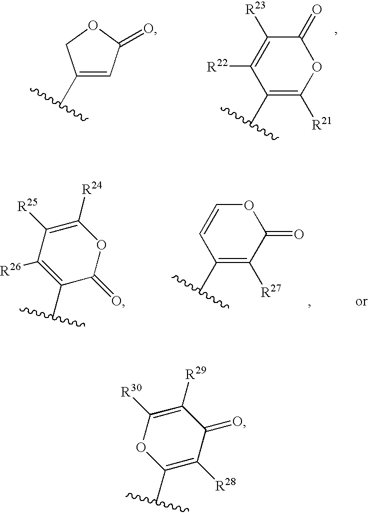

where each of R²¹, R²², R²³, R²⁴, R²⁵, R²⁶, R²⁷, R²⁸, R²⁹, and R³⁰ is, independently, H, C₁₋₇ alkyl, C₂₋₇ alkenyl, C₂₋₇ alkynyl, C₂₋₄ heterocyclyl, C₆₋₁₂ aryl, C₇₋₁₄ alkaryl, C₃₋₁₀ alkheterocyclyl, or C₁₋₇ heteroalkyl; R^{17α} is H or OH; R¹⁸ is CH₃, CH₂OR^{18A}, or CH₂OCOR^{18A}, where R^{18A} is H, C₁₋₇ alkyl, C₁₋₇ alkenyl, C₂₋₇ alkynyl, C₂₋₄ heterocyclyl, C₆₋₁₂ aryl, C₇₋₁₄ alkaryl, C₃₋₁₀ alkheterocyclyl, or C₁₋₇ heteroalkyl; and R⁴⁰ is F, Cl, CF₃, NH₂, NHR^{40A}, NR^{40B}R^{40C} NHC(O)R^{41D}, NHC(S)R^{40E}, NHC(O)OR^{40F}, NHC(S)OR^{40G}, NHC(O)NHR^{40H}, NHC(S)NHR^{40I}, NHC(O)SR^{40J}, NHC(S)SR^{40K}, or NHS(O)₂R^{40J}, and where each of R^{40A}, R^{40B}, R^{40C}, R^{40D}, R^{40E}, R^{40F}, R^{40G}, R^{40H}, R^{40I}, R^{40J}, R^{40K}, and R^{40L} is, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; or R^{40B} and R^{40C} combine to form a C₂₋₆ heterocyclyl containing at least one nitrogen atom.

In another aspect, the invention also features a compound of formulas Ib or IIb:

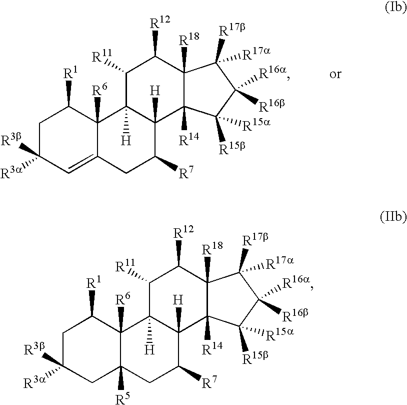

or a pharmaceutically acceptable salt or prodrug thereof. In formulas Ib and IIb each of R¹, R⁵, R⁷, R¹¹, and R¹² is, independently, H; OH, OR^{1A}, or OC(O)R^{1A}, where R^{1A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; each of R^{3α} and R³ is, independently, H, OR^{3A} or OC(O)R^{3B} and each of R^{3A} and R^{3B} is, independently, C₂₋₆ heterocyclyl, C₆₋₁₂ aryl, C₇₋₁₄ alkaryl, C₃₋₁₀ alkheterocyclyl, or C₁₋₇ heteroalkyl, with the proviso that at least one of R^{3α} and R^{3β} is not H; R⁶ is CH₃, CH₂OR^{6A}, or CH₂OCOR^{6A}, where R^{6A} is H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R¹⁴ is OH, Cl, OR^{14A}, or OC(O)R^{14A}, where R^{14A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R⁴, R^{15β} and the carbons they are bonded to together represent an epoxide; each of R^{15α} and R¹⁵ is, independently, H, OH, OR^{15A}, or OC(O)R^{15A}, where R^{15A} is C₁₋₇ alkyl, C₂₋₇ alkenyl, C₂₋₇ alkynyl, C₂₋₆ heterocyclyl, C₆₋₂ aryl, C₇₋₁₄ alkaryl, C₃₋₁₀ alkheterocyclyl, or C₁₋₇ heteroalkyl, or R^{15α} and R^{15β} together are ═O; each of R^{16α} and R^{16β} is, independently, H, OH, OR^{16A}, or OC(O)R^{16A}, where R^{16A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{16α} and R^{16β} together are ═O; R¹⁷ is

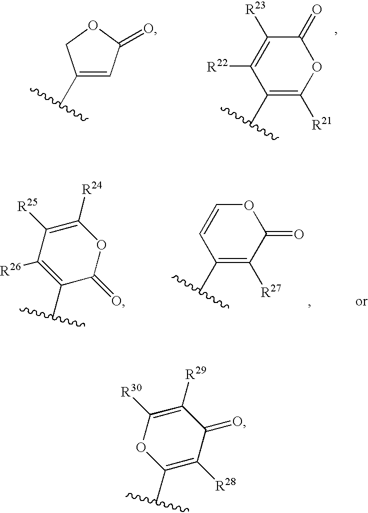

where each of R²¹, R²², R²³, R²⁴, R²⁵, R²⁶, R²⁷, R²⁸, R²⁹, and R³⁰ is, independently, H, C₁₋₇ alkyl, C₂₋₇ alkenyl, C₂₋₇ alkynyl, C₂₋₄ heterocyclyl, C₆₋₂ aryl, C₇₋₁₄ alkaryl, C₃₋₁₀ alkheterocyclyl, or C₁₋₇ heteroalkyl; R^{17α} is H or OH; and R′″ is CH₃, CH₂OR^{18A}, or CH₂OCOR^{18A}, where R^{18A} is H, C₁₋₇ alkyl, C₂₋₇ alkenyl, C₂₋₇ alkynyl, C₂₋₄ heterocyclyl, C₆₋₁₂ aryl, C₇₋₁₄ alkaryl, C₃₋₁₀-alkheterocyclyl, or C₁₋₇ heteroalkyl.

In a further aspect, the invention features a compound of formula IVb:

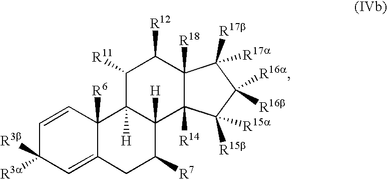

or a pharmaceutically acceptable salt or prodrug thereof. In formula IVb each of R¹, R⁵, R⁷, R¹¹, and R¹² is, independently, H; OH, OR^{1A}, or OC(O)R^{1A}, where R^{1A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; each of R^{3α} and R^{3β} is, independently, H, OR^{3A} or OC(O)R^{3B} and each of R^{3A} and R^{3B} is, independently, C₂₋₆ heterocyclyl, C₆₋₁₂ aryl, C₇₋₁₄ alkaryl, C₃₋₁₀ alkheterocyclyl, or C₁₋₇ heteroalkyl, with the proviso that at least one of R^{3α} and R^{3β} is not H; R⁶ is CH₃, CH₂OR^{6A}, or CH₂OCOR^{6A}, where R^{6A} is H, C₁₋₇ alkyl, C₂₋₇ alkenyl, C₂₋₇ alkynyl, C₂₋₄ heterocyclyl, C₆₋₂ aryl, C₇₋₁₄ alkaryl, C₃₋₁₀ alkheterocyclyl, or C₁₋₇ heteroalkyl; R¹⁴ is OH, Cl, OR^{14A}, or OC(O)R^{14A}, where R^{14A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R¹⁴, R^{15β}, and the carbons they are bonded to together represent an epoxide; each of R^{15α} and R^{15β} is, independently, H, OH, OR^{15A}, or OC(O)R^{15A}, where R^{15A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ allcheterocyclyl, or C₁₋₇ heteroalkyl, or R^{15α} and R^{15β} together are ↑O; each of R^{16α} and R^{16β} is, independently, H, OH, OR^{16A}, or OC(O)R^{16A}, where R^{16A} is C₁₋₇ alkyl, C₂₋₇ alkenyl, C₂₋₇ alkynyl, C₂₋₆ heterocyclyl, C₆₋₂ aryl, C₇₋₁₄ alkaryl, C₃₋₁₀ alkheterocyclyl, or C₁₋₇ heteroalkyl, or R^{16α} and R^{16β} together are ═O; R^{17β} is

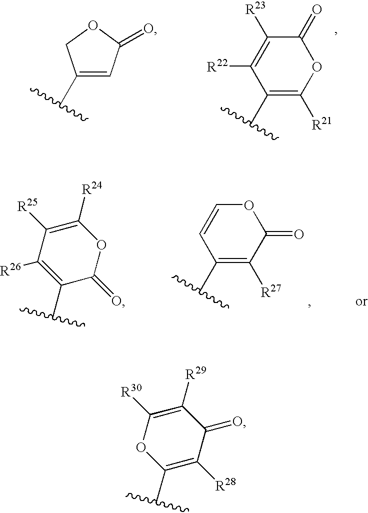

where each of R²¹, W²², R²³, R²⁴, R²⁵, R²⁶, R²⁷, R²⁸, R²⁹, and R³⁰ is, independently, H, C₁₋₇ alkyl, C₂₋₇ alkenyl, C₂₋₇ alkynyl, C₂₋₄ heterocyclyl, C₆₋₁₂ aryl, C₇₋₁₄ alkaryl, C₃₋₁₀ alkheterocyclyl, or C₁₋₇ heteroalkyl; R^{17α} is H or OH; and R¹⁸ is CH₃, CH₂OR^{18A}, or CH₂OCOR^{18A}, where R^{18A} is H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

In an embodiment of compounds having formulas I, II, or III, R^{3α} and R^{3β} together are ═NNR^{3M}R^{3N}, or —NOR^{3P}, wherein each of R^{3M}, R^{3N} and R^{3P} is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl. An exemplary compound of formula I is

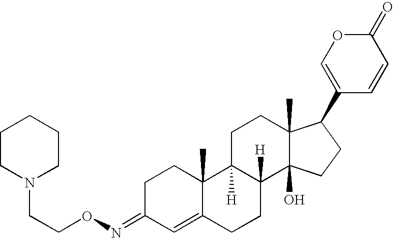

In another aspect, the invention features a method for treating a disorder in a mammal mediated by hypoxia inducible factor-1 (HIF-1) by administering to the mammal a compound of the invention in an amount sufficient to treat the disorder, and the use of the compound in the manufacture of a medicament for such a method. The disorder can be a metabolic disorder, such as syndrome X, obesity, or atherogenic dyslipidemia. The disorder can be a hypertension disorder, such as sleep-disordered breathing, or obstructive sleep apnea. The disorder can be an inflammatory disorder, such as arthritis, psoriasis, or atherosclerosis. The disorder can be characterized by pathogenic angiogenesis. Disorders characterized by pathogenic angiogenesis include, without limitation, ocular disorders, such as optic disc neovascularization, iris neovascularization, retinal neovascularization, choroidal neovascularization, corneal neovascularization, vitreal neovascularization, glaucoma, pannus, pterygium, macular edema, diabetic macular edema, vascular retinopathy, retinal degeneration, uveitis, inflammatory diseases of the retina, excessive angiogenesis following cataract surgery, and proliferative vitreoretinopathy; and neoplastic disorders, such as carcinoma of the bladder, breast, colon, kidney, liver, lung, head and neck, gall-bladder, ovary, pancreas, stomach, cervix, thyroid, prostate, or skin; a hematopoietic cancer of lymphoid lineage, a hematopoietic cancer of myeloid lineage, a cancer of mesenchymal origin, a cancer of the central or peripheral nervous system, melanoma, seminoma, teratocarcinoma, osteosarcoma, thyroid follicular cancer, and Kaposi's sarcoma. The disorder can be Alzheimer's Disease.

In a related aspect, the invention features a method for reducing VEGF expression in a cell by contacting the cell with a compound of the invention in an amount sufficient to reduce VEGF expression.

In yet another aspect, the invention features a method for treating a patient with a neoplastic disorder by administering to the patient (i) a compound of the invention, and (ii) an antiproliferative agent, wherein the compound of the invention and the antiproliferative agent are administered simultaneously, or within 14 days of each other, each in an amount that together is sufficient to treat a neoplastic disorder. The antiproliferative agent can be selected from alkylating agents, folic acid antagonists, pyrimidine antagonists, purine antagonists, antimitotic agents, DNA topoisomerase II inhibitors, DNA topoisomerase I inhibitors, taxanes, DNA intercalators, aromatase inhibitors, 5-alpha-reductase inhibitors, estrogen inhibitors, androgen inhibitors, gonadotropin releasing hormone agonists, retinoic acid derivatives, and hypoxia selective cytotoxins. Desirably, the antiproliferative agent is gemcitabine.

In another aspect, the invention features a kit including: (i) a compound of the invention; and (ii) instructions for administering the compound of the invention to a patient diagnosed with a disorder mediated by hypoxia inducible factor-1 (HIF-1). The kit can further include an antiproliferative agent, formulated separately or together. Desirably, the compound of the invention and antiproliferative agent are formulated together for simultaneous administration.

In a related aspect, the invention features a method for synthesizing a compound of the invention, wherein R^{3α} and R^{3β} together are ═NOR^{3P}. The method includes the step of condensing H₂NOR^{3P} with a 3-oxo cardiolide or 3-oxo bufadienolide, wherein R^{3P} is H, C₁₋₇ alkyl, C₂₋₇ alkenyl, C₂₋₇ alkynyl, C₂₋₆ heterocyclyl, C₆₋₂ aryl, C₇₋₁₄ alkaryl, C₃₋₁₀ alkheterocyclyl, or C₁₋₇ heteroalkyl.

In another aspect, the invention features a method for synthesizing a compound of the invention, wherein R^{3α} or R³β is O-β-amino-Sac from the corresponding azide wherein R^{3α}, or R^{3β} is O-β-azido-Sac. The method includes the step of reducing the corresponding azide to form an amine, wherein β-azido-Sac is described by formula s1 and β-amino-Sac is described by formula s2:

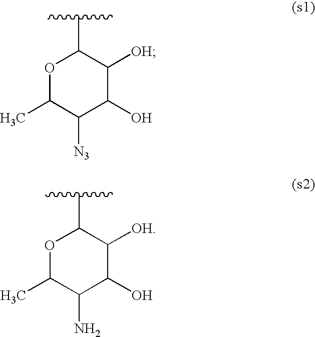

In still another aspect, the invention features a method for synthesizing a compound of the invention, wherein R^{3α} or R^{3β} is O-Sac or NH-Sac. The method includes the step of condensing HO-Sac with a cardiolide or bufadienolide, wherein Sac is described by the formula:

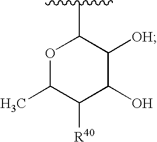

wherein R⁴⁰ is F, Cl, CF₃, OH, NH₂, NHR^{40A}, NR^{40B}R^{40C}, NHC(O)R^{40D}, NHC(S)R^{40E}, NHC(O)OR^{40F}, NHC(S)OR^{40G}, NHC(O)NHR^{40H}, NHC(S)NHR^{40I}, NHC(O)SR^{40J}, NHC(S)SR^{40K}, or NHS(O)₂R^{40L}; and each of R^{40A}, R^{40B}, R^{40C}, R^{40D}, R^{40E}, R^{40F}, R^{40G}, R^{40H}, R^{40I}, R^{40J}, R^{40K}, and R^{40L} is, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{40B} and R^{40C} combine to form a C₂₋₆ heterocyclyl containing at least one nitrogen atom.

In the generic descriptions of compounds of this invention, the number of atoms of a particular type in a substituent group is generally given as a range, e.g. an alkyl group containing from 1 to 7 carbon atoms or C₁₋₇ alkyl. Reference to such a range is intended to include specific references to groups having each of the integer number of atoms within the specified range. For example, an alkyl group from 1 to 7 carbon atoms includes each of C₁, C₂, C₃, C₄, C₅, C₆, and C₇. A C₁₋₇ heteroalkyl, for example, includes from 1 to 6 carbon atoms in addition to one or more heteroatoms. Other numbers of atoms and other types of atoms may be indicated in a similar manner.

As used herein, the terms “alkyl” and the prefix “alk-” are inclusive of both straight chain and branched chain groups and of cyclic groups, i.e. cycloalkyl. Cyclic groups can be monocyclic or polycyclic and preferably have from 3 to 6 ring carbon atoms, inclusive. Exemplary cyclic groups include cyclopropyl, cyclobutyl, cyclopentyl, and cyclohexyl groups. The C₁₋₇ alkyl group may be substituted or unsubstituted. C₁₋₇ alkyls include, without limitation, methyl; ethyl; n-propyl; isopropyl; cyclopropyl; cyclopropylmethyl; cyclopropylethyl; n-butyl; isobutyl; sec-butyl; tert-butyl; cyclobutyl; cyclobutylmethyl; cyclobutylethyl; n-pentyl; cyclopentyl; cyclopentylmethyl; cyclopentylethyl; 1-methylbutyl; 2-methylbutyl; 3-methylbutyl; 2,2-dimethylpropyl; 1-ethylpropyl; 1,1-dimethylpropyl; 1,2-dimethylpropyl; 1-methylpentyl; 2-methylpentyl; 3-methylpentyl; 4-methylpentyl; 1,1-dimethylbutyl; 1,2-dimethylbutyl; 1,3-dimethylbutyl; 2,2-dimethylbutyl; 2,3-dimethylbutyl; 3,3-dimethylbutyl; 1-ethylbutyl; 2-ethylbutyl; 1,1,2-trimethylpropyl; 1,2,2-trimethylpropyl; 1-ethyl-1-methylpropyl; 1-ethyl-2-methylpropyl; and cyclohexyl.

By “C₂₋₇ alkenyl” is meant a branched or unbranched hydrocarbon group containing one or more double bonds and having from 2 to 7 carbon atoms. A C₂₋₇ alkenyl may optionally include monocyclic or polycyclic rings, in which each ring desirably has from three to six members. The C₂₋₇ alkenyl group may be substituted or unsubstituted. C₂₋₇ alkenyls include, without limitation, vinyl; allyl; 2-cyclopropyl-1-ethenyl; 1-propenyl; 1-butenyl; 2-butenyl; 3-butenyl; 2-methyl-1-propenyl; 2-methyl-2-propenyl; 1-pentenyl; 2-pentenyl; 3-pentenyl; 4-pentenyl; 3-methyl-1-butenyl; 3-methyl-2-butenyl; 3-methyl-3-butenyl; 2-methyl-1-butenyl; 2-methyl-2-butenyl; 2-methyl-3-butenyl; 2-ethyl-2-propenyl; 1-methyl-1-butenyl; 1-methyl-2-butenyl; 1-methyl-3-butenyl; 2-methyl-2-pentenyl; 3-methyl-2-pentenyl; 4-methyl-2-pentenyl; 2-methyl-3-pentenyl; 3-methyl-3-pentenyl; 4-methyl-3-pentenyl; 2-methyl-4-pentenyl; 3-methyl-4-pentenyl; 1,2-dimethyl-1-propenyl; 1,2-dimethyl-1-butenyl; 1,3-dimethyl-1-butenyl; 1,2-dimethyl-2-butenyl; 1,1-dimethyl-2-butenyl; 2,3-dimethyl-2-butenyl; 2,3-dimethyl-3-butenyl; 1,3-dimethyl-3-butenyl; 1,1-dimethyl-3-butenyl and 2,2-dimethyl-3-butenyl.

By “C₂₋₇ alkynyl” is meant a branched or unbranched hydrocarbon group containing one or more triple bonds and having from 2 to 7 carbon atoms. A C₂₋₇ alkynyl may optionally include monocyclic, bicyclic, or tricyclic rings, in which each ring desirably has five or six members. The C₂₋₇ alkynyl group may be substituted or unsubstituted. C₂₋₇ alkynyls include, without limitation, ethynyl, 1-propynyl, 2-propynyl, 1-butynyl, 2-butynyl, 3-butynyl, 1-pentynyl, 2-pentynyl, 3-pentynyl, 4-pentynyl, 5-hexene-1-ynyl, 2-hexynyl, 3-hexynyl, 4-hexynyl, 5-hexynyl; 1-methyl-2-propynyl; 1-methyl-2-butynyl; 1-methyl-3-butynyl; 2-methyl-3-butynyl; 1,2-dimethyl-3-butynyl; 2,2-dimethyl-3-butynyl; 1-methyl-2-pentynyl; 2-methyl-3-pentynyl; 1-methyl-4-pentynyl; 2-methyl-4-pentynyl; and 3-methyl-4-pentynyl.

By “C₂₋₄ heterocyclyl” is meant a stable 5- to 7-membered monocyclic or 7- to 14-membered bicyclic heterocyclic ring which is saturated partially unsaturated or unsaturated (aromatic), and which consists of 2 to 6 carbon atoms and 1, 2, 3 or 4 heteroatoms independently selected from the group consisting of N, O, and S and including any bicyclic group in which any of the above-defined heterocyclic rings is fused to a benzene ring. The heterocyclyl group may be substituted or unsubstituted. The nitrogen and sulfur heteroatoms may optionally be oxidized. The heterocyclic ring may be covalently attached via any heteroatom or carbon atom which results in a stable structure, e.g. an imidazolinyl ring may be linked at either of the ring-carbon atom positions or at the nitrogen atom. A nitrogen atom in the heterocycle may optionally be quaternized. Preferably when the total number of S and O atoms in the heterocycle exceeds 1, then these heteroatoms are not adjacent to one another. Heterocycles include, without limitation, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4aH-carbazolyl, β-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylperimidinyl, phenanthridinyl, phenanthrolinyl, phenarsazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred 5 to 10 membered heterocycles include, but are not limited to, pyridinyl, pyrimidinyl, triazinyl, furanyl, thienyl, thiazolyl, pyrrolyl, pyrazolyl, imidazolyl, oxazolyl, isoxazolyl, tetrazolyl, benzofuranyl, benzothiofuranyl, indolyl, benzimidazolyl, 1H-indazolyl, oxazolidinyl, isoxazolidinyl, benzotriazolyl, benzisoxazolyl, oxindolyl, benzoxazolinyl, quinolinyl, and isoquinolinyl. Preferred 5 to 6 membered heterocycles include, without limitation, pyridinyl, pyrimidinyl, triazinyl, furanyl, thienyl, thiazolyl, pyrrolyl, piperazinyl, piperidinyl, pyrazolyl, imidazolyl, oxazolyl, isoxazolyl, and tetrazolyl.

By “C₆₋₁₂ aryl” is meant an aromatic group having a ring system comprised of carbon atoms with conjugated π electrons (e.g. phenyl). The aryl group has from 6 to 12 carbon atoms. Aryl groups may optionally include monocyclic, bicyclic, or tricyclic rings, in which each ring desirably has five or six members. The aryl group may be substituted or unsubstituted.

By “C₇₋₁₄ alkaryl” is meant an alkyl substituted by an aryl group (e.g. benzyl, phenethyl, or 3,4-dichlorophenethyl) having from 7 to 14 carbon atoms.

By “C₃₋₁₀ alkheterocyclyl” is meant an alkyl substituted heterocyclic group having from 7 to 14 carbon atoms in addition to one or more heteroatoms (e.g. 3-furanylmethyl, 2-furanylmethyl, 3-tetrahydrofuranylmethyl, or 2-tetrahydrofuranylmethyl).

By “C₁₋₇ heteroalkyl” is meant a branched or unbranched alkyl, alkenyl, or alkynyl group having from 1 to 7 carbon atoms in addition to 1, 2, 3 or 4 heteroatoms independently selected from the group consisting of N, O, S, and P. Heteroalkyls include, without limitation, tertiary amines, secondary amines, ethers, thioethers, amides, thioamides, carbamates, thiocarbamates, hydrazones, imines, phosphodiesters, phosphoramidates, sulfonamides, and disulfides. A heteroalkyl may optionally include monocyclic, bicyclic, or tricyclic rings, in which each ring desirably has three to six members. The heteroalkyl group may be substituted or unsubstituted.

By “acyl” is meant a chemical moiety with the formula R—C(O)—, wherein R is selected from C₁₋₇ alkyl, C₂₋₇ alkenyl, C₂₋₇ alkynyl, C₂₋₄ heterocyclyl, C₆₋₁₂ aryl, C₇₋₁₄ alkaryl, C₃₋₁₀ alkheterocyclyl, or C₁₋₇ heteroalkyl.

For any of the above definitions, exemplary substituents alkoxy; aryloxy; sulfhydryl; alkylthio; arylthio; halide; hydroxyl; fluoroalkyl; perfluoroalkyl; hydroxyalkyl; alkylsulfinyl; alkylsulfonyl; azido; nitro; OXO; —CO₂R^{A}; —C(O)NR^{B}R^{C}; —SO₂R^{D}; —SO₂NR^{E}R^{F}; and —NR^{G}R^{H}; where each of R^{A}, R^{B}, R^{C}, R^{D}, R^{E}, R^{F}, R^{G}, and R^{H} is, independently, selected from H, C₁₋₇ alkyl, C₂₋₇ alkenyl, C₂₋₇ alkynyl, C₂₋₄ heterocyclyl, C₆₋₁₂ aryl, C₇₋₁₄ alkaryl, C₃₋₁₀ alkheterocyclyl, C₁₋₇ heteroalkyl, and acyl.

By “halide” is meant bromine, chlorine, iodine, or fluorine.

By “fluoroalkyl” is meant an alkyl group that is substituted with a fluorine.

By “perfluoroalkyl” is meant an alkyl group consisting of only carbon and fluorine atoms.

By “hydroxyalkyl” is meant a chemical moiety with the formula —(R)—OH, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “alkoxy” is meant a chemical substituent of the formula —OR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀-alkheterocyclyl, or C₁₋₇ heteroalkyl.

By “aryloxy” is meant a chemical substituent of the formula —OR, wherein R is a C₆₋₁₂ aryl group.

By “alkylthio” is meant a chemical substituent of the formula —SR, wherein R is selected from C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

By “arylthio” is meant a chemical substituent of the formula —SR, wherein R is a C₆₋₁₂ aryl group.

By “saccharide” is meant an aldose or a ketose, either as a monosaccharide or part of a disaccharide or polysaccharide. Saccharides include glycose, glycosamine, aldohexoses, ketohexoses, aldopentose, ketopentose, disaccharides, polysaccharides of 3-20 saccharide units, and deoxy and halide (e.g. fluorinated), amine, alkanoate, sulfate, and/or phosphate derivatives thereof. Suitable monosaccharides include, but are not limited to, any of several simple open or closed chain sugars (in the L or D configuration), typically having 5 or 6 carbons (a pentose monosaccharide or a hexose monosaccharide), as well as 7 carbons (heptose monosaccharide). Included are sugar derivatives in which the ring oxygen atom has been replaced by carbon, nitrogen or sulfur, amino sugars in which a hydroxyl substituent on the simple sugar is replaced with an amino group or sugars having a double bond between two adjacent carbon atoms. Saccharides which can be used in the compounds and methods of the invention include, without limitation, rhamnose, glucose, digitoxose, digitalose, digginose, sarmentose, vallarose, fructose, glucosamine, 5-thio-D-glucose, nojirimycin, deoxynojirimycin, 1,5-anhydro-D-sorbitol, 2,5-anhydro-D-mannitol, 2-deoxy-D-galactose, 2-deoxy-D-glucose, 3-deoxy-D-glucose, allose, arabinose, arabinitol, fucitol, fucose, galactitol, glucitol, iditol, lyxose, mannitol, levo-rhamnitol, 2-deoxy-D-ribose, ribose, ribitol, ribulose, rhamnose, xylose, xylulose, allose, altrose, galactose, gulose, idose, levulose, mannose, psicose, sorbose, tagatose, talose, galactal, glucal, fuical, rhamnal, arabinal, xylal, valienamine, validamine, valiolamine, valiol, valiolon, valienol, valienone, glucuronic acid, galacturonic acid, N-acetylneuraminic acid, gluconic acid D-lactone, galactonic acid γ-lactone, galactonic acid δ-lactone, mannonic acid γ-lactone, D-altro-heptulose, D-manno-heptulose, D-glycero-D-manno-heptose, D-glycero-D-gluco-heptose, D-allo-heptulose, D-altro-3-heptulose, D-glycero-D-mannoheptitol, and D-glycero-D-altro-heptitol, among others). Desirably, the saccharide used in the compounds of the invention is of the formula:

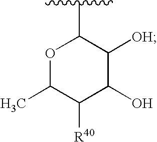

wherein R⁴⁰ is F, Cl, CF₃, OH, NH₂, NHR^{40A}, NR^{40B}R^{40C}, NHC(O)R^{40D}, NHC(S)^{40E}, NHC(O)R^{40F}, NHC(S)OR^{40G}, NHC(O)NHR^{40H}, NHC(S)NHR^{40I}, NHC(O)SR^{40J}, NHC(S)SR^{40K}, or NHS(O)₂R^{40L}, and where each of R^{40A}, R^{40B}, R^{40C}, R^{40D}, R^{40E}, R^{40F}, R^{40G}, R^{40H}, R^{40I}, R^{40J}, R^{40K} and R^{40L} is, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; or R^{40B} and R^{40C} combine to form a C₂₋₆ heterocyclyl containing at least one nitrogen atom.

By “bufadienolide” is meant any compound having a steroid backbone, a hydroxy group or amino group at the C3 position of the steroidal A ring, and a six-membered doubly unsaturated lactone ring substituent at C₁₋₇ of the steroidal D-ring. Examples of bufadienolides are compounds of formulas I, Ia, Ib, II, IIa, IIIb, IV, IVa, or IVb, as described herein, where R^{17β} is:

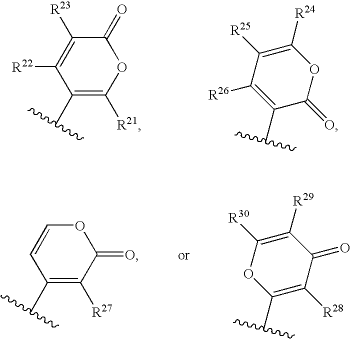

where each of R²¹, R²², R²³, R²⁴, R²⁵, R²⁶, R²⁷, R²⁸, R²⁹, and R³⁰ is as defined elsewhere herein. Thus, in all the above embodiments of compounds having formulas, Ia, Ib, II, IIIa, IIIb, IV, IVa, or IVb, a preferred value for R¹⁷ is as shown in the above four examples.

More preferably, R^{17β} is

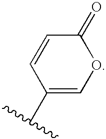

By “3-oxo bufadienolide” is meant any compound having a steroid backbone, an oxo group at the C3 position of the steroidal A ring, and a six-membered doubly unsaturated lactone ring substituent at C17 of the steroidal D-ring.

By “cardiolide” is meant any compound having a steroid backbone, a hydroxy group or amino group at the C3 position of the steroidal A ring, and a five-membered unsaturated lactone ring substituent at C17 of the steroidal D-ring. Examples of cardiolides are those compounds of formulas I, Ia, Ib, II, IIIa, IIIb, IV, IVa, or IVb, as described herein, where R¹⁷ is:

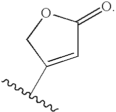

By “3-oxo cardiolide” is meant any compound having a steroid backbone, an oxo group at the C3 position of the steroidal A ring, and a five-membered unsaturated lactone ring substituent at C17 of the steroidal D-ring.

Asymrnetric or chiral centers may exist in any of the compounds of the present invention. The present invention contemplates the various stereoisomers and mixtures thereof. Individual stereoisomers of compounds of the present invention are prepared synthetically from commercially available starting materials which contain asymmetric or chiral centers or by preparation of mixtures of enantiomeric compounds followed by resolution well-known to those of ordinary skill in the art. These methods of resolution are exemplified by (1) attachrnent of a racemic mixture of enantiomers, designated (+/−), to a chiral auxiliary, separation of the resulting diastereomers by recrystallization or chromatography and liberation of the optically pure product from the auxiliary or (2) direct separation of the mixture of optical enantiomers on chiral chromatographic columns. Enantiomers are designated herein by the symbols “R,” or “S,” depending on the configuration of substituents around the chiral carbon atom. Alternatively, enantiomers are designated as (+) or (−) depending on whether a solution of the enantiomer rotates the plane of polarized light clockwise or counterclockwise, respectively.

Geometric isomers may also exist in the compounds of the present invention. The present invention contemplates the various geometric isomers and mixtures thereof resulting from the arrangement of substituents around a carbon-carbon double bond and designates such isomers as of the Z or E configuration, where the term “Z” represents substituents on the same side of the carbon-carbon double bond and the term “E” represents substituents on opposite sides of the carbon-carbon double bond. It is also recognized that for structures in which tautomeric forms are possible, the description of one tautomeric form is equivalent to the description of both, unless otherwise specified.

As used herein, the term “pharmaceutically acceptable salt” refers to those salts which are suitable for use in contact with the tissues of humans and animals without undue toxicity, irritation, or allergic response. Pharmaceutically acceptable salts are well known in the art. For example, S. M Berge et al. describe Pharmaceutically acceptable salts in detail in J. Pharmaceutical Sciences 66:1-19, 1977. The salts can be prepared in situ during the final isolation and purification of any compound described herein or separately by reacting the free base group with a suitable organic acid.

The term “prodrug,” as used herein, represents compounds which are rapidly transformed in vivo to the parent compound of the above formula, for example, by hydrolysis in blood. Prodrugs of the any compound described herein may be conventional esters that are hydrolyzed to their active carboxylic acid form. Some common esters which have been utilized as prodrugs are phenyl esters, aliphatic (C₈-C₂₄) esters, acyloxymethyl esters, carbamates and amino acid esters. In another example, any compound described herein that contains an OH group may be acylated at this position in its prodrug form. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins et al., Synthetic Communications 26(23): 4351-4367, 1996, each of which is incorporated herein by reference.

By an amount “sufficient” is meant the amount of a compound of the invention required to treat a disorder mediated by a local or general hypoxic response. This amount, an amount sufficient, can be routinely determined by one of skill in the art, by animal testing and/or clinical testing, and will vary, depending on several factors, such as the particular disorder to be treated and the particular compound of the invention used. This amount can further depend upon the subject's weight, sex, age and medical history.

As used herein, the term “treatment” refers to the administration of a compound of the invention in an amount sufficient to, alleviate, ameliorate, or delay the progress of one or more symptoms or conditions associated with a disorder mediated by a local or general hypoxic response.

The term “administration” or “administering” refers to a method of giving a dosage of a pharmaceutical composition to a subject, where the method is, e.g., topical, transdermal, oral, intravenous, intraperitoneal, intracerebroventricular, intrathecal, or intramuscular. The preferred; method of administration can vary depending on various factors, e.g. the components of the pharmaceutical composition, site of administration, and severity of the symptoms being treated.

The compounds of the invention can be more efficacious and more easily administered (e.g. orally) in comparison to the prior art compounds BNC1 and BNC4.

Other features and advantages of the invention will be apparent from the following Detailed Description, the drawings, and the claims.

### BRIEF DESCRIPTION OF THE DRAWINGS

FIG. 1 is a schematic diagram showing the adaptation of a cell to hypoxia, which leads to activation of multiple survival factors. The HIF family acts as a master switch transcriptionally activating many genes and enabling factors necessary for glycolytic energy metabolism, angiogenesis, cell survival and proliferation, and erythropoiesis. The level of HIF proteins present in the cell is regulated by the rate of their synthesis in response to factors such as hypoxia, growth factors, androgens and others. Degradation of HIF depends in part on levels of reactive oxygen species (ROS) in the cell. ROS leads to ubiquitylation and degradation of HIF.

FIG. 2 is a Western blot analysis comparison of ouabain (BNC1) and BNC4 in inhibiting hypoxia-mediated HIF-1α induction in human tumor cells (Caki-1 and Panc-1 cells).

FIG. 3 is a Western blot analysis showing that proscillaridin (BNC4) blocks HIF-1α induction by a prolyl-hydroxylase inhibitor (mimosine) under normoxia.

FIGS. 4A-4D are graphs depicting FACS analysis of beta-gal activity in an A549 sentinel line treated with 5 nM of BNC4 (FIG. 4A), BP228 (FIG. 4B), and BP244 (FIG. 4C) in comparison to vehicle only (shown as the shaded portion of the graph) for 24 hours. The graphs indicate frequency of cells (Y-axis) and intensity of fluorescence (X-axis) as measure of pathway activity. The bar chart (FIG. 4D) depicts the relative median fluorescent units of FACS curves.

FIGS. 5A and 5B are a Western blot analysis showing inhibition of hypoxia-mediated HIF-1α induction in Caki-1 (renal cancer, FIG. 5A), A549 (lung cancer, FIG. 5A), Panc-1 (pancreatic cancer; FIG. 5A) and Hep3B (liver cancer, FIG. 5B) cells treated with BNC4, BP228 and BP244 under hypoxic conditions. These results indicate that the compounds are specific and do not inhibit general protein synthesis.

FIG. 6 is two graphs depicting the effect of BP228 and BP244 on secretion of VEGF. Caki-1 cells were treated with indicated compound and cultured under hypoxia for 16 hours. VEGF levels in conditioned medium were measured using an ELISA kit.

FIGS. 7A-7E are graphs depicting the stress response of A549 Sentinel Line induced by treatment with Gemcitabine (FIG. 7A) or Gemcitabine in the presence of indicated compound (FIG. 7B-7D). Untreated (control) sample is shown in shadow. The bar graph (FIG. 7E) shows relative (to control) level of fluorescent intensity. These data show that BNC4, BP228 and BP244 can inhibit the stress response in A549 sentinel line induced by Gemcitabine. Similar results can be achieved for other chemotherapeutic agents which induce hypoxic stress, such as paclitaxel, carboplatin, and mitoxantrone.

FIG. 8 is a graph depicting the mRNA levels of α-1 and α-3 isoforms quantitated by real time RT-PCR (TaqMan) using fluorescent labeled TaqMan probes. Anti-proliferation (IC₅₀ values) activity of BNC4 on indicated cell lines was determined by MTS assay. Total alpha levels (al+a3) were plotted against (1/IC₅₀) X100 values. FIG. 8 shows that there is strong correlation between expression levels of alpha (α1+α3) subunits and anti-proliferation activity of BNC4. Cell lines SNB75 (CNS) and RPMI-8226 (leukemia) expressing very low levels of α-chain are very resistant to BNC4 when compared with A549 (Lung cancer) or PC-3 (prostate cancer) cell lines.

FIG. 9 is a graph depicting the dose dependent effect of BNC4, BP228, and BP244 on the rate of Pi release by Na-K-ATPase. The potency (IC₅₀) to inhibit the activity of Na-K-ATPase from pig brain for each compound is indicated in the brackets.

FIG. 10 is a graph depicting the in vivo activity against renal cancer cell line Caki-1 for BP244.

FIGS. 11A and 11B are graphs depicting the in vivo activity of BP244 in alone (FIG. 11A) and in combination with gemcitabine (FIG. 11B) against pancreatic cancer. As shown in FIG. 11A, BP244 at 15 mg/ml was equivalent to 10 mg/ml with TGI (as used herein, TGI refers to tumor growth inhibition) of almost 100%. At 5 mg/ml, BP244 (TGI 71%) was as effective as Gemcitabine (TGI 65%). Combination therapy using both Gemcitabine and BP244 produces a combination effect (TGI 94%), such that sub-optimal doses of both Gemcitabine (40 mg/kg) and BP244, when used together, produce the maximal effect only achieved by higher doses of individual agents alone.

FIG. 12 is a graph depicting the in vivo activity of BP228 in alone and in combination with gemcitabine against pancreatic cancer. Anti-tumor activity of BP228 against Panc-1 xenografts was determined at 10 mg/ml and 15 mg/ml with and without Gemcitabine (ip; 40 mg/kg, q3d×4). BP228 at 10 mg/ml (TGI 66%) was equivalent in activity to Gemcitabine (TGI 65%), while combinations of BP228 (10 mg/ml) and Gemcitabine (40 mg/kg, q3d×4) gave TGI of 93%.

FIG. 13 is a graph depicting the pharmacokinetic profiled of BNC4, BP228 and BP244 in mice. The compounds were administered by intraperitoneal (i.p) injection at 2.5 mg/kg and 5.0 mg/kg for BP228 and at 5.0 mg/kg for BNC4 and BP244. The plasma samples were collected at various time points and concentration of compounds was analyzed by LC-MS. Pharmacokinetic parameters are provided in Example 23.

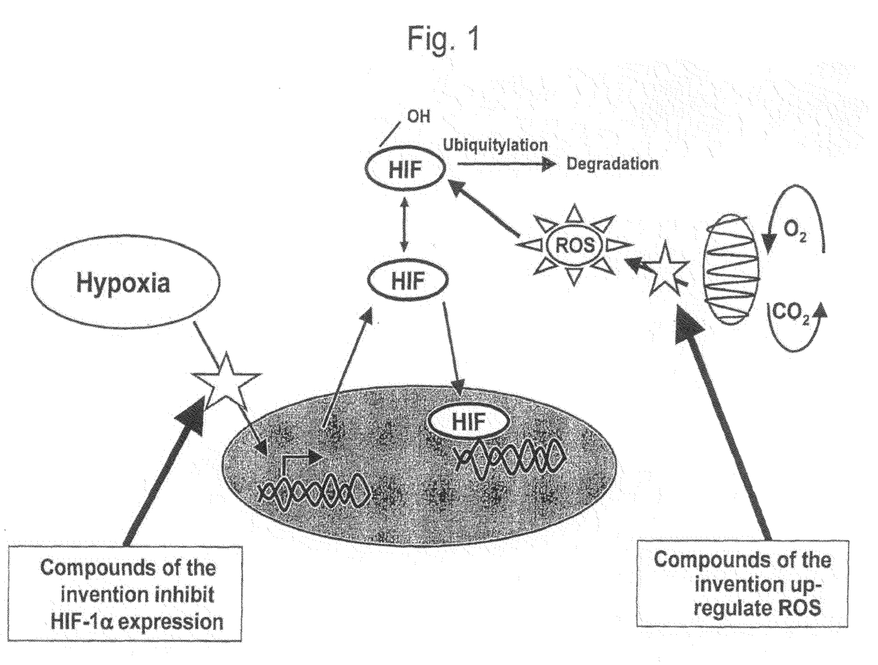

### DETAILED DESCRIPTION

The present invention is based in part on the discovery of compounds which can modulate the effects that are observed as a result of cellular or systemic hypoxia. One salient feature of the present invention is the discovery that certain agents induce an hypoxic stress response and expression of angiogenig factors (such as VEGF) in cells, and that the compounds of the invention can be used to reduce that response. Since hypoxic stress response is associated with the expression of certain angiogenesis factors, including (but not limited to) VEGF, administration of a compound of the invention for inhibiting hypoxic stress response would also inhibit VEGF (and other angiogenesis factors) mediated angiogenesis.

### Metabolic Disorders

The compounds of the invention can be useful for the treatment of metabolic disorders such as, for example, hyperglycemia, impaired glucose tolerance, metabolic syndrome (e.g. Syndrome X), glucosuria, metabolic acidosis, cataracts, diabetic neuropathy and nephropathy, obesity, hyperlipidemia, and metabolic acidosis.

Metabolic syndrome X is a constellation of metabolic disorders that all result from the primary disorder of insulin resistance. All the metabolic abnormalities associated with syndrome X can lead to cardiovascular disorders. When present as a group, the risk for cardiovascular disease and premature death are very high. The characteristic disorders present in metabolic syndrome X include: insulin resistance, hypertension, abnormalities of blood clotting, low HDL and high LDL cholesterol levels, and high triglyceride levels. For the treatment of Syndrome X, the compounds of the invention can be used alone, or in combination with any existing anti-diabetic agent. Agents which may be used in combination with the compounds of the invention include, without limitation, insulin, insulin analogs (e.g. mecasermin), insulin secretagogues (e.g. nateglinide), biguanides (e.g. metformin), sulfonylureas (e.g. chlorpropamide, glipizide, or glyburide), insulin sensitizing agents (e.g. PPARγ agonists, such as troglitazone, pioglitazone, or rosiglitazone), α-glucosidase inhibitors (e.g. acarbose, voglibose, or miglitol), aldose reductase inhibitors (e.g. zopolrestat), metiglinides (e.g. repaglinide), glycogen phosphorylase inhibitors, and GLP-1 and functional mimetics thereof (e.g. exendin-4), among others.

Obesity may result from or be associated with a variety of phenotypes, many of which are reflective of a hypoxic condition. For example, many individuals suffering from chronic hypoxia crave carbohydrates, and carbohydrate cravings are also common in obese individuals. It is thought that adipose tissue exhibits angiogenic activity and also that adipose tissue mass can be regulated via the vasculature. There is reciprocal paracrine regulation of adipogenesis and angiogenesis. Furthermore, it has been shown that a blockade of vascular endothelial growth factor (VEGF) signaling can inhibit in vivo adipose tissue formation. Fukumura et al. in Circulation Research 93:e88-97, 2003.

The present invention features methods for down-regulating angiogenetic factors to inhibit angiogenesis in vivo in treating/preventing obesity, by administering a compound of the invention, with or without other anti-angiogenesis factors.

For the treatment of obesity, a compound of the invention may be used alone, or in combination with any existing anti-obesity agent, such as those described by Flint et al., J. Clin. Invest. 101:515-520, 1998 or by Toft-Nielsen et al., Diabetes Care 22:1137-1143, 1999. Agents which may be used in combination with the compounds of the present invention include, without limitation, fatty acid uptake inhibitors (e.g. orlistat), monoamine reuptake inhibitors (e.g. sibutramine), anorectic agents (e.g. dexfenfluramine or bromocryptine), sympathomimetics (e.g. phentermine, phendimetrazine, or mazindol), and thyromimetic agents, among others.

### Hypertensive Disorders

The compounds and methods of the invention can be useful for the treatment of hypertension. Systemic hypertension is the most prevalent cardiovascular disorder in the United States, affecting more than 50 million individuals. Hypertension is a common cause of major medical illnesses, including stroke, heart disease, and renal failure, in middle-aged males. Its prevalence in the United States is around 20%, with the rate of newly diagnosed hypertensive patients being about 3% per year.

Obstructive sleep apnea syndrome is common in the same population. It is estimated that up to 2% of women and 4% of men in the working population meet criteria for sleep apnea syndrome. The prevalence may be much higher in older, non-working men. Many of the factors predisposing to hypertension in middle age, such as obesity, are also associated with sleep apnea. Recent publications describe a 30% prevalence of occult sleep apnea among middle-aged males with hypertension. In addition, an association has also been found for hypertension and sleep-disordered-breathing (see, for example, Fletcher, Am. J. Med. 98(2): 118-28, 1995).

HIF-1, as one of the pivotal mediators in the response to hypoxia, has been implicated in the pathogenesis of hypertension (see, for example, Li and Dai, Chin. Med. J. (Engl). 117(7): 1023-8, 2004; and Semenza, Genes and Development 14:1983-1991, 2000). Due to their ability to decrease HIF-expression, a compound of the invention can be useful for the treatment of disorders caused by hypertension, such as sleep-disordered breathing and obstructive sleep apnea.

### Angiogenic Disorders

The compounds of the invention are potent inhibitors of HIF-1, which is itself a potent activator of pro-angiogenic factors. While not wishing to be bound to any particular mechanism, it is reasonable to expect that a factor involved in mounting a global response to hypoxia would suppress local responses, such as angiogenesis, that would be inappropriate if local cellular hypoxia is attributable to systemic disturbances in ventilation or oxygen supply.

The compositions and methods of the invention can be used to inhibit angiogenesis which is nonpathogenic, i.e. angiogenesis which results from normal biological processes in the subject. Besides during embryogenesis, angiogenesis is also activated in the female reproductive system during the development of follicles, corpus luteum formation and embryo implantation. During these processes, angiogenesis is mediated mainly by VEQF. Uncontrolled angiogenesis may underlie various female reproductive disorders, such as prolonged menstrual bleeding or infertility, and excessive endothelial cell proliferation has been observed in the endometrium of women with endometriosis. Neovascularization also plays a critical role in successful wound healing that is probably regulated by IL-8 and the growth factors FGF-2 and VEGF. Macrophages, known cellular components of the accompanying inflammatory response, may contribute to the healing process by releasing these angiogenic factors. Examples of non-pathogenic angiogenesis include endometrial neovascularization, and processes involved in the production of fatty tissues or cholesterol. Thus, the invention provides a method for inhibiting non-pathogenic angiogenesis, e.g. for controlling weight or promoting fat loss, for reducing cholesterol levels, or as an abortifacient.

The compositions and methods of the invention can also be used to inhibit angiogenesis which is pathogenic, i.e. a disease in which pathogenicity is associated with inappropriate or uncontrolled angiogenesis. For example, most cancerous solid tumors generate an adequate blood supply for themselves by inducing angiogenesis in and around the tumor site. This tumor-induced angiogenesis is often required for tumor growth, and also allows metastatic cells to enter the bloodstream. Furthermore, numerous ocular diseases are associated with uncontrolled or excessive angiogenesis.

Neoplastic disorders associated with angiogenesis that can be treated using the compounds and methods of the invention include, without limitation, tumor growth, hemangioma, meningioma, solid tumors, leukemia, neovascular glaucoma, angiofibroma, pyogenic granuloma, scleroderma, trachoma, and metastasis thereof.

Non-neoplastic disorders associated with angiogenesis that can be treated using the compounds and methods of the invention include, without limitation, retinal neovascularization, diabetic retinopathy, retinopathy of prematurity (ROP), endometriosis, macular degeneration, age-related macular degeneration (ARMD), psoriasis, arthritis, rheumatoid arthritis (RA), atherosclerosis, hemangioma, Kaposi's sarcoma, thyroid hyperplasia, Grave's disease, arterioyenous malformations (AVM), vascular restenosis, dermatitis, hemophilic joints, hypertrophic scars, synovitis, vascular adhesions, and other inflammatory diseases.

The compounds and methods of the invention can also be useful for preventing or alleviating abnormal angiogenesis following cataract surgery. In normal lenses, immunoreactivity against bufalin and ouabain-like factor is sevenfold to 30-fold higher in the capsular epithelial layer than in the lens fiber region (Lichtstein et al., Involvement of Na+, K+-ATPase inhibitors in cataract formation, in Na/K-ATPase and Related ATPases, 2000, Taniguchi, K. & Haya, S., eds, Elsevier Science, Amsterdam). In human cataractous lenses, the concentration of the sodium pump inhibitor was much higher than in normal lenses. Hence, it was isolated from cataractous lenses and identified as 19-norbufalin and its Thr-Gly-Ala tripeptide derivative (Lichtstein et al., Eur. J. Biochem. 216:261-268, 1993). Cataract surgery will remove such steroids, resulting in the possible loss of the local inhibition of unwanted angiogenesis in the eye. Patients after cataract surgery may therefore be more vulnerable to conditions associated with abnormal angiogenesis.

### Inflammatory Disorders

Angiogenesis and enhanced microvascular permeability are hallmarks of a large number of inflammatory diseases. Angiogenesis and chronic inflammation are closely linked (Jackson et al., FASEB J. 11:457-465, 1997). Angiogenic blood vessels at the site of inflammation are enlarged and hyperpermeable to maintain the blood flow and to meet the increased metabolic demands of the tissue (Jackson et al., Supra). Several proangiogenic factors, including vascular endothelial growth factor (VEGF) (Detmar, J. Dermatol. Sci. 24 (suppl 1):S78-S84, 2000; Brown et al., J. Invest. Dermatol. 104:744-749, 1995; Fava et al, J. Exp. Med. 180: 341-346, 1994) and members of the CXC-chemokine family (Schroder and Mochizuki, Biol. Chem. 380: 889-896, 1999; Strieter et al., Shock 4: 155-160, 1995) have been found to be up-regulated during inflammation. While not wishing to be bound by any particular theory, inflammation may induce local hypoxia response and promote angiogenesis through, for example, VEGF and other factors. Furthermore, immune cells tend to have a constitutively high level of HIF-1. This is coupled with a tendency of these cells to rely on glycolysis. Thus, a number of phenolmena more typically associated with hypoxic cells are constitutively present in certain immune cells.

Accordingly, the compounds and methods of the invention can be used for the treatment of inflammatory diseases, such as rheumatoid arthritis, psoriasis, and atherosclerosis.

### Alzheimer's Disease (AD)

The compounds and methods of the invention can be useful for inhibiting the onset and/or development of AD. Alzheimer's disease (AD), characterized by impairments in cognition and memory, is clearly associated with the slow accumulation of amyloid β peptides (AβPs) in the central nervous system (Selkoe, Physiol. Rev. 81:741-766, 2001; Small et al., Nat. Rev. Neurosci. 2:595-598, 2001). AβPs are generated via amyloidogenic processing of amyloid precursor protein (APP) by β- and γ-secretases, and recent evidence suggests that γ-secretase activity requires the formation of a complex between presenilin, nicastrin, APH-1 and pen-2 (Edbauer et al., Nat. Cell Biol. 5:486-488, 2003). Disruption of Ca²⁺ homeostasis has been strongly implicated in the neurodegeneration of AD; indeed, increased Ca²⁺-dependent protease activity occurs in association with degenerating neurones in AD brain tissue (Nixon et al., Ann. NY Acad. Sci. 747:77-91, 1994), and AβPs perturb Ca²⁺ homeostasis, rendering cells susceptible to excitotoxic damage (Mattson et al., J. Neurosci. 12:376-389, 1992). Presenilin mutations are known to have effects on cellular Ca²⁺ homeostasis (Mattson et al, Trends Neurosci. 23, 222-229, 2000), and familial AD (FAD)-related mutations of presenilin-1 (PS-1) can alter inositol triphosphate-coupled intracellular Ca²⁺ stores as well as Ca²⁺ influx pathways (Leissring et al., J. Cell Biol. 149:793-798, 2000; Mattson et al., Trends Neurosci. 23:222-229, 2000; Yoo et al., Neuron 27:561-572, 2000). This may contribute to neurodegeneration, since disruption of Ca²⁺ homeostasis is an important mechanism underlying such loss of neurones (Chan et al., J. Biol. Chem. 275:18195-18200, 2000; Mattson et al., J. Neurosci. 20:1358-1364, 2000; Yoo et al., supra).

Periods of cerebral hypoxia or ischemia can increase the incidence of AD (Tatemichi et al., Neurology 44:1885-1891, 1994; Kokmen et al., Neurology 46:154-159, 1996), and APP expression is elevated following mild and severe brain ischemia (Kogure and Kato, Stroke 24:2121-2127, 1993). Since the non-amyloidogenic cleavage product of APP (sAPPα) is neuroprotective (Mattson, Physiol. Rev. 77:1081-1132, 1997; Selkoe, Physiol. Rev. 81:741-766, 2001), increased expression during hypoxia could be considered a protective mechanism against ischemia. However, increased APP levels would also provide an increased substrate for AβP formation. It was previously shown that AβP formation is increased following hypoxia in PC12 cells (Taylor et al., J. Biol. Chem. 274:31217-31222, 1999; Green et al., J. Physiol. 541:1013-1023, 2002). Furthermore, prolonged hypoxia potentiates bradykinin (BK)-induced Ca²⁺ release from intracellular stores in rat type I cortical astrocytes. This was due to dysfunction of mitochondria and plasmalemmal Na⁺/Ca²⁺ exchanger (NCX; Smith et al., J. Biol. Chem. 278:4875-4881, 2003). Peers et al., Biol. Chem. 385(34):285-9, 2004 report that sustained central hypoxia predisposes individuals to dementias such as Alzheimer's disease, in which cells are destroyed in part by disruption of Ca²⁺ homeostasis. Moreover, hypoxia increases the levels of presenilin-1, a major component of a key enzyme involved in Alzheimer's disease. Thus there is established link between periods of hypoxia and the development of AD.

### Proliferative Disorders

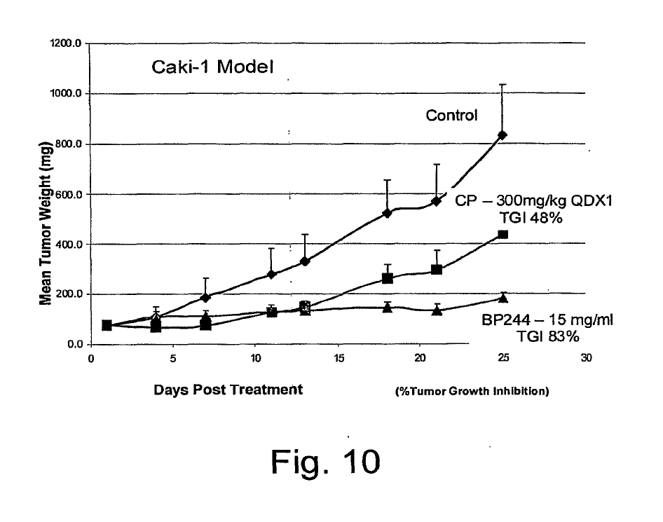

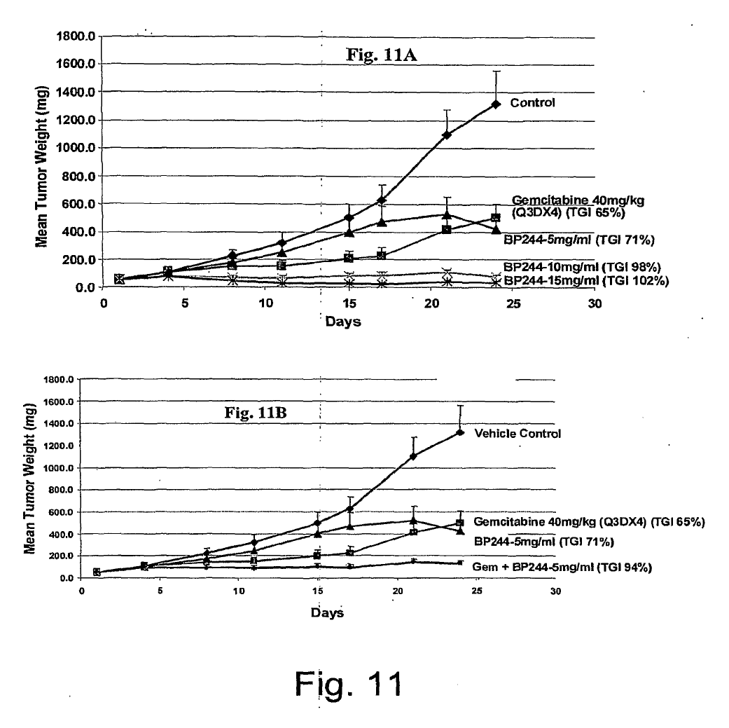

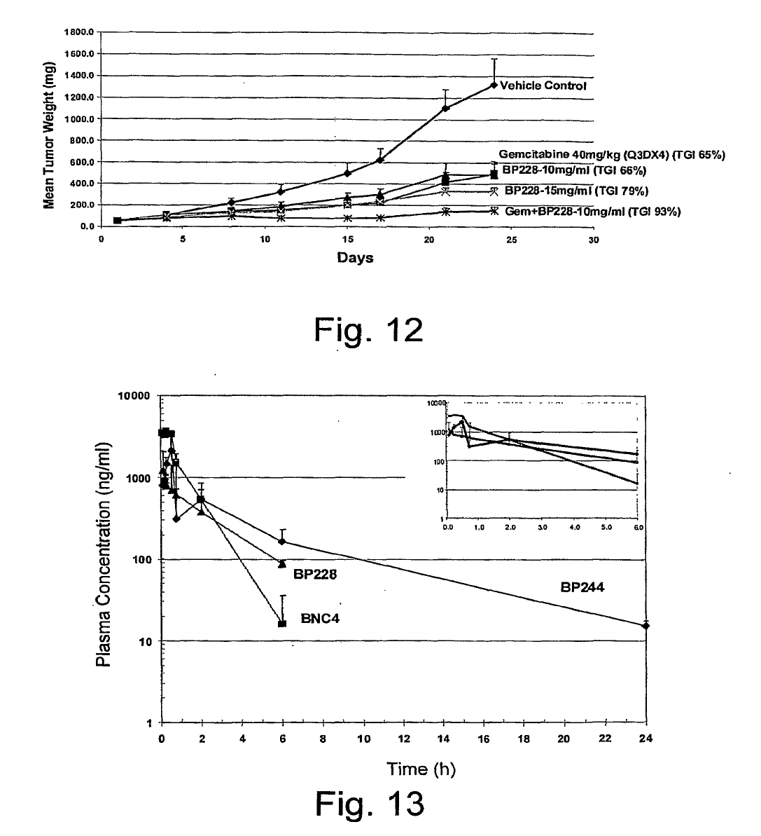

The compounds and methods of the invention can be useful for the treatment of proliferative disorders. Notably, the compounds of the invention can inhibit the proliferation of cancer cell lines at a concentration well below the known toxicity level (see FIGS. 10-13).

### Combination Therapy

The compounds of the invention can be used in combination with other antiproliferative agents for the treatment of cancer and/or inhibiting the formation of metastases. Antiproliferative agents to be used in the combination include, without limitation, those agents provided in Table 1.

Desirably, the compound of the invention is added to an existing clinical regimen (e.g. paclitaxel for the treatment of breast cancer) for the purpose of reducing the minimum efficacious dose. The benefit to the patient is an increase in the therapeutic index of the anticancer agent when used in combination with a compound of the invention. Accordingly, the compound of the invention can be added to any existing cancer therapy regimen for the purpose of reducing adverse drug reactions, extending the life of the patient, and/or improving the cure rate.

TABLE 1
Antiproliferative Agents

| Class | Type of Agent | Nonproprietary Names | Cancers |
|---|---|---|---|
| Alkylating | Nitrogen mustards | Mechlorethamine | Hodgkin's disease, non-Hodgkin's |
| agents | | | lymphomas |
| | | Cyclophosphamide, | Acute and chronic lymphocytic, leu- |
| | | Ifosfamide | kemias, Hodgkin's disease, non-Hodg- |
| | | | kin's lymphomas, multiple myeloma, |
| | | | neuroblastoma, breast, ovary, lung, |
| | | | Wilms' tumor, cervix, testis, soft-tissue |
| | | | sarcomas |
| | | Melphalan | Multiple myeloma, breast, ovary |
| | | Chlorambucil | Chronic lymphocytic leukemia, Primary |
| | | | macroglobulinemia, Hodgkin's disease, |
| | | | non-Hodgkin's lymphomas |
| | | Uracil mustard | Leukemia |
| | | Estramustine | Solid Tumors |
| | Ethylenimines and | Mitomycin C | Colorectal, ocular |
| | Methylmelamines | AZQ | Primary brain tumors |
| | | Thiotepa | Bladder, breast, ovary |
| | Alkyl Sulfonates | Busulfan, Hepsulfam | Chronic myelogenous leukemia |
| | Nitrosoureas | Carmustine | Hodgkin's disease, non-Hodgkin's |
| | | | lymphomas, primary brain tumors, mul- |
| | | | tiple myeloma, malignant melanoma |
| | | Lomustine | Hodgkin's disease, non-Hodgkin's |
| | | | lymphomas, primary brain tumors, small- |
| | | | cell lung |
| | | Semustine | Primary brain tumors, stomach, colon |
| | | Streptozocin | Malignant pancreatic insulinama, |
| | | | malignant carcinoid |
| | Triazines | Dacarbazine | Malignant melanoma, Hodgkin's disease, |
| | | | soft-tissue sarcomas |
| | Platinum | Cisplatin, Carboplatin | Testis, ovary, bladder, head and neck, |
| | Complexes | | lung, thyroid, cervix, endometrium, |
| | | | neuroblastoma, osteogenic sarcoma |
| | Methyl Hydrazine | Procarbazine | Hodgkin's disease |
| | Derivative | | |
| Antime- | Folic Acid Antag- | Methotrexate, Trimetrexate | Acute lymphocytic leukemia, chorio- |
| tabolites | onists | | carcinoma, mycosis fungoides, breast, |
| | | | head and neck, lung, osteogenic sarcoma |
| | Pyrimidine Antag- | Fluouracil, Floxuridine | Breast, colon, stomach, pancreas, ovary, |
| | onists | | head and neck, urinary bladder, skin, |
| | | | adenocarcinomas |
| | | Cytarabine | Acute myelogenous and acute |
| | | | lymphocytic leukemias |
| | | Fludarabine Phosphate | Lymphoproliferative disease |
| | | Capecitabine | Breast, renal cell, prostate |
| | | Azacitidine | acute leukemias |
| | Purine Antagonists | Thioguanine | Acute myelogenous, acute lymphocytic |
| | | | and chronic myelogenous leukemias |
| | | Mercaptopurine | Acute lymphocytic, acute myelogenous |
| | | | and chronic myelogenous leukemias |
| | | Allopurine | leukemias |
| | | Cladribine | Hairy cell leukemia |
| | | Gemcitabine | Pancreatic, soft tissue carcinomas |
| | | Pentostatin | Hairy cell leukemia, mycosis fungoides; |
| | | | chronic lymphocytic leukemia |
| Antimitotic Agents | | Vinblastine | Hodgkin's disease, non-Hodgkin's |
| | | | lymphomas, breast, testis |
| | | Vincristine | Acute lymphocytic leukemia, neuro- |
| | | | blastoma, Wilms' tumor, rhabdo- |
| | | | myosarcoma, Hodgkin's disease, non- |
| | | | Hodgkin's lymphomas, small-cell lung |
| DNA Topoisomerase II Inhibitors | | Etoposide, Teniposide | Testis, small-cell lung, oat-cell lung, |
| | | | breast, Hodgkin's disease, non-Hodgkin's |
| | | | lymphomas, acute myelogenous leu- |
| | | | kemia, Kaposi's sarcoma |
| DNA Topoisomerase I Inhibitors | | Topotecan, Irinotecan, | Ovarian, colorectal |
| | | Camptothecin, 9-Amino- | |
| | | camptothecin | |
| Taxanes | | Paclitaxel, Docetaxel | Breast |
| DNA Intercalators | | Daunorubicin | Acute myelogenous and acute |
| | | | lymphocytic leukemias |
| | | Doxorubicin | Ewing's sarcoma, osteosarcoma, rhabdo- |
| | | | myosarcomas, Hodgkin's disease, non- |
| | | | Hodgkin's lymphomas, acute leukemias, |
| | | | multiple myeloma, breast, genitourinary, |
| | | | thyroid, lung, ovarian, endometrial, |
| | | | testicular, stomach, neuroblastoma |
| | | Dactinomycin | Choriocarcinoma, Wilms' tumor, rhabdo- |
| | | | myosarcoma, testis, Kaposi's sarcoma |
| | | Idarubincin | Acute myeloid leukemia |
| | | Plicamycin | Testicular cancer |
| | | Mitomycin | Squamous sell carcinomas, small bladder |
| | | | papillomas, adenocarcinomas, pancreas, |
| | | | lung, colon, stomach, cervix, breast, head |
| | | | and neck |
| | | Amsacrine | Acute myelogenous leukemia, ovarian |
| | | | cancer, lymphomas |
| | | Bleomycin | Testicular, head and neck, skin, |
| | | | esophagus, squamous cell, colorectal, |
| | | | lung, genitourinary tract, cervix, ovarian, |
| | | | breast, Hodgkin's disease, non-Hodgkin's |
| | | | lymphomas |
| Hormonal | Aromatase | Aminoglutethimide, | Breast |
| Agents | Inhibitors | Anastrozole | |
| | 5-alpha-Reductase | Finasteride, Ketoconazole | Prostate |
| | Inhibitors | | |
| | Estrogen and | Tamoxifen | Breast |
| | Androgen | Flutamide | Prostate |
| | Inhibitors | | |
| | Gonadotropin | Leuprolide, Goserelin | Prostate |
| | Releasing | | |
| | Hormone Agonists | | |
| Tyrosine | ABL Inhibitors | Gleevec ™ (Novartis) | chronic myelogenous leukemia or acute |
| Kinase In- | | | lymphoblastic leukemia |
| hibitors | PDGFR Inhibitors | Leflunomide (Pharmacia), | gastrointestinal stromal tumor, small cell |
| | | SU5416 (Pharmacia), SU6668 | lung cancer, glioblastoma multiforme, |
| | | (Pharmacia), PTK787 | and prostate cancer |
| | | (Novartis) | |
| | EGFR Inhibitors | Iressa ™ (AstraZeneca), | non-small-cell lung cancer, breast cancer, |
| | | Tarceva ™, (Oncogene | ovarian cancer, bladder cancer, prostate |
| | | Science), trastuzumab | cancer, salivary gland cancer, pancreatic |
| | | (Genentech), Erbitux ™ | cancer, endometrial cancer, colorectal |
| | | (ImClone), PK1166 (Novartis), | cancer, kidney cancer, head and neck |
| | | GW2016(Glaxo- | cancer, glioblastoma multiforme |
| | | SmithKline), EKB-509 | |
| | | (Wyeth), EKB-569 (Wyeth), | |
| | | MDX-H210 (Medarex), 2C4 | |
| | | (Genentech), MDX-447 | |
| | | (Medarex), ABX-EGF | |
| | | (Abgenix), CI-1033 (Pfizer) | |
| | VEGFR Inhibitors | Avastin ™ (Genentech), IMC- | any solid tumor |
| | | 1C11 (ImClone), ZD4190 | |
| | | (AstraZeneca), ZD6474 | |
| | | (AstraZeneca ) | |
| | Trk Inhibitors | CEP-701 (Cephalon), CEP- | prostate cancer, pancreatic cancer |
| | | 751 (Cephalon) | |
| | Flt-3 Inhibitors | MLN518 (Millennium), | acute myeloid leukemia |
| | | PKC412 (Novartis) | |

| | | |
|---|---|---|
| Retinoic Acid Derivatives | 13-cis-retinoic acid, iso- | Acute promyelocytic leukemia, head and |
| | tretinoin, retinyl palmitate, 4- | neck squamous cell carcinoma |
| | (hydroxycarbophenyl) | |
| | retinamide | |
| Hypoxia-Selective Cytoxins | Misonidazole | Head and neck |
| | Nitracrine | Breast |
| Miscellaneous Agents | Mitoxantrone | Acute myelogenous leukemia non- |
| | | Hodgkin's lymphoma's, breast |
| | Hydroxyurea | Chronic myelogenous leukemia, |
| | | polycythemia vera, essential thrombo- |
| | | cytosis, malignant melanoma |
| | L-Asparaginase | Acute lymphocytic leukemia |
| | Interferon alfa | Hairy cell leukemia., Kaposi's sarcoma, |
| | | melanoma, carcinoid, renal cell, ovary, |
| | | bladder, non-Hodgkin's lymphomas, |
| | | mycosis fungoides, multiple myeloma, |
| | | chronic myelogenous leukemia |
| | Rapamycin, CCI-779 | Glioblastoma Multiforme, renal cell |
| | | carcinoma |
| | Mitotane | Adrenal carcinoma |

In the methods of the present invention, the dosage and frequency of administration of the compound of the invention and additional anti-proliferative agent(s) can be controlled independently. For example, one compound may be administered orally three times per day, while the second compound may be administered intravenously once per day. The compounds may also be formulated together such that one administration delivers both compounds.

The exemplary dosage of the compound of the invention and additional antiproliferative agent(s) to be administered will depend on such variables as the type and extent of the disorder, the overall health status of the patient, the therapeutic index of the selected antiproliferative agent(s), and their route of administration. Standard clinical trials may be used to optimize the dose and dosing frequency for any particular combination of the invention.

### Administration

The invention features compositions and methods that can be used to modulate the effects of local and systemic hypoxic events. The compounds of the invention can be formulated with a pharmaceutically acceptable excipient prior to administration. These pharmaceutical compositions can be prepared according to the customary methods, using one or more pharmaceutically acceptable adjuvants or excipients. The adjuvants comprise, without limitation, diluents, sterile aqueous media, and various non-toxic organic solvents. Acceptable carriers or diluents for therapeutic use are well known in the pharmaceutical field, and are described, for example, in Remington: The Science and Practice of Pharmacy (20th ed.), ed. A. R. Gennaro, Lippincott Williams & Wilkins, 2000, Philadelphia, and Encyclopedia of Pharmaceutical Technology, eds. J. Swarbrick and J. C. Boylan, 1988-1999, Marcel Dekker, New York. The compositions may be presented in the form of tablets, pills, granules, powders, aqueous solutions or suspensions, injectable solutions, elixirs, or syrups, and the compositions may optionally contain one or more agents chosen from the group comprising sweeteners, flavorings, colorings, and stabilizers in order to obtain pharmaceutically acceptable preparations.

Dosage levels of active ingredients in the pharmaceutical compositions of the invention may be varied to obtain an amount of the active compound(s) that achieves the desired therapeutic response for a particular patient, composition, and mode of administration. The selected dosage level depends upon the activity of the particular compound, the route of administration, the severity of the condition being treated, and the condition and prior medical history of the patient being treated. For adults, the doses are generally from about 0.01 to about 100 mg/kg, desirably about 0.1 to about 1 mg/kg body weight per day by inhalation, from about 0.01 to about 100 mg/kg, desirably 0.1 to 70 mg/kg, more desirably 0.5 to 10 mg/kg body weight per day by oral administration, and from about 0.01 to about 50 mg/kg, desirably 0.1 to 1 mg/kg body weight per day by intravenous administration. Doses are determined for each particular case using standard methods in accordance with factors unique to the patient, including age, weight, general state of health, and other factors which can influence the efficacy of the compound(s) of the invention.

The compound of the invention can be administered orally, parenterally by intravenous injection, transdermally, by pulmonary inhalation, by intravaginal or intrarectal insertion, by subcutaneous implantation, intramuscular injection or by injection directly into an affected tissue, as for example by injection into a tumor site. In some instances the materials may be applied topically at the time surgery is carried out. In another instance the topical administration may be ophthalmic, with direct application of the therapeutic composition to the eye.

For example, the compound of the invention can be administered to a patient by using an osmotic pump, such as the Alzet® Model 2002 osmotic pump. Osmotic pumps provides continuous delivery of test agents, thereby eliminating the need for frequent, round-the-clock injections. With sizes small enough even for use in mice or young rats, these implantable pumps have proven invaluable in predictably sustaining compounds at therapeutic levels, avoiding potentially toxic or misleading side effects.

Alternatively, the compound of the invention can be administered to a patient's eye in a controlled manner. There are numerous devices and methods for delivering drugs to the eye. For example, U.S. Pat. No. 6,331,313 describes various controlled-release devices which are biocompatible and can be implanted into the eye. The devices described therein have a core comprising a drug and a polymeric outer layer which is substantially impermeable to the entrance of an environmental fluid and substantially impermeable to the release of the drug during a delivery period, and drug release is effected through an orifice in the outer layer. These devices have an orifice area of less than 10% of the total surface area of the device and can be used to deliver a variety of drugs with varying degrees of solubility and or molecular weight. Methods are also provided for using these drug delivery devices. The biocompatible, implantable ocular controlled-release drug delivery device is sized for implantation within an eye for continuously delivering a drug within the eye for a period of at least several weeks. Such device comprises a polymeric outer layer that is substantially impermeable to the drug and ocular fluids, and covers a core comprising a drug that dissolves in ocular fluids, wherein the outer layer has one or more orifices through which ocular fluids may pass to contact the core and dissolve drug, and the dissolved drug may pass to the exterior of the device. The orifices in total may have an area less than one percent of the total surface area of the device, and the rate of release of the drug is determined solely by the composition of the core and the total surface area of the one or more orifices relative to the total surface area of the device. Other examples ocular implant methods and devices, and related improvements for drug delivery in the eye are described in U.S. Pat. Nos. 5,824,072, 5,766,242, 5,632,984, 5,443,505, and 5,902,598; U.S. Patent Application US20040175410A1, US20040151754A1, US20040022853A1, US20030203030A1; and PCT publications WO9513765A1, WO0130323A2, WO0202076A2, WO0243785A2, and WO2004026106A2.

For certain applications the compound of the invention may be need to be delivered locally. In such cases, various known methods in the art may be used to achieve limited local delivery without causing undesirable systemic side effects. To just name a few, WO03066130A2 (entire contents incorporated herein by reference) discloses a transdermal delivery system including a drug formulated with a transport chaperone moiety that reversibly associates with the drug. The chaperone moiety is associated with the drug in the formulation so as to enhance transport of the drug across dermal tissue and releasing the drug after crossing said dermal tissue. The application also provides a micro-emulsion system for transdermal delivery of a steroidal HIF-1 modulator, which system solubilizes both hydrophilic and hydrophobic components. For instance, the microemulsion can be a cosolvent system including a lipophilic solvent and an organic solvent. Exemplary cosolvents are NMP and IPM.

International Patent Application WO02087586A1 discloses a sustained release system that includes a polymer and a prodrug having a solubility less than about 1 mg/ml dispersed in the polymer. Advantageously, the polymer is permeable to the prodrug and may be non-release rate limiting with respect to the rate of release of the prodrug from the polymer. This permits improved drug delivery within a body in the vicinity of a surgery via sustained release rate kinetics over a prolonged period of time, while not requiring complicated-manufacturing processes.

The materials are formulated to suit the desired route of administration. The formulation may comprise suitable excipients include pharmaceutically acceptable buffers, stabilizers, local anesthetics, and the like that are well known in the art. For parenteral administration, an exemplary formulation may be a sterile solution or suspension; for oral dosage, a syrup, tablet or palatable solution; for topical application, a lotion, cream, spray or ointment; for administration by inhalation, a microcrystalline powder or a solution suitable for nebulization; for intravaginal or intrarectal administration, pessaries, suppositories, creams or foams.

### Compounds

Compounds of the invention include those described by formulas ad:

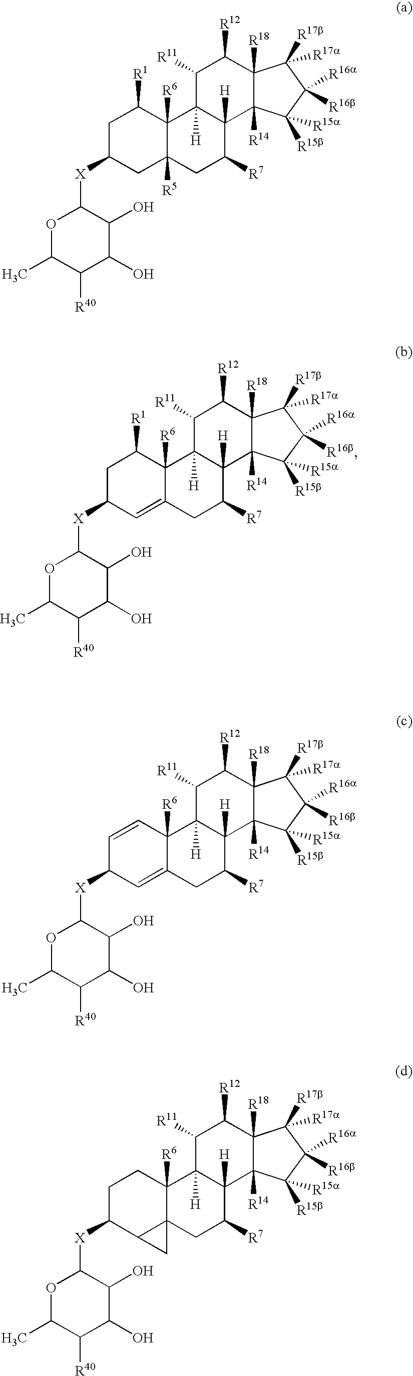

In formulas (a)-(d), X is NH or O; R⁴⁰ is F, Cl, CF₃, NH₂, NHR^{40A}, NR^{40B}R^{40C}, NHC(O)R^{40D}, NHC(S)R^{40E}, NHC(O)OR^{40F}, NHC(S)OR^{40G}, NHC(O)NHR^{40H}, NHC(S)NHR^{40I}, NHC(O)SR^{40J}, NHC(S)SR^{40K}, or NHS(O)₂R^{40L}; each of R^{40A}, R^{40B}, R^{40C}, R^{40D}, R^{40E}, R^{40F}, R^{40G}, R^{40H}, R^{40I}, R^{40J}, R^{40K}, and R^{40L} is, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{40B} and R^{40C} combine to form a C₂₋₄ heterocyclyl containing at least one nitrogen atom; each of R¹, R⁵, R⁷, R¹¹, and R¹² is, independently, H; OH, OR A, or OC(O)R A, where R^{1A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R⁶ is CH₃, CH₂OR^{6A}, or CH₂OCOR^{6A}, where R^{6A} is H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R¹⁴ is OH, Cl, OR^{14A}, or OC(O)R^{14A}, where R^{14A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R¹⁴, R^{15β}, and the carbons they are bonded to together represent an epoxide; each of R^{15α} and R^{15β} is, independently, H, OH, OR^{15A}, or OC(O)R^{15A}, where R^{15A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{15α} and R^{15β} together are ═O; each of R^{16α} and R^{16β} is, independently, H, OH, OR^{16A}, or OC(O)R^{16A}, where R^{16A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{16α} and R^{16β} together are ═O; R¹⁷ is

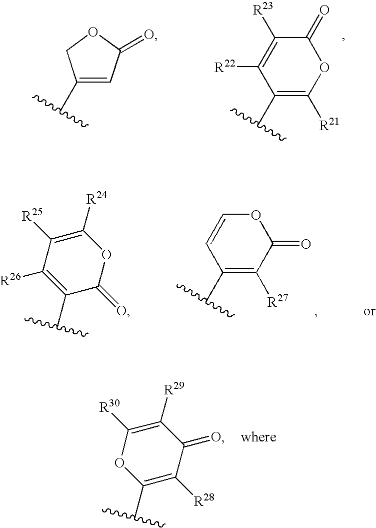

each of R²¹, R²², R²³, R²⁴, R²⁵, R²⁶, R²⁷, R²⁸, R²⁹, and R³⁰ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; R^{17α} is H or OH; and R¹⁸ is CH₃, CH₂OR^{18A}, or CH₂OCOR^{18A}, where R^{18A} is H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₁₃₋₁₀ alkheterocyclyl, or C₁₋₇ heteroalkyl.

### Synthesis

Many 3-hydroxy bufadienolide or cardiolide steroids have been previously described, such as, for example, those described by Karnano et al., in J. Med. Chem. 45:5440-5447, 2002; Kamano et al., in J. Nat. Prod. 65:1001-1005, 2002; Nogawa et al., in J. Nat. Prod 64:1148-1152, 2001; and Qu et al., J. Steroid Biochem. Mol. Biol. 91:87-98.

In addition, several different routes to the preparation of bufadienolides have been described in the art, including Soncheimer et al., J. Am. Chem. Soc. 91:1228-1230, 1969; Stache et al., Tetrahedron Lett. 35:3033-3038, 1969; Pettit et al., Can. J. Chem. 47:2511, 1969; Pettit et al., J. Org. Chem. 35:1367-9, 1970; Tsay et al., Heterocycles 12:1397-1402, 1979; Sen et al., J. Chem. Soc. Chem. Comm. 66:1213-1214, 1982; Wiesner et al., Helv. Chim. Acta 66:2632-2641, 1983; Weisner & Tsai, Pure and Appl. Chem. 53:799-810, 1986, and U.S. Pat. Nos. 4,001,402; 4,102,884; 4,175,078; 4,242,332; and 4,380,624.

A compound of the present invention, where R¹⁷ is a substituted 2H-pyran-5-yl-2-one moiety, can be prepared as shown in Scheme 1. Using the method of Stille (Angew. Chem. Int. Ed. Engl. 25:508, 1986), a compound of formula VI, where each of R²¹, R²², and R²³ is, independently, H, optionally substituted Clot alkyl, optionally substituted C₁₋₄ alkaryl, or optionally substituted C₃₋₈ cycloalkyl is prepared by reacting a compound of formula V with two equivalents of N-bromosuccinimide in CCl₄ in the presence of benzoyl peroxide (BPO). Using the method of Liu and Meinwald (J. Org. Chem. 61:6693-99, 1996), a compound of formula VI can be stannylated with hexamethyldistannane in the presence of a catalytic amount of Pd(PPh₃)₄ to produce a compound of formula VII, which can then be coupled to a steroid enol triflate, such as, for example, compound 102, to produce, after catalytic hydrogenation, a compound of formula VIII.

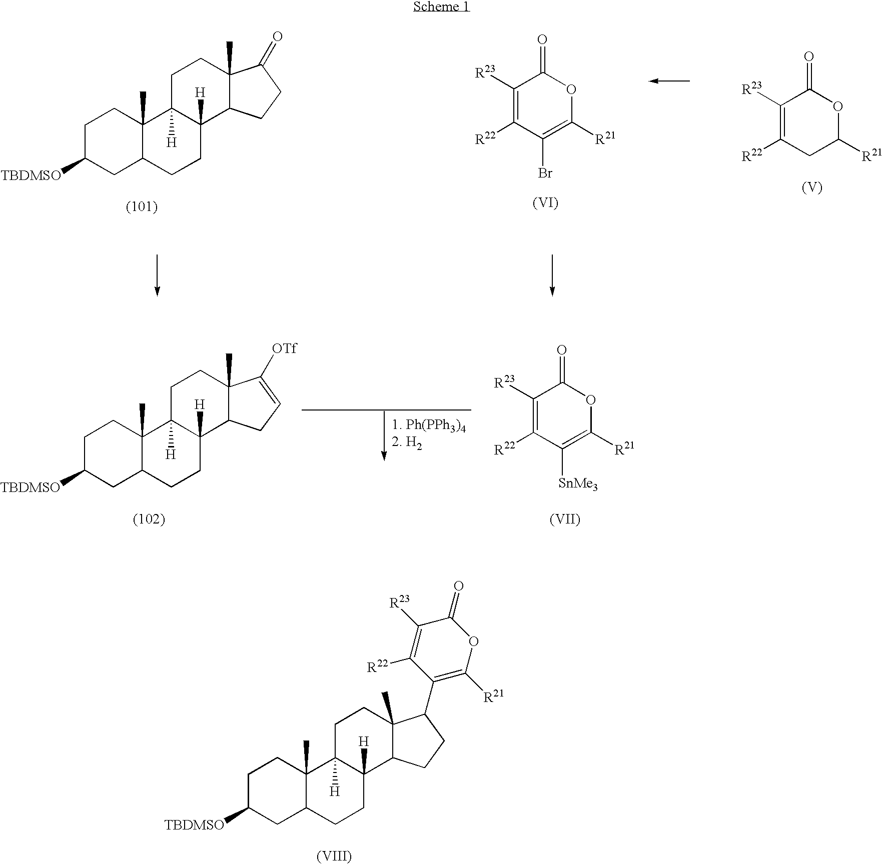

As shown in Scheme 2, a compound of formula VIII can be transformed to a compound of formula IX by photolysis in the presence of iodobenzene dichloride followed by treatment of the intermediate chloride with AgClG₄ (see Breslow et al., J. Am. Chem. Soc. 99:905, 1977 and Donovan et al., Tet. Lett. 35:3287-90, 1979). Treating the compound of formula IX with N-iodosuccinimide and reducing the resulting iodohydrin with Urishibara Ni-A produces a compound of formula X (see Karnano and Pettit, J. Am. Chem. Soc., 94(24):8592-3, 1972). Deprotection of the silylated 3-hydroxy group with potassium fluoride, followed by oxidation (e.g. with pyridinium chlorochromate or chromium trioxide), yields a ketone at the 3-position. Bromination at the 4-position with N-bromosuccinimide, followed by dehalogenation under basic conditions (e.g. refluxing collidine) produces a compound of formula XI. The hydroxyl at the 14-position can be optionally protected if subsequent steps require this. The keto group at the 3 position is reduced with a reagent such as, for example, lithium tri-tert-bitoxyaluminum hydride or lithium borohydride, to produce a compound of formula XII, which can be subsequently refunctionalized at the C-3 hydroxyl to produce a compound of formula XIII or XIV.

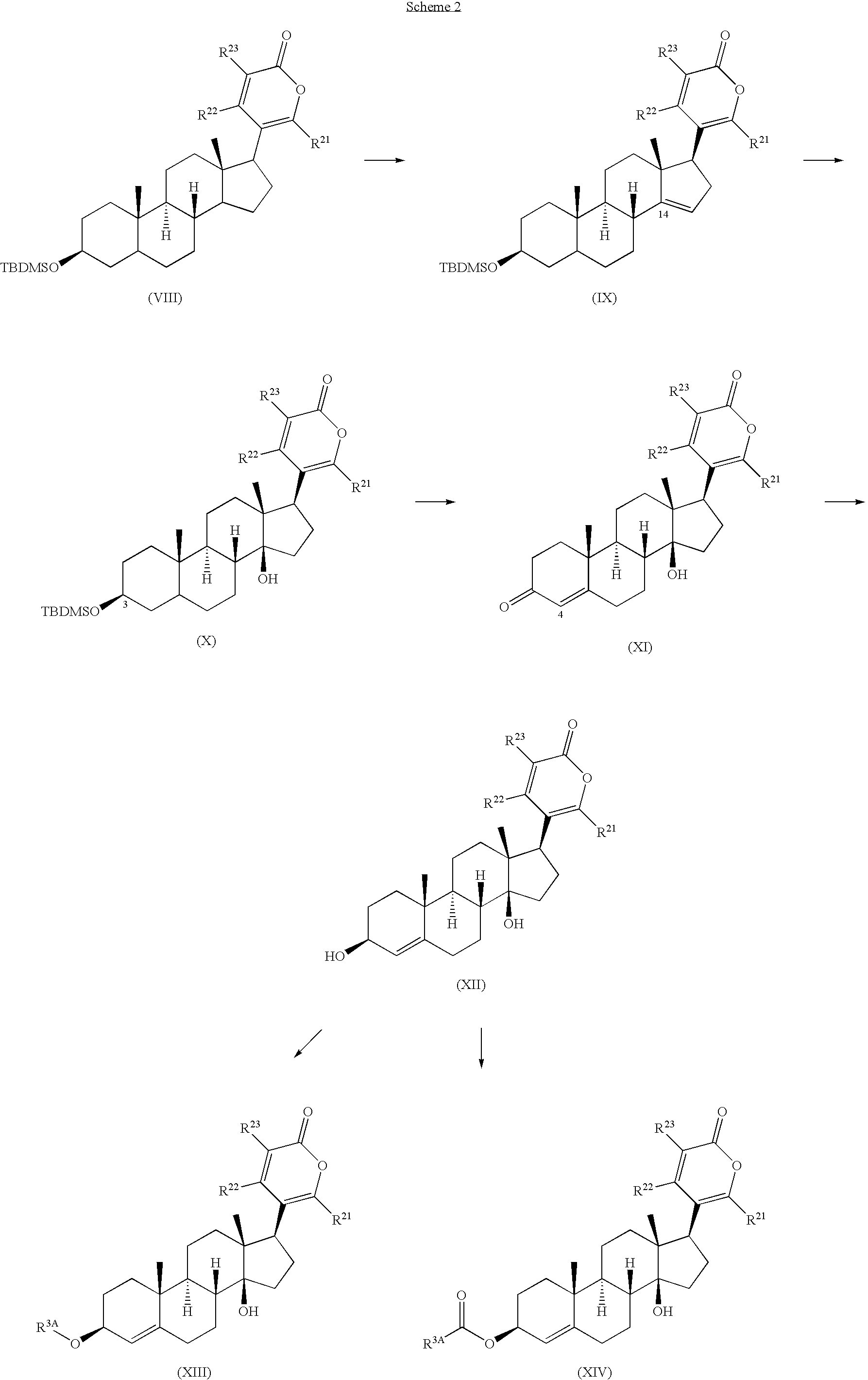

As shown in Scheme 3, chemistry analogous to that presented in Scheme 1 and described previously (see Stille, vide supra) for the transformation of a compound of formula V to a compound of formula VII can be used to produce a compound of formula XVI from a compound of formula XV, where each of R²⁴, R²⁵, and R²⁶ is, independently, H, optionally substituted C₁₋₆ alkyl, optionally substituted Cog alkaryl, or optionally substituted C₃₋₈ cycloalkyl. By chemistry analogous to that described above for the transformation of a compound of formula VII to a compound of formula XII, a compound of formula XVI can be taken on to produce a compound of formula XVII, where R¹⁷ is an optionally substituted 2H-pyran-3-yl-2-one moiety. As before, refunctionalization of the hydroxyl group at the 3-position can give a compound of formula XVIII or XIX.

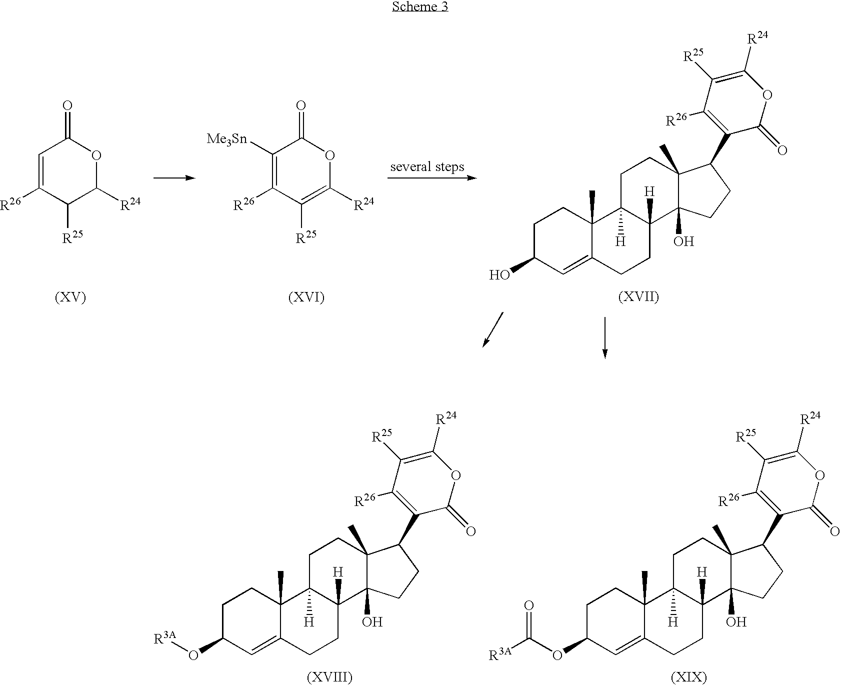

Bufadienolides in which R¹⁷ is a substituted 2H-pyran-4-yl-2-one moiety can be prepared as shown in Scheme 4 by a known procedure (see, for example, Wiesner et al., in Helv. Chim. Acta 65:2049-2060, 1982; Wiesner and Tsai, Pure & Appl. Chem. 58(5):799-810, 1986). Accordingly, a lithiated furan of formula XX, where R²⁷ is H, optionally substituted C₁₋₆ alkyl, optionally substituted C₁₋₄ alkaryl, or optionally substituted C₃₋₈ cycloalkyl, is reacted with compound 103 to produce a compound of formula XXI. Acetylation of the alcohol and allylic rearrangement in refluxing acetone in the presence of a base, such as, for example, calcium carbonate, produces, after the concomitant hydrolysis of the transposed acetate, a compound of formula XXII. Hydrogenation of the C16-C17 double bond is followed by deprotection of the acetal group and sodium borohydride reduction of the resulting aldehyde produces a compound; of formula XXIII. Treatment with mchloroperbenzoic acid gives a 2,5-dihydroxy dihydrofuran intermediate, which immediately rearranges to a compound of formula XXIV. Protection of bemiacetal hydroxyl as the acetate, elimination of the C₁₋₅ hydroxyl by treatment with thionyl chloride and pyridine, and removal of the acetyl protecting group by saponification provides a compound of formula XXV. Oxidation of the hemiacetal group to a lactone with chromic acid and reduction of the ketone with zinc borohydride gives a hydroxylactone of formula XXVI. Mesylation of the hydroxyl group followed by elimination yields a compound of formula XXVII. A hydroxyl group is introduced into the 14-position, as previously described, by treatment with N-iodosuccinimide and reduction of the resulting iodohydrin with Urishibara Ni-A. The benzyl protecting group at C3 is removed via hydrogenation, followed by oxidation (e.g. with pyridinium chlorochromate or chromium trioxide) to provide a ketone at the 3-position. As described before for the synthesis of a compound of formula XII, bromination, dehalogenation, and reduction produces a compound of formula XXVIII, which can be re-functionalized at the 3-position as previously described.

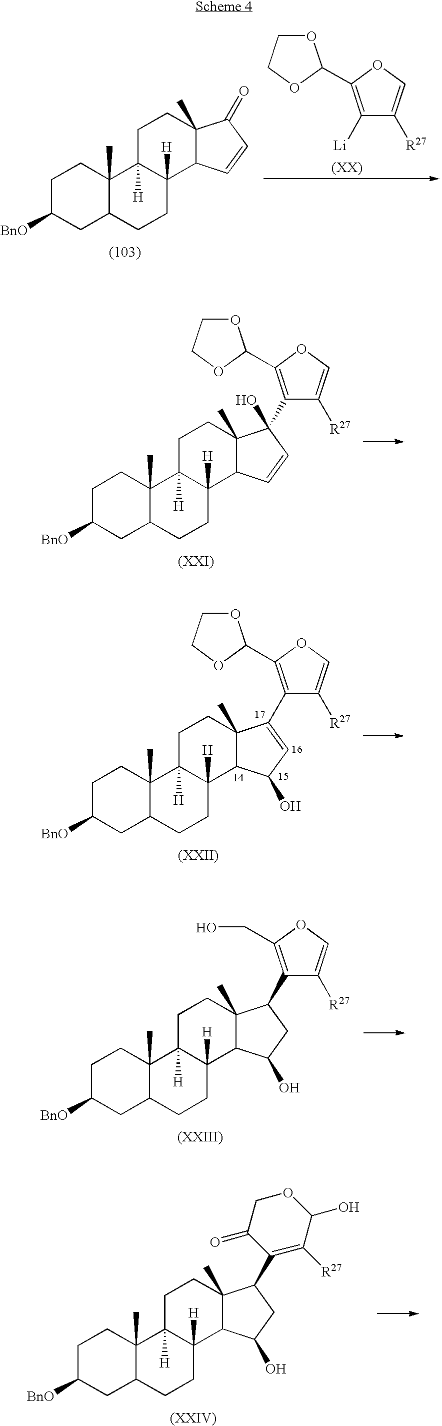

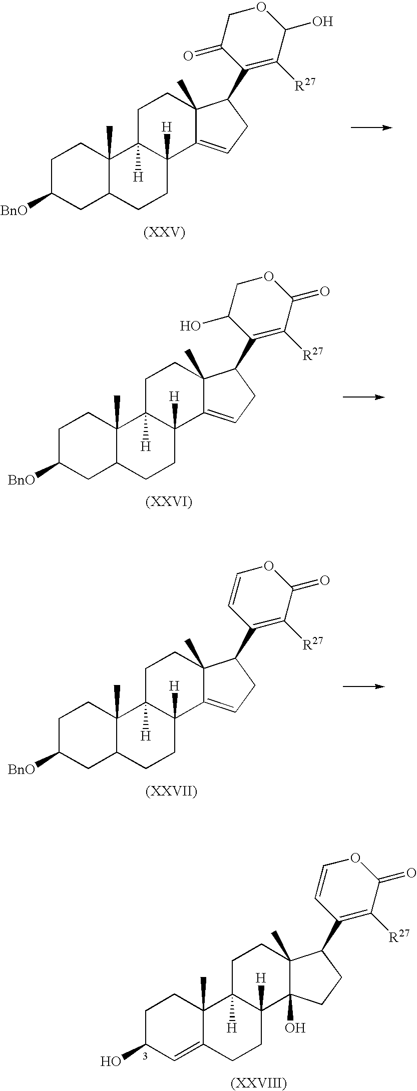

Bufadienolides in which R¹⁷ is a substituted 4H-pyran-2-yl-4-one moiety can be prepared as shown in Scheme 5. Accordingly, compound 103 is reacted with 2-lithiofuran to provide a compound of: formula XXX. Acetylation, allylic rearrangement, and hydrogenation, as previousty described for a compound of formula XXI, followed by reacetylation, provides a compound of formula XXXI. Treatment of the furan ring with N-bromosuccinimide, followed by oxidation with KMnO₄/NaIO₄ in the presence of K₂CO₃ yields a carboxylic acid at the C17 position, which can be activated by treatment with 1,1′-carbonyldiimidazole to provide a compound of formula XXXII. Reaction with the potassium enolate of formula XXXIII yields, after acidic quenching, γ-pyrone of formula XXXIV. Compounds of formula XXXIII can be prepared by reacting compounds of formula XXXIIIa with lithium diisopropylamide or lithium hexamethyldisilazide under appropriate conditions. Removal of the acetyl group, mesylation, elimination, and introduction of a hydroxyl group into the 14-position by treatment with N-iodosuccinimide and reducing the resulting iodohydrin with Urishibara Ni-A, as previously described, produces a compound of formula XXXV. The benzyl protecting group at C3 is removed via hydrogenation, followed by oxidation (e.g. with pyridinium chlorochromate or chromium trioxide) to provide a ketone at the 3-position. As described before for the synthesis of a compound of formula XII, bromination, dehalogenation, and reduction produces a compound of formula XXXVI, which can be re-functionalized at the 3-position.

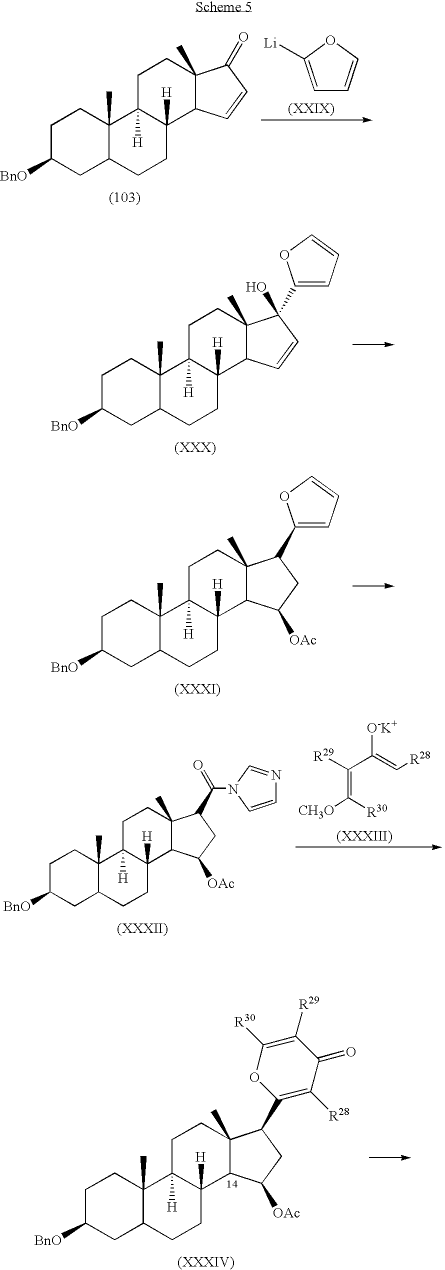

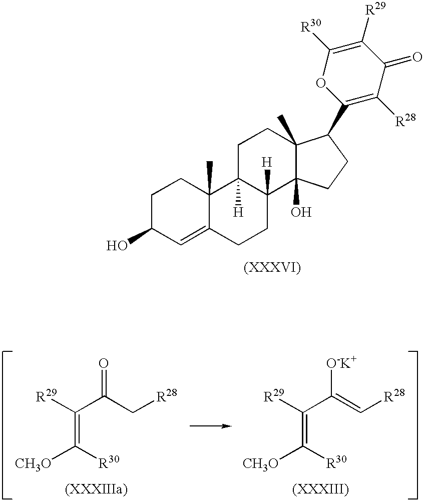

As shown in Scheme 6, for any of the compounds of the described herein that are substituted at the 17-position with a 2H-pyran-2-one moiety, the 17 position can be further functionalized by oxidation to produce a compound of formula XXXIX, where R^{17α}, is OH (see Saito et al., Chem. Pharm. Bull. 18:69, 1970 and Templeton et al., Steroids 65:379, 2000).

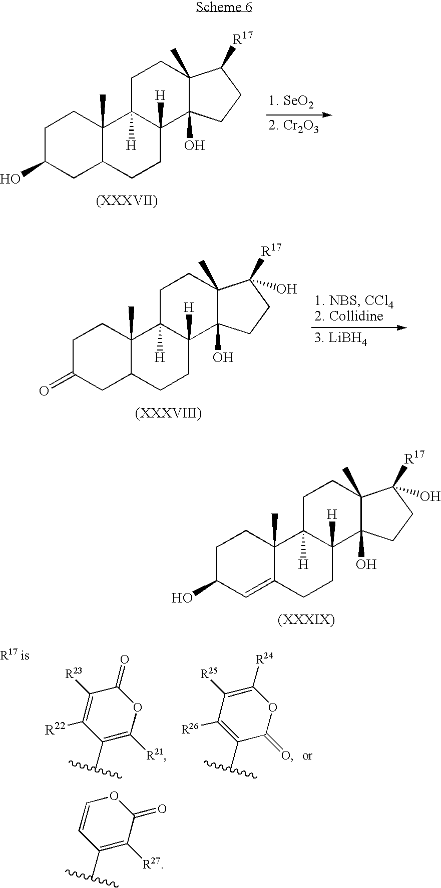

Saccharide derivatives can be prepared as described in the examples, or by using any of reactions 1-3 below. Each of these reaction schemes can be applied to any other corresponding 3-hydroxy or 3-amino cardiolide or bufadienolide described herein to produce the corresponding saccharide. Derivatized saccharides can

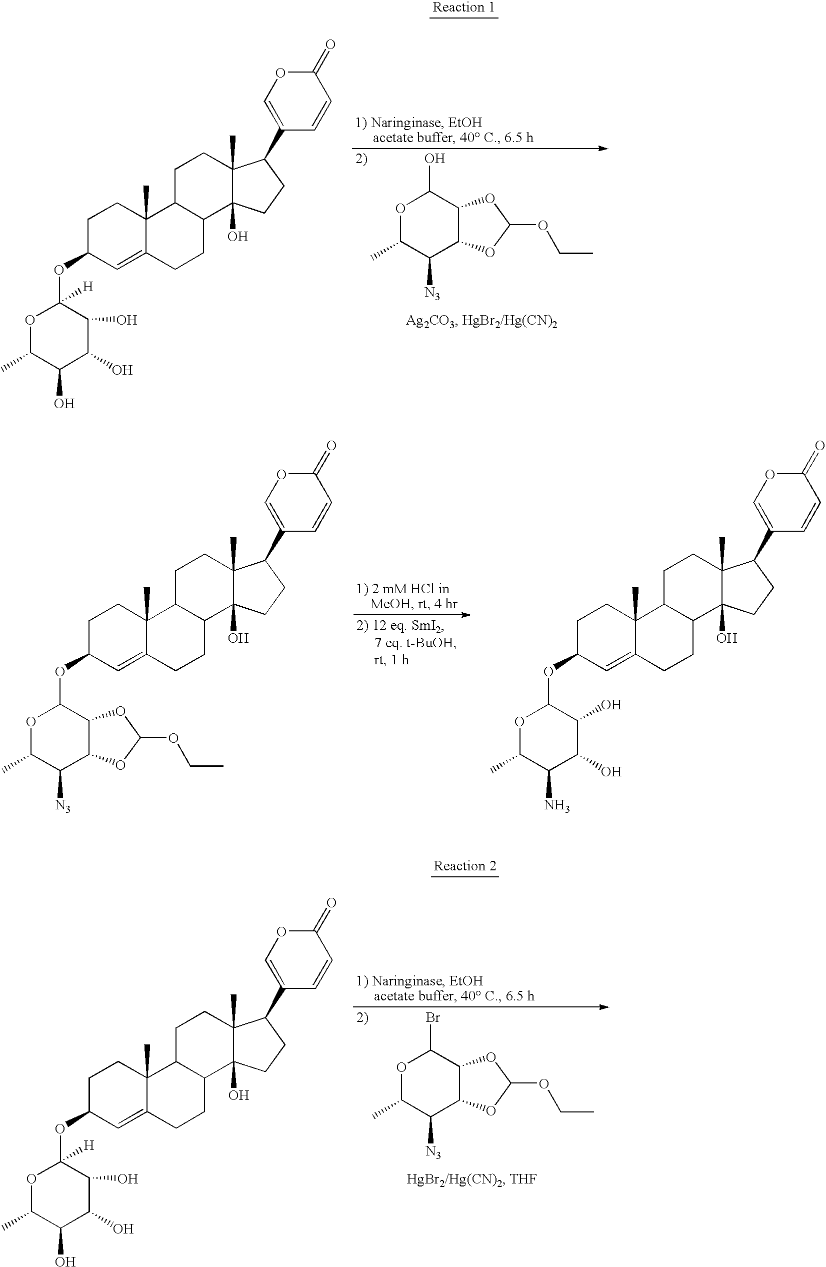

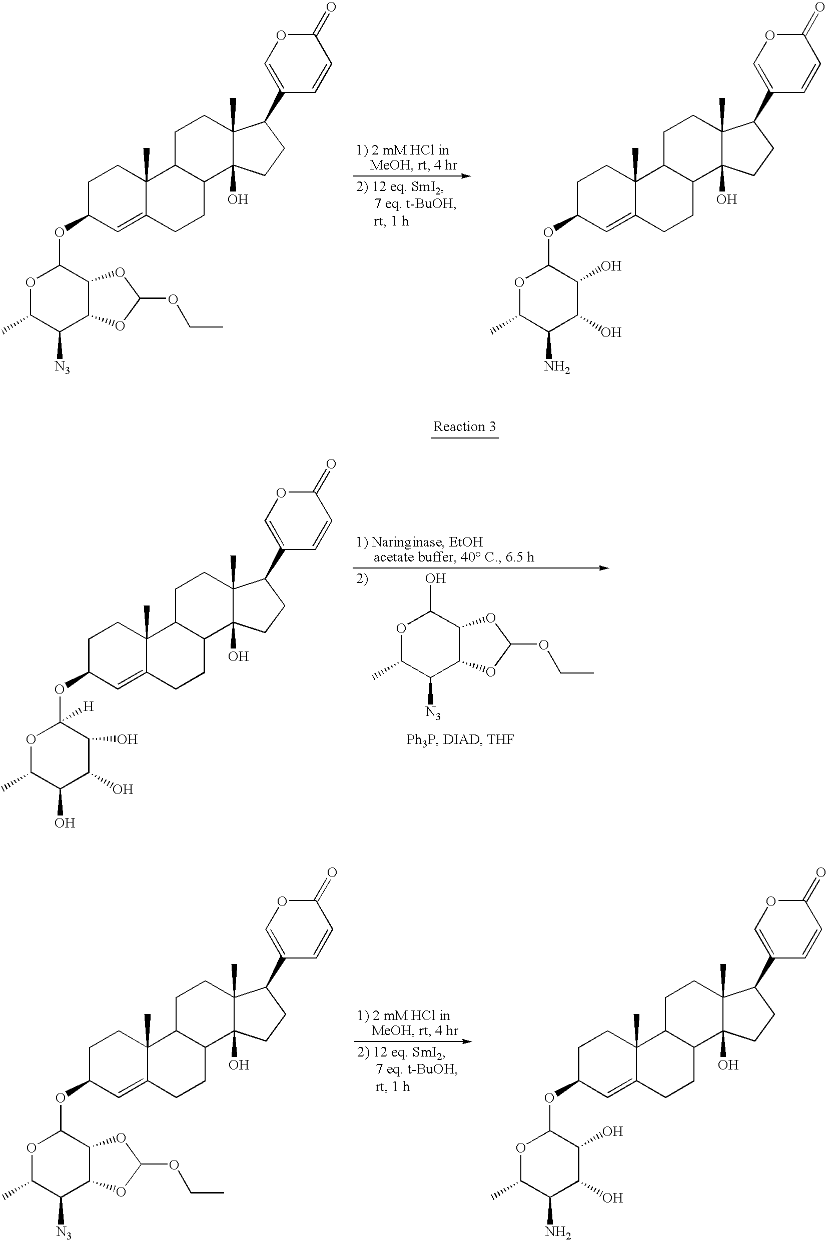

employed in the same fashion to produce a variety of cardiolide and bufadienolide analogs.

### EXAMPLES

The following examples are put forth so as to provide those of ordinary skill in the art with a complete disclosure and description of how the methods and compounds claimed herein are performed, made, and evaluated, and are intended to be purely exemplary of the invention and are not intended to limit the scope of what the inventors regard as their invention.

The exemplary HIF-1-modulating compounds used in following studies are referred to as BNC1 and BNC4. Compounds of the invention include BP244 and BP228, shown below.

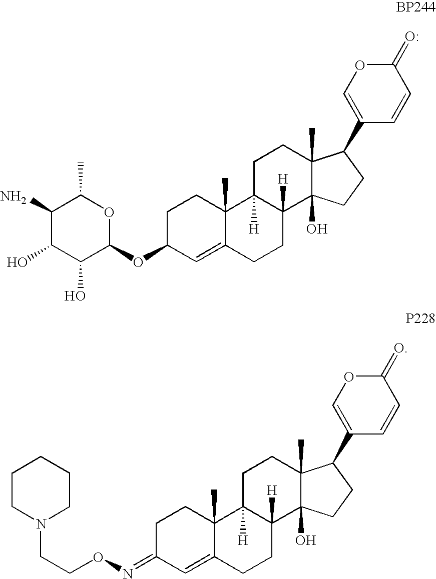

BNC1 is ouabain or G-Strophanthin (STRODIVAL®), which has been used for treating myocardial infarction. It is a colorless crystal with predicted IC₅₀ of about 0.06-0.35 μg/mL and max. plasma concentration of about 0.03 μg/mL. According to the literature, its plasma half-life in human is about 20 hours, with a range of between 5-50 hours. Its common formulation is injectable. The typical dose for current indication (i.v.) is about 0.25 mg, up to 0.5 mg/day.

BNC4 is proscillaridin (TALUSIN®), which has been approved for treating chronic cardiac insufficiency in Europe. It is a colorless crystal with predicted IC₅₀ of about 0.01-0.06 μg/mL and max. plasma concentration of about 0.1 μg/mL. According to the literature, its plasma half-life in human is about 40 hours. Its common available formulation is a tablet of 0.25 or 0.5 mg. The typical dose for current indication (p.o.) is about 1.5 mg/day.

### Example 1

### Cardiac Glycoside Compounds Inhibits HIF-1α Expression

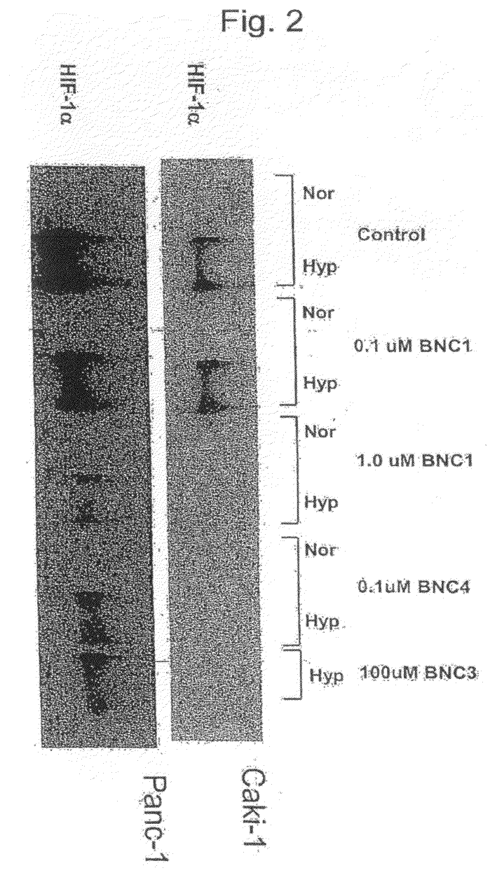

The ability of BNC1 and BNC4 to inhibit hypoxia-mediated HIF1α induction in human tumor cells was investigated. FIG. 2 shows the result of immunoblotting for HIF-1α, HIF-1β and β-actin (control) expression in Caki-1 or Panc-1 cells treated with BNC1 or BNC4 under hypoxia. The results indicate that BNC4 is about 10 times more potent than BNC1 in inhibiting HIF-1α expression.

### Example 2

### BNC4 Inhibits HIF-1α Induced Under Normoxia by PHD Inhibitor

To study the mechanism of BNC4 inhibition of HIF-1α, the ability of BNC1 or BNC4 to inhibit HIF-1α expression induced by a PHD inhibitor, L-mimosone, was investigated under normoxia condition.

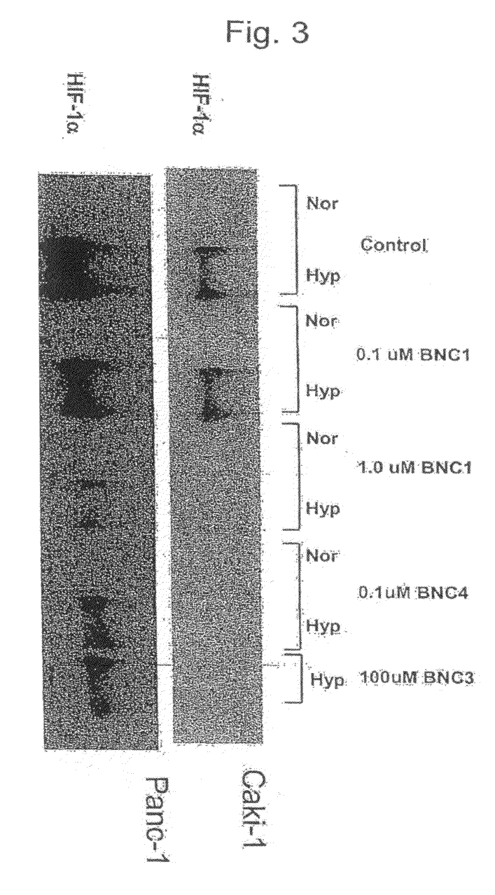

In the experiment represented in FIG. 3, Hep3B cells were grown under normoxia, but were also treated as indicated with 200 μM L-mimosone for 18 hours in the presence or absence of BNC1 or BNC4. Abundance of HIF1α and β-actin was determined by Western blotting.

The results indicate that L-mimosone induced HIF-1α accumulation under normoxia condition, and addition of BNC4 eliminated HIF-1α accumulation by L-mimosone. At the low concentration tested, BNC1 did not appear to have an effect on HIF-1α accumulation in this experiment. While not wishing to be bound by any particular theory, the fact that BNC4 can inhibit HIF-1α induced under normoxia by PHD inhibitor indicates that the site of action by BNC4 probably lies downstream of prolyl-hydroxylation.

### Example 3

### Preparation of 3-Oximethers and 3-Amino Derivatives of Scillarenin

Synthesis of Scillarenin

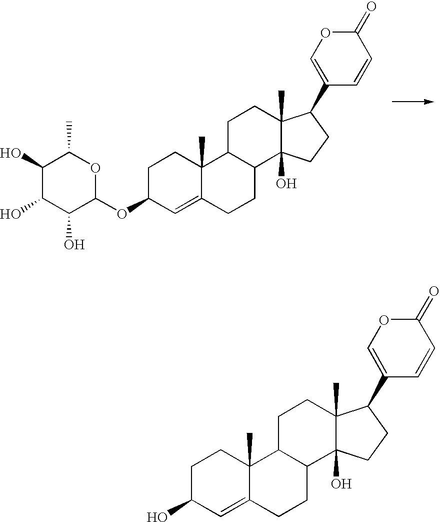

A solution (partial suspension) of proscillaridin (66.3 mg, 0.125 mmol) and naringinase (23.2 mg) in EtOH (1.25 mL)-0.02 M acetate buffer (pH 4.0, 3.75 mL) was incubated at 40° C. for 6.5 h. After addition of EtOH (30 mL), the whole mixture was concentrated under reduced pressure. The resulting residue was purified by column chromatography (SiO₂, 10 g, n-hexanes-EtOAc (1:1)) to furnish scillarenin (48 mg).

### Synthesis of Scillarenon

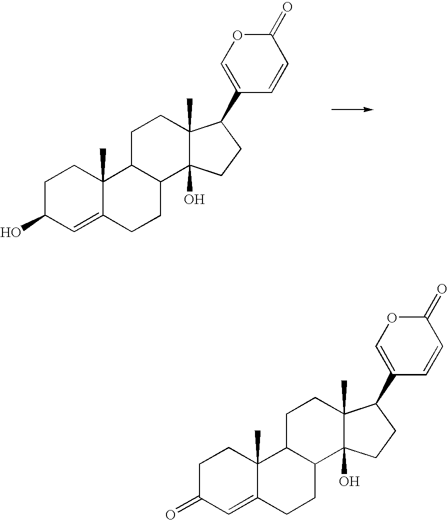

700 mg (1.82 mmole) of scillarenin was dissolved in 30 mL of dry dichloromethane and 1.4 g of powdered molecular sieve and 1.57 g (7.28 mmole) of pyridinium chlorochromate were added. The mixture was stirred under a nitrogen atmosphere at room temperature overnight. The dark mixture was filtered through a pad of Celite and concentrated. The crude mixture was purified by flash chromatography to yield 604 mg (86%) of the desired ketone as a colorless solid.

### Synthesis of O-(2-Ethylpiperidino)-hydroxylamine

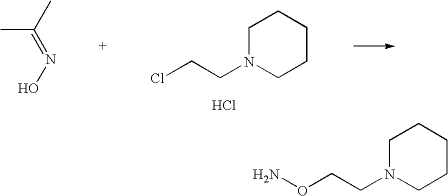

Sodium, 13.8 g (600 mmole), was dissolved in 450 mL of dry ethanol and 21.9 g (300 mmole) of acetone oxime and 55.2 g (300 mmole) of piperidinoethylchloride hydrochloride were added and the mixture refluxed for 2 h. The mixture was concentrated to about ⅓ of its original volume. Water was added and the mixture was extracted with diethyl ether. The organic extracts were washed with water and dried over Na₂SO₄. After concentration in vacuo the residue was distilled under reduced pressure (bp 100° C. at 22 mbar) to yield 33.4 g (60%) of the acetone oximether. 15 g of this material was refluxed in 6 N HCl overnight. After cooling, the mixture was basified with NaOH-solution and extracted with diethyl ether. The organic extracts were dried, concentrated and the residue was distilled under reduced pressure (bp 101-106° C. at 18 mbar) to yield 2.7 g (23%) of the desired hydroxylanine derivative as a colorless liquid.

### Synthesis of 3-(O-(2-Ethylpiperidino))-scillarenone-oximether

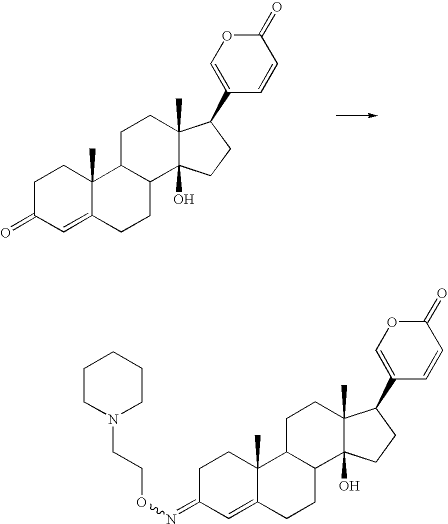

To a solution of 650 mg (1.7 mmole) of scillarenone in 50 mL of dry methanol were added 1.59 g (11.05 mmole) of O-(2-ethylpiperidino)-hydroxylamine and 3 mL of glacial acetic acid and the mixture was stirred at room temperature for 90 minutes. The mixture was diluted with ethyl acetate and washed with saturated NaHCO₃-solution and brine. The organic extracts were dried with Na₂SO4, solvent was evaporated under reduced pressure and the crude product was purified by flash chromatography to give 773 mg (85%) of the desired oximether as a colorless solid.

### Synthesis of 3-(O-methyl)-scillarenone-oximether

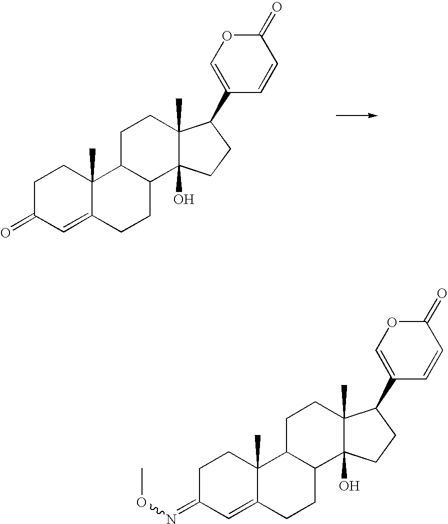

To a solution of 650 mg (1.7 mmole) of scillarenone in 50 mL of dry methanol were added 1420 mg (17 mmole) of O-methylhydroxylamine hydrochloride and 1283 mg (15.64 mmol) of sodium acetate and the mixture was stirred at room temperature for 3 hours. The mixture was diluted with ethyl acetate and washed with saturated NaHCO₃-solution and brine. The organic extracts were dried with Na₂SO₄, solvent was evaporated under reduced pressure and the crude product was purified by flash chromatography to give 88% of the desired oximether as a colorless solid.

Scillarenin 3-oximethers and 3-amino derivatives can be prepared as described below in Scheme 7.

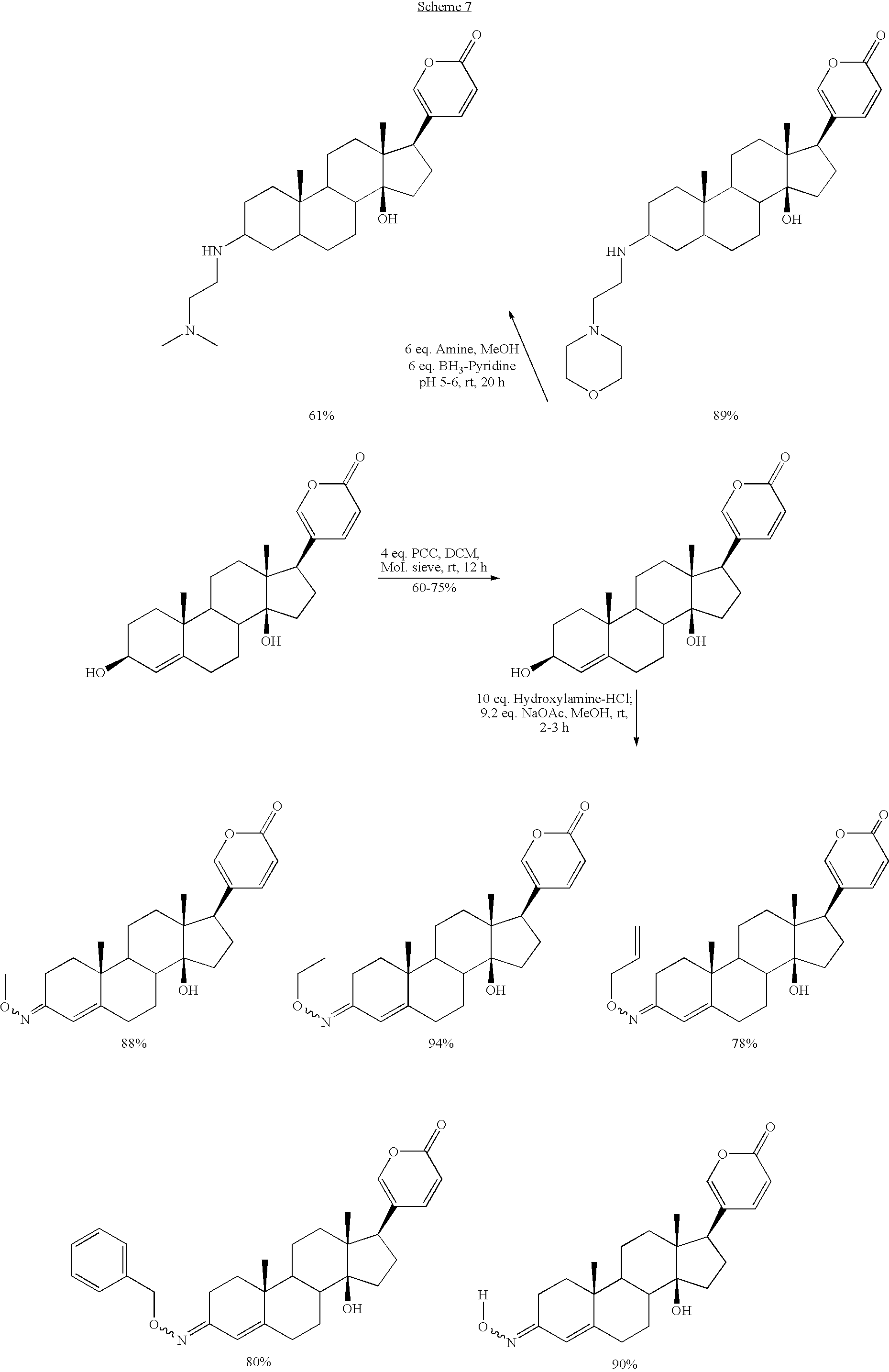

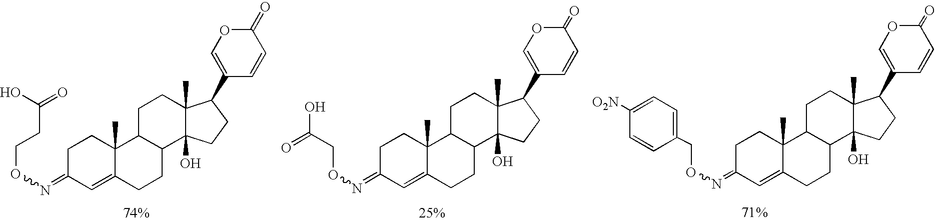

### Example 4

### Preparation of 3-Oximethers, 3-Hydrazone, and 3-Ether Derivatives of Scillarenin

Scillarenin 3-oximethers, 3-hydrazone, and 3-ether derivatives can be prepared as described below in Scheme 8.

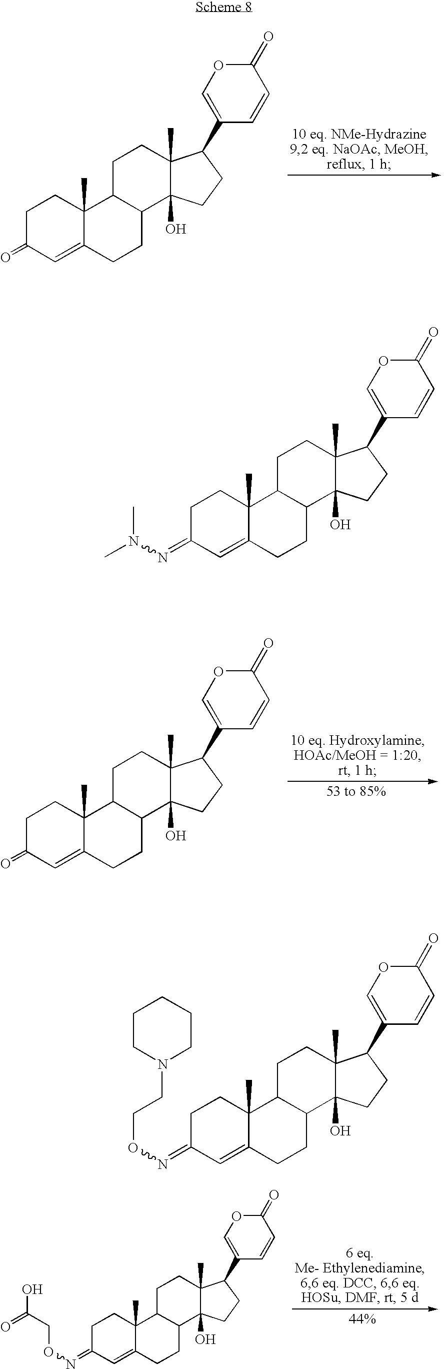

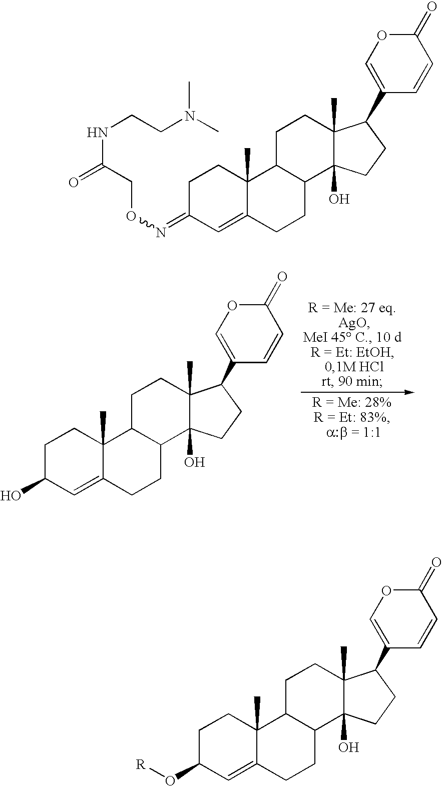

### Example 5

### Preparation of 3-Acyl Derivatives of Scillarenin

Scillarenin 3-acyl derivatives can be prepared as described below in Schemes 9a, 9b, and 9c.

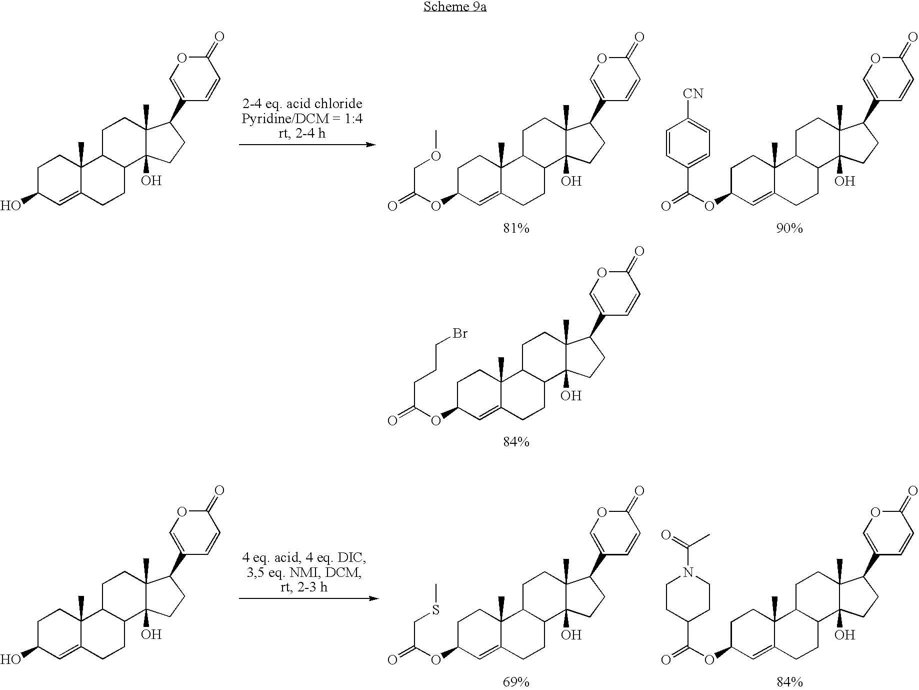

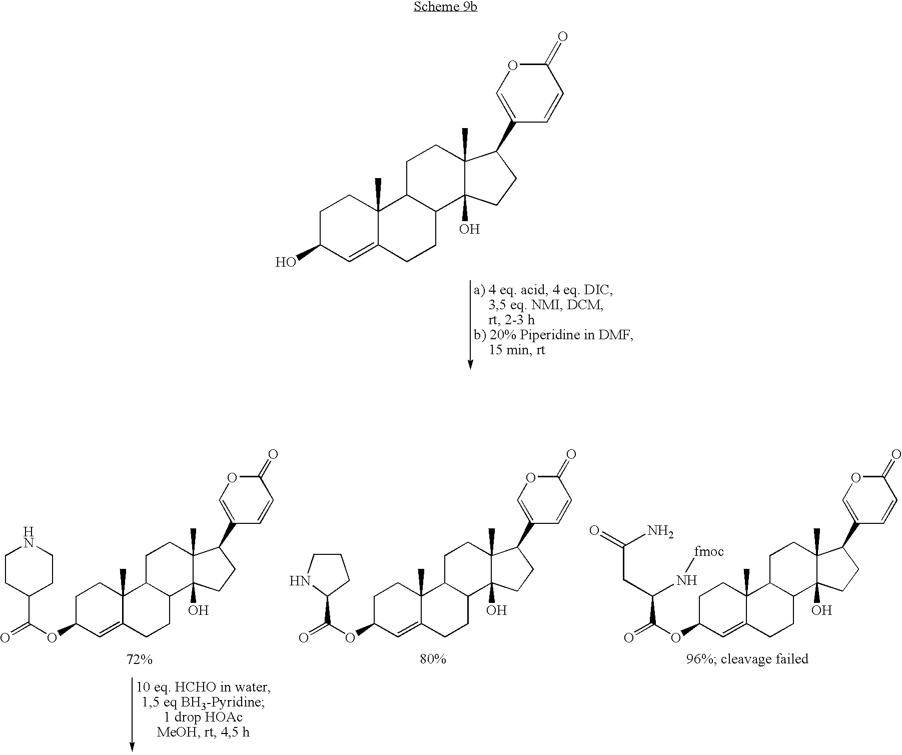

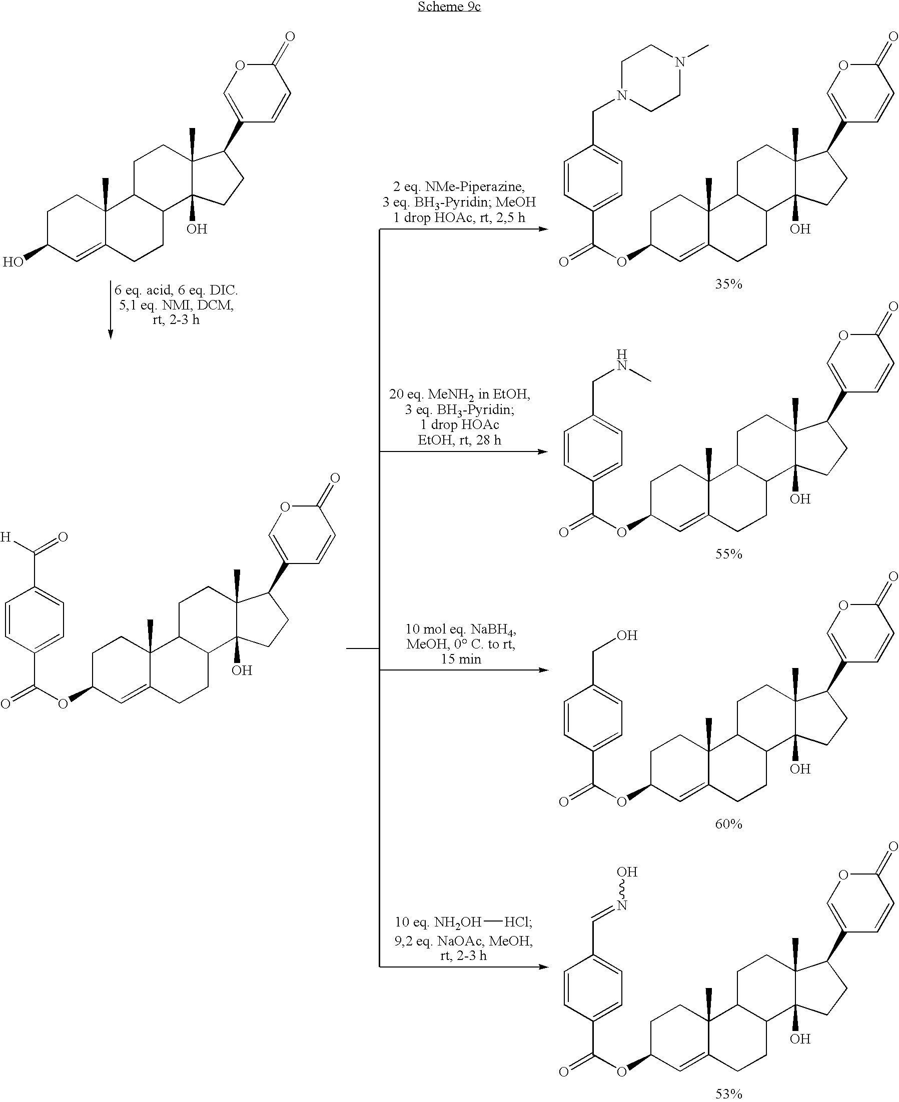

### Example 6

### Preparation of 3-Carbamoyl Derivatives of Scillarenin

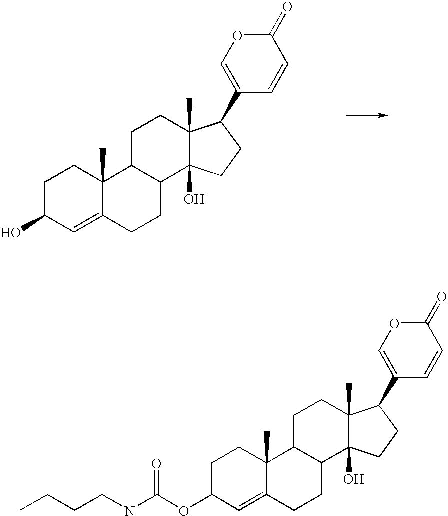

To a solution of 25 mg (0.065 mmole) of scillarenin in 0.5 mL of pyridine was added 18.8 mg (0.19 mmole) of butyl isocyanate and 6 mg (0.065 mmole) of CuCl and the mixture was stirred at room temperature until complete consumption of starting material was detected.

After 30 min the mixture was partitioned between ethyl acetate and water. The aqueous phase was extracted with ethyl acetate three times and the combined organic extracts were washed with 1 M HCl and brine. After drying over Na₂SO₄ and removal of solvent the crude product was purified by flash chromatography yielding 13.7 mg (44%) of the desired carbamate as a colorless solid.

Scillarenin 3-carbamoyl derivatives can be prepared as in Schemes 10a and

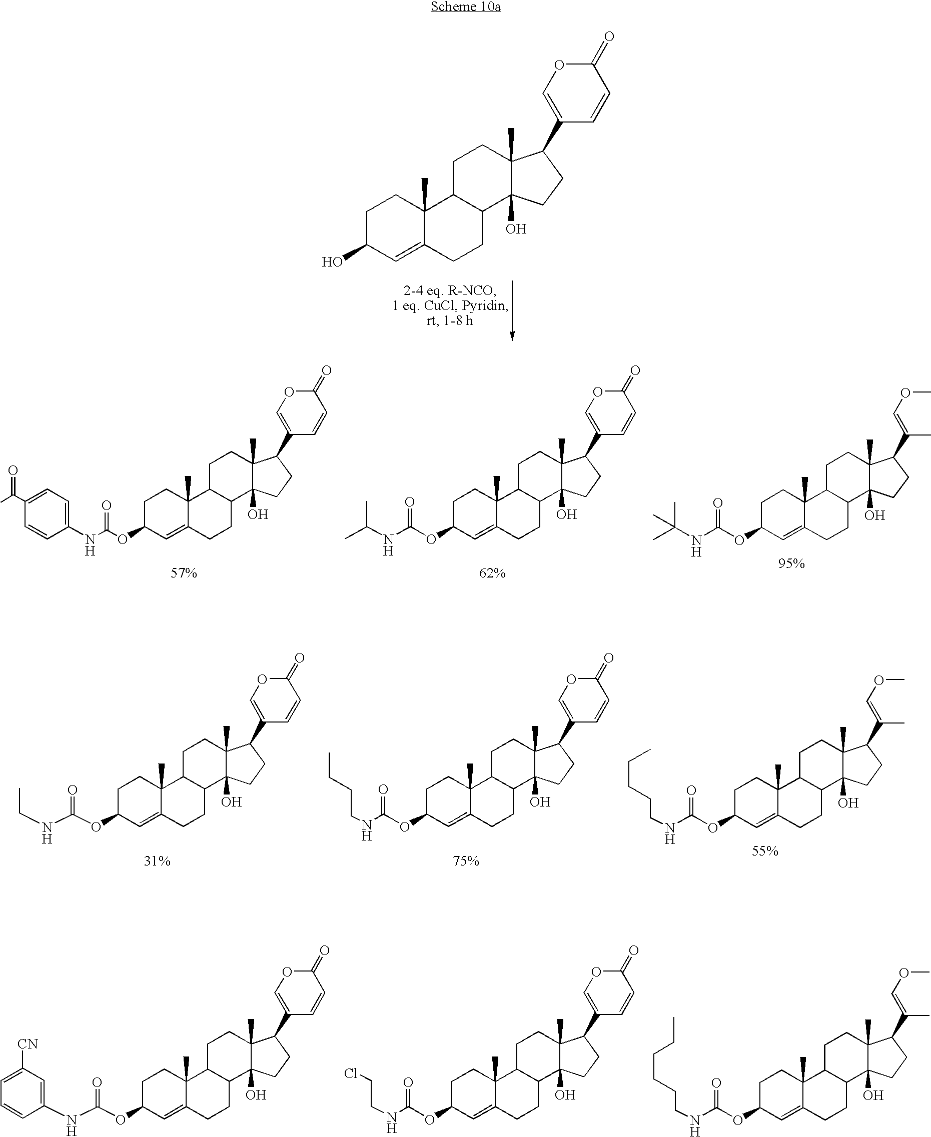

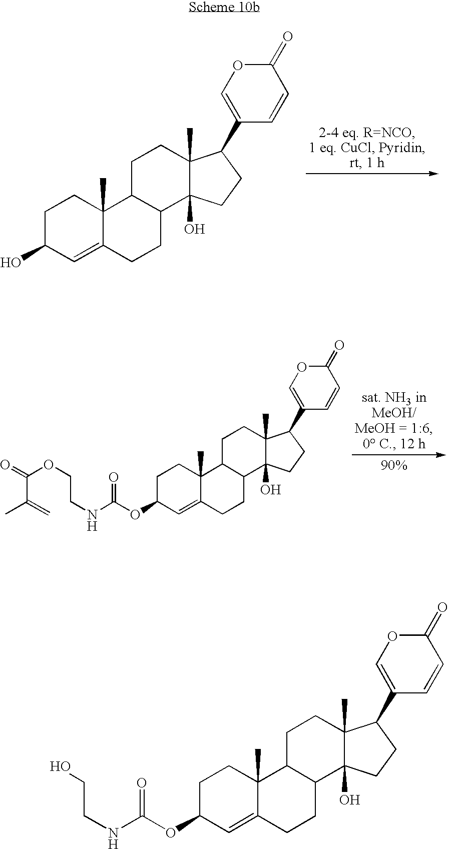

### Example 7

### Preparation of 3-Amino-Derivatives of Scillarenin

Scillarenin 3-amino derivatives can be prepared as described below in Scheme 11.

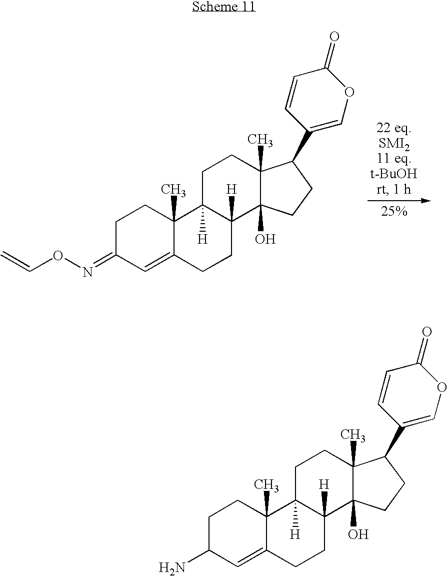

### Example 8

### Preparation of 3-O-Saccharide Derivatives

### Synthesis of 4′-Oxo-2′,3′-(O-ethoxymethyl)-proscillaridin

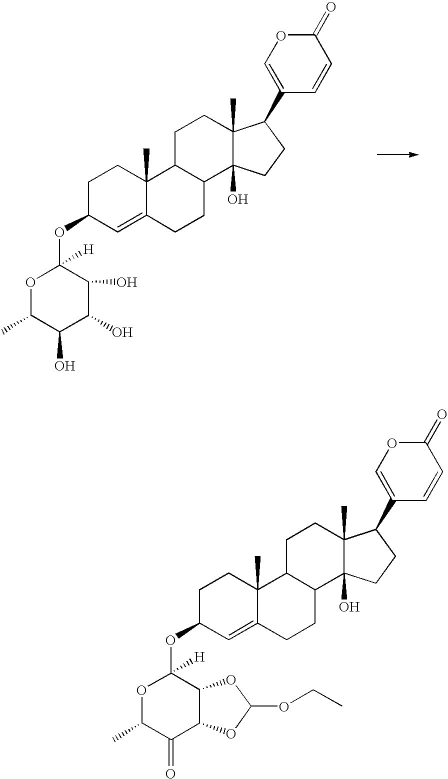

To a stirred solution of 1 g (1.9 mmole) of proscillaridin in 5 mL of dry tetrahydrofuran was added a crumb of p-TsOH and 1.34 mL (8.05 mmole) of triethyl orthoformate at room temperature. The organic layer was washed with water and dried over Na₂SO₄. Concentration and column chromatography yielded 740 mg (66%) of the 4′-hydroxy ortho ester a pale yellow solid. 704 mg (1.02 mmole) of this product was dissolved in 25 mL of dry dichloromethane. 1.05 g of powdered molecular sieve and 881 mg (4.08 mmole) of pyridiniumchloro chromate were added and the mixture stirred under a nitrogen atmosphere at room temperature overnight. The dark mixture was filtered through a pad of Celite and concentrated. The crude product was purified by flash chromatography to yield 246 mg (41%) of the desired ketone as a colorless solid.

### Synthesis of 4′-β-Hydroxy-2′,3′-(O-ethoxymethyl)-proscillaridin

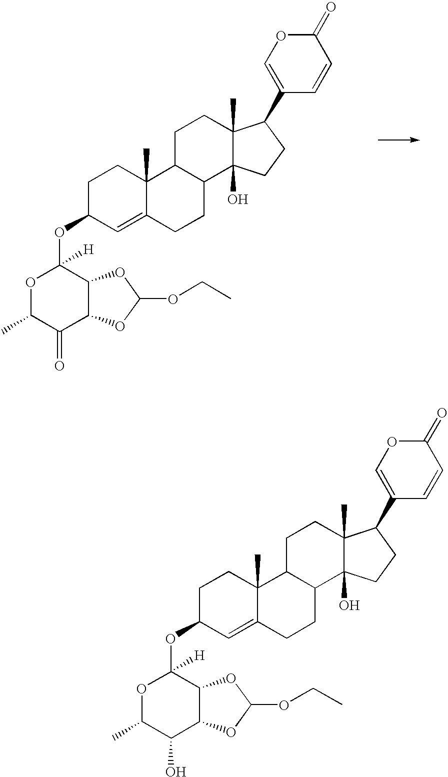

To a solution of 234 mg (0.4 mmole) of the starting ketone in 5 mL of dry methanol was added 110 mg (2.9 mmole) of sodium borohydride at 0° C. After complete addition the ice bath was removed and the mixture stirred was for another 15 minutes at room temperature. The mixture was diluted with ethyl acetate and washed with water. The organic phase was dried with Na₂SO₄, solvent evaporated to give crude alcohol (232 mg, 99%) which was used for the next step without further purification.

### Synthesis of 4′-β-Azido-2′,3′-(O-ethoxymethyl)-proscillaridin

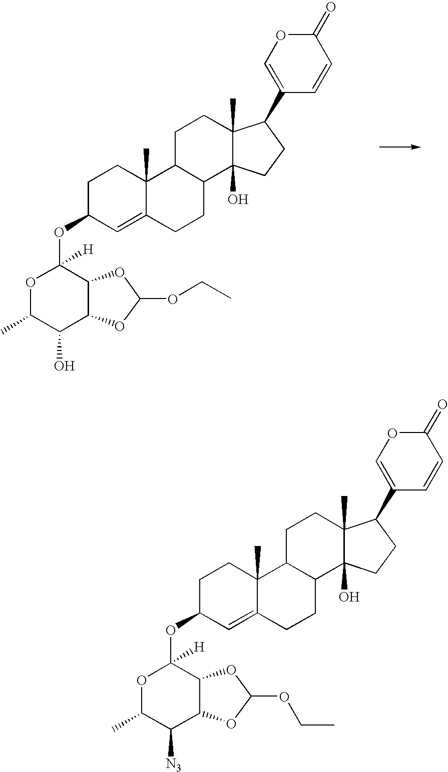

To a solution of 151 mg (0.264 mmole) of the starting alcohol in 2 mL of dry dichloromethane and 1.5 mL of dry pyridine was added 109 μl (0.66 mmole) of trifluoromethane sulfonic anhydride at −20° C. After complete addition the cooling bath was removed and replaced by an ice bath and the mixture was stirred for two more hours at the same temperature. The mixture was diluted with dichloromethane, transferred to a separatory funnel and washed with 1 molar HCl, followed by saturated NaHCO₃ solution and water. The organic phase was dried with Na₂SO₄ and concentrated. The crude triflate was dissolved in 2 mL of dry dimethylformamide, 59 mg (0.9 mmole) of sodium azide was added and the mixture was stirred at room temperature overnight. Water and dichloromethane were added and the organic layer was washed with water. The solvent was dried over Na₂SO₄ and evaporated to give crude residue which was purified by column chromatography yielding 84 mg (52%) of the desired azide as a colorless solid.

### Synthesis of 4′-β-Azido-proscillaridin

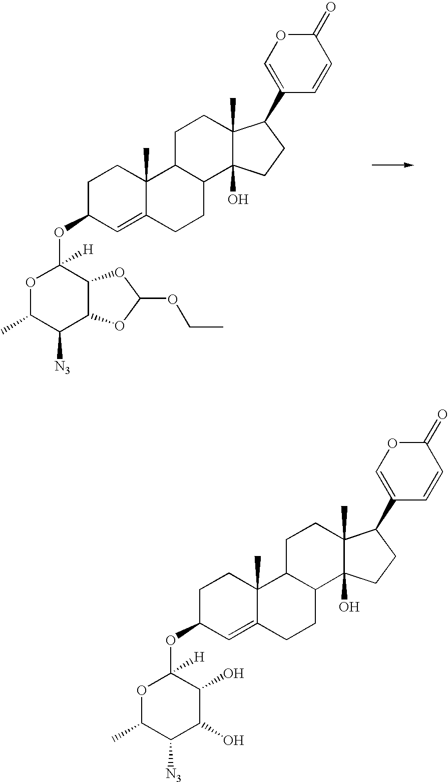

To a solution of 42 mg (0.069 mmole) of the protected azide in 0.8 mL of ethyl acetate was added 0.8 mL of 0.002 molar methanolic HCl and the mixture stirred for two hours at room temperature. The mixture was diluted with ethyl acetate and washed with water and brine. The organic phase was dried over Na₂SO₄, concentrated and the crude product was purified by column chromatography to yield 26 mg (69%) of the desired dihydroxy azide as a colorless solid.

### Synthesis of 4′-β-Amino-proscillaridin

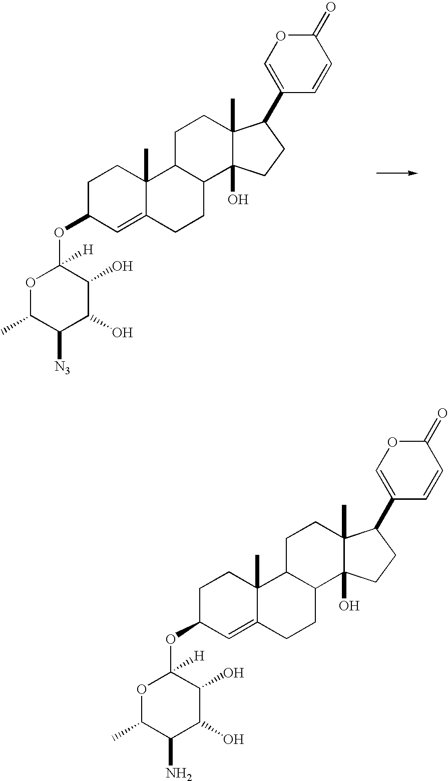

18 mg (0.033 mmole) of the starting azido steroid was charged with 3.6 mL mmole) of a 0.1 molar solution of SmI₂ in tetrahydrofuran under an argon atmosphere. The mixture was stirred at room temperature for 10 minutes, 14 μL of tert-butyl alcohol was added and stirring was continued for another 50-90 minutes. The mixture was hydrolyzed with saturated NaHCO₃ solution and extracted with ethyl acetate. The organic extracts were dried and concentrated in vacuo to give an yellow oil which was purified by flash chromatography. After purification 6.5 mg of amine (35%) was obtained of a colorless solid.

Scillarenin 3-O-saccharide derivatives can be prepared as described below in Schemes 12a, 12b, and 12c.

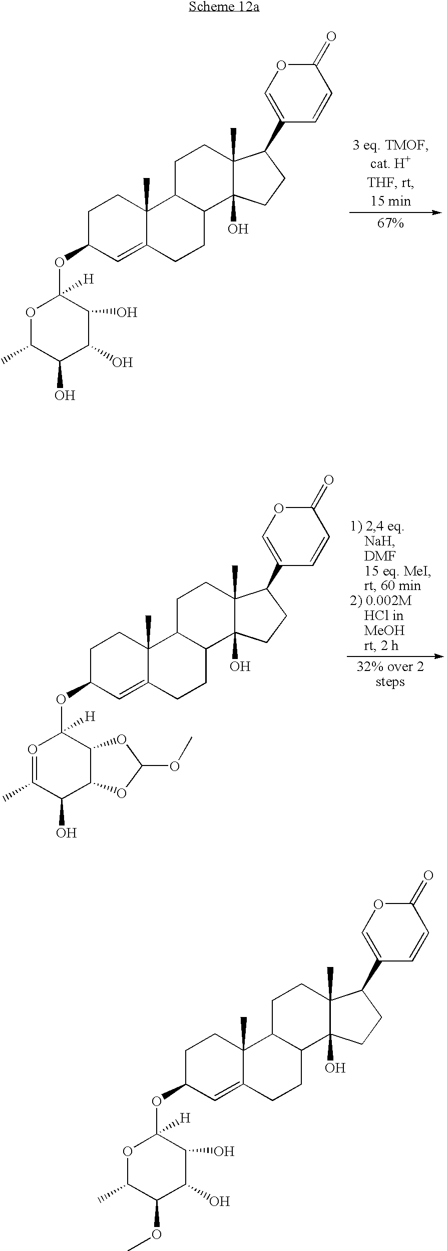

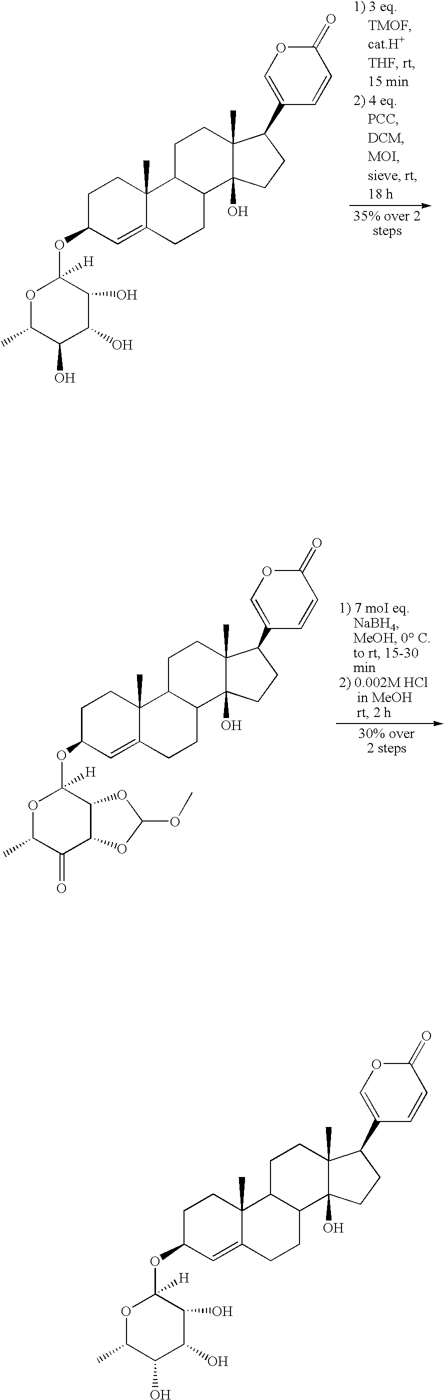

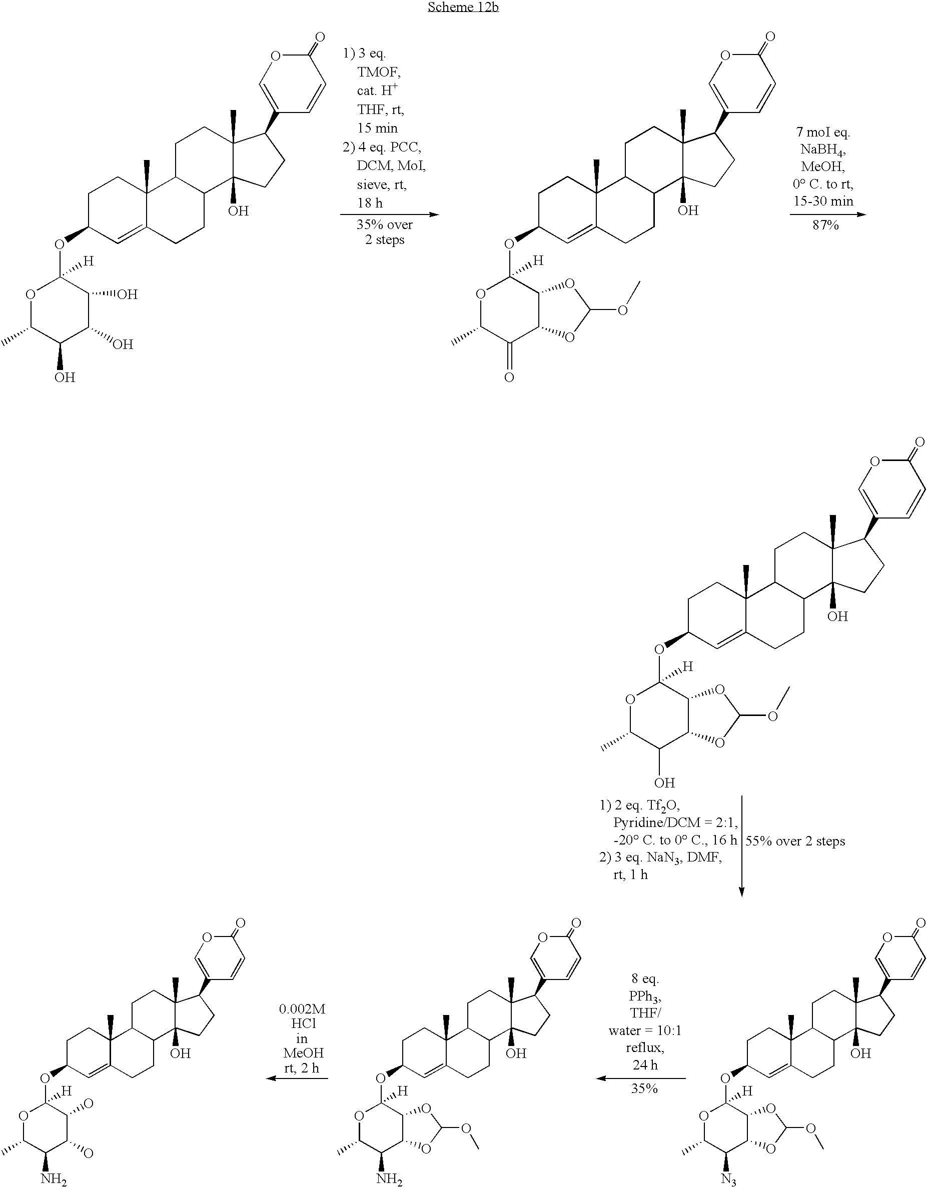

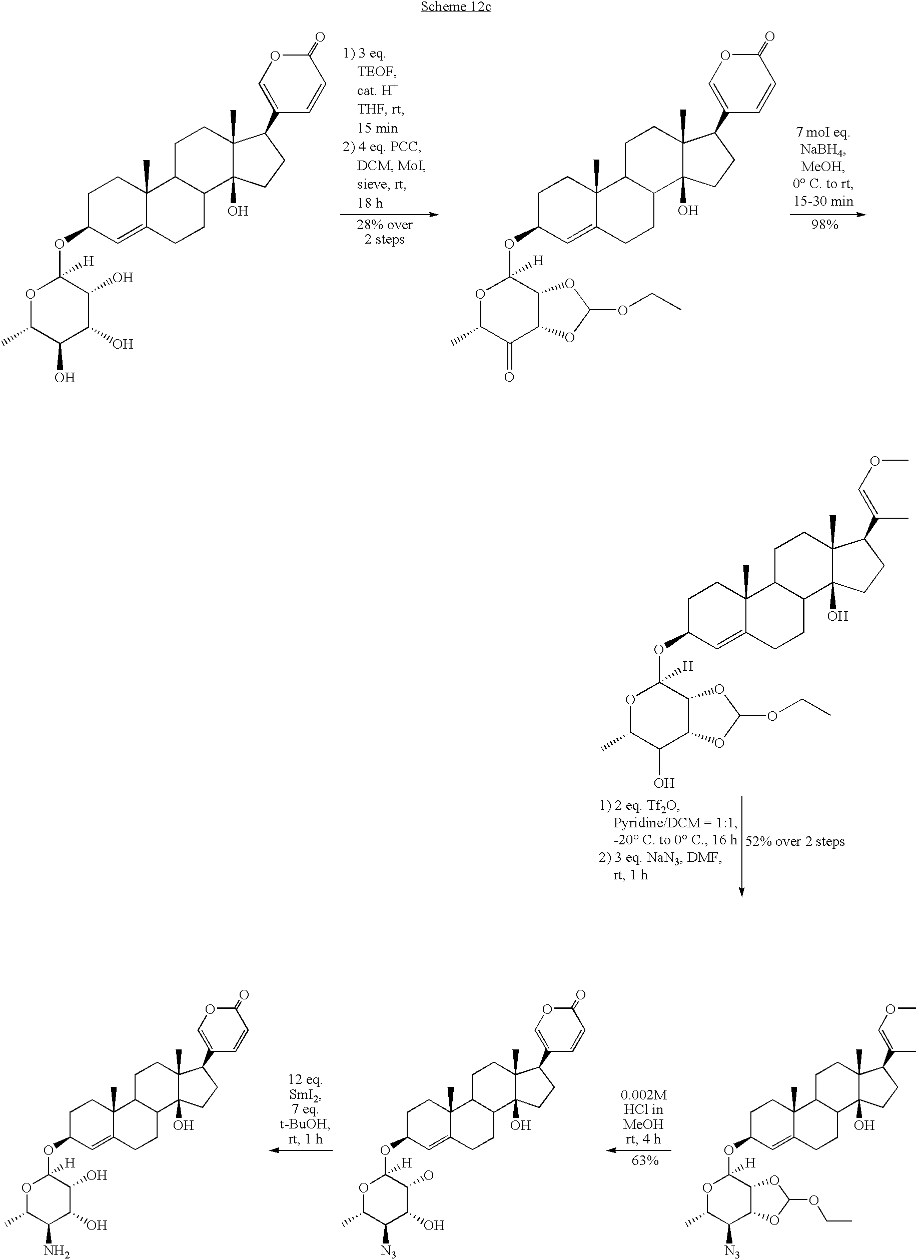

### Example 9

### Preparation of 4,5-Cyclopropyl Derivatives

4,5-Cyclopropyl derivatives can be prepared as shown in Scheme 13.

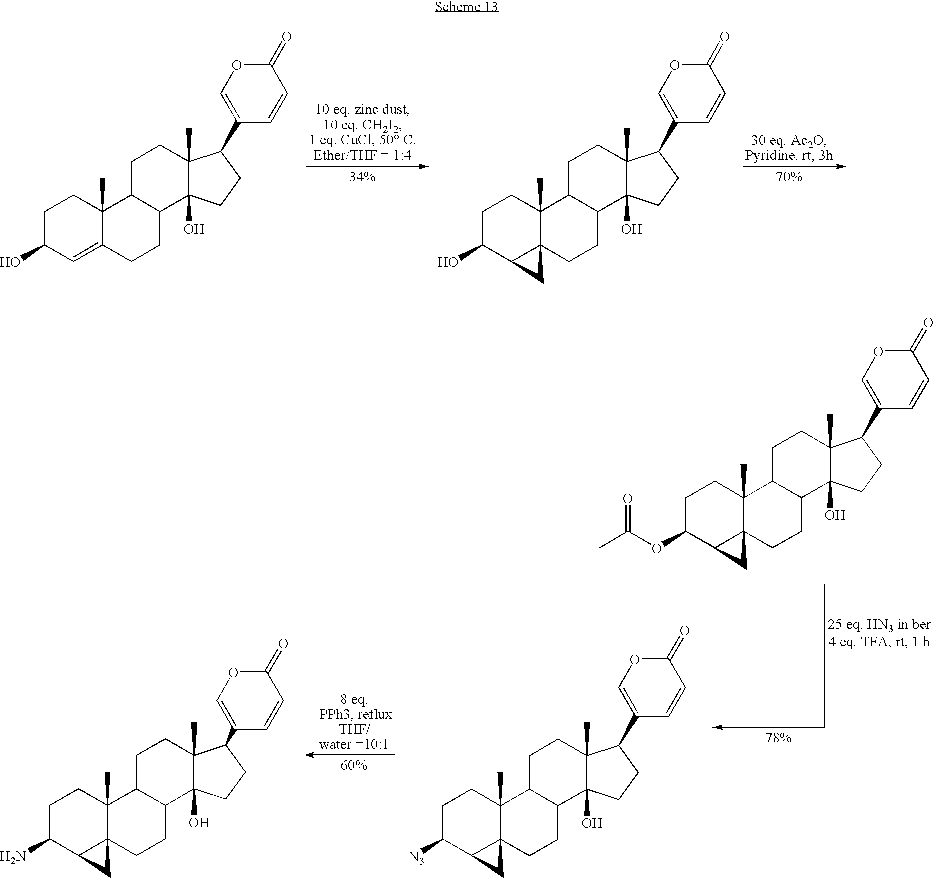

### Example 10

### Broad Spectrum Activity of BNC4 and Novel Analogs BP228 and BP244 Against Human Cancer Cell Lines

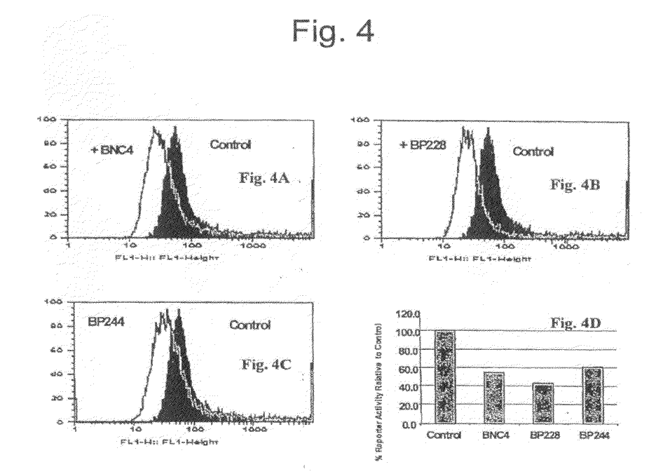

By using the HIF-1α sensitive A549 sentinel line, the cell line was incubated with either BNC4, BP228 or BP244 for 24 hours and reporter activity was measured by FACS analysis. The results are shown in FIG. 4. All three compounds were active in inhibiting the reporter activity (left shift in the FACS curves) and modulating the hypoxia pathway in the cell line.

### Example 11

### BAC4 and Analogs BP228 and BP244 Inhibit Reporter Activity in A549 Sentinel Line

A dose response for each of BP228, BP244, and BNC4 was performed for each cell line and the IC₅₀ value was determined as shown in Table 2. BP244 is the most active compound with an IC₅₀ range of 5-14 nM compared to BNC4 (418 nM) and BP228 (640 nM).

TABLE 2
Anti-Proliferative activity in Tumor Cell Lines

| | | | IC50(nM) | | |
|---|---|---|---|---|---|
| | | | BP228 | BP244 | BNC4 |
| 1 | MCF-7 | Breast (ER+) | 19.8 | 8.2 | 8.4 |
| 2 | DU145 | Prostate (AR-) | 8.8 | 6.7 | 6.2 |
| 3 | LnCaP | Prostate | 39.2 | 13.8 | 16.7 |
| 4 | PC3 | Prostate | 6.2 | 5.7 | 4.1 |
| 5 | MES-SA | Uterine | 11.4 | 8.0 | 8.7 |
| 6 | MES-SA-DX5 | Uterine | 15.8 | 13.5 | 11.6 |
| 7 | HCT116 | Colon | 6.4 | 5.1 | 8.1 |
| 8 | HT29 | Colon | 18.9 | 8.2 | 8.9 |
| 9 | CAKI | Renal | 13.0 | 8.0 | 7.5 |
| 10 | 786-O | Renal | 8.9 | 8.0 | 8.4 |
| 11 | A549 | NSCL | 7.3 | 4.8 | 3.5 |
| 12 | HOP-18 | NSCL | 18.9 | 7.3 | 9.2 |
| 13 | IGR-OV1 | Ovarian | 31.9 | 12.1 | 12.3 |
| 14 | RPMI-8226 | Myeloma | 25.5 | 10.7 | 18.2 |
| 15 | CCRF-CEM | Leukemia | 7.0 | 4.7 | 6.3 |
| 16 | P388 | Leukemia | >1000 | >1000 | >1000 |
| 17 | SNB-75 | CNS | 19.2 | 12.9 | 16.8 |
| 18 | SNB-78 | CNS | 15.9 | 7.7 | 10.1 |
| 19 | C33A | Cervical | 7.2 | 5.1 | 13.6 |
| 20 | PANC | Pancreatic | 8.1 | 6.6 | 3.8 |

### Example 12

### BP228 and BP244 Inhibit Induction of HIF-1α and HIF-2α during Hypoxia

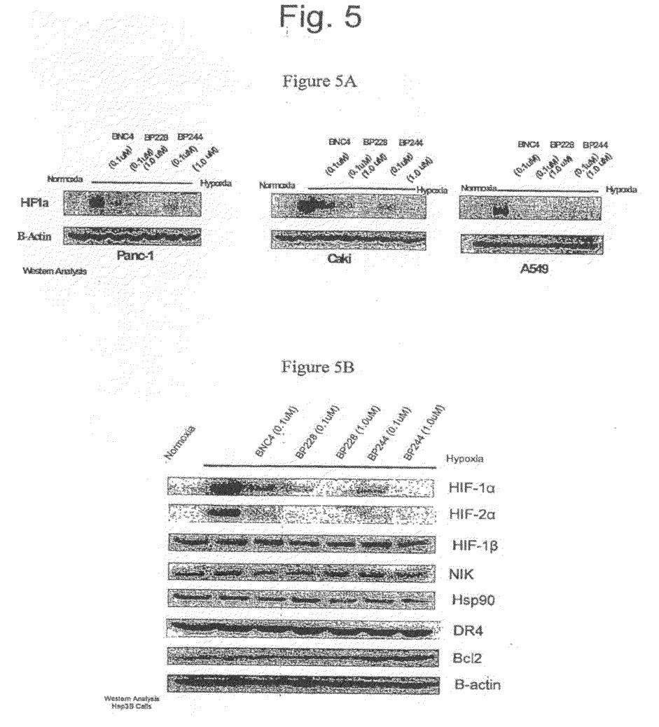

Caki-1 (renal cancer), A549 (lung cancer), Panc-1 (pancreatic cancer) and Hep3B (liver cancer) cells were treated with BNC4, BP228 and BP244 under hypoxic conditions. The cells were treated with indicated each compound for 4 hours under normoxic (N, 20% O₂) or hypoxic (H, 1% O₂) conditions. Expression of HIF-1α, HIF-1β and β-actin and other proteins was analyzed by Western blotting. The HIF-1α and HIF-2α protein levels increased in cells cultured under these conditions for 4 hours without any treatment. Cells treated with BNC4 (at concentrations of 0.1 μM) and BP228 and BP244 at (at 0.1 and 1.0 μM), showed almost complete inhibition of HIF-1α and HIF-2α protein expression (see FIG. 5). The inhibition was specific as levels of constitutively expressed HIF-1β were not affected by any of the drugs. FIG. 5 shows that BNC4, BP244, BP228 compounds specifically inhibit HIF-1α and HIF-2α but had no effect on protein expression of HIF-1β, NIK, Hsp90, DR4, Bcl-2 and β-actin. These results indicate that the compounds are specific and do not inhibit general protein synthesis.

### Example 13

### BNC4, BP244 and BP228 Attenuate Hypoxia Induced VEGF Secretion

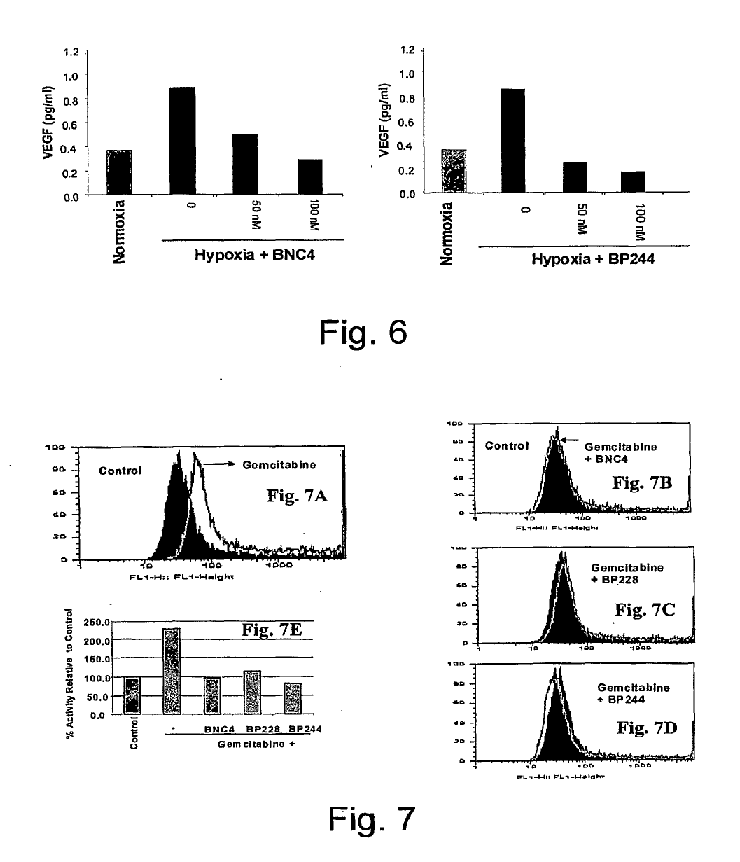

BNC4 and BP244 were shown to reduce VEGF secretion in Hep3B under hypoxic conditions as shown in FIG. 6. The decrease in HIF-1 correlated closely with declining levels of VEGF secretion. Inhibition of VEGF secretion was also demonstrated in A549 (NSCLC) cancer cells. Caki-1 cells were treated with indicated compound and cultured under hypoxia for 16 hours. VEGF levels in conditioned medium were measured using an ELISA kit.

### Example 14

### Inhibition of Hypoxic Stress Response Induced by Cytotoxic Agents

Standard chemotherapeutic agents, such as gemcitabine, were shown to further induce hypoxic response as visualized by A549 sentinel line. Here we show that BNC4, BP228 and BP244 can inhibit the stress response in A549 sentinel line induced by Gemcitabine. Similar results were obtained with carboplatin (not shown).

### Example 15

### Na-K-A TPase Pump and Anti-Proliferative Activity

Na-K-ATPase pump is a heterodimer of alpha and beta subunits. The alpha chain (135 kD)) is the catalytic subunit and contains cation, ATP, and glycoside binding sites. The smaller glycosylated beta subunit (35 kD) is involved primarily in membrane insertion and proper assembly of the functional enzyme. In mammalian cells four different x-isoforms and 3 distinct β-isoforms have been identified. The α1 is expressed in most tissues, while the α2 isoform is predominantly present in skeletal muscle and is also detected in the brain and the heart. The α3 isoform is specifically expressed in neural and cardiac tissues. The β1 and β2 subunits are the predominant isoforms where β1 is ubiquitously expressed and β₂ is limited to neural tissues.

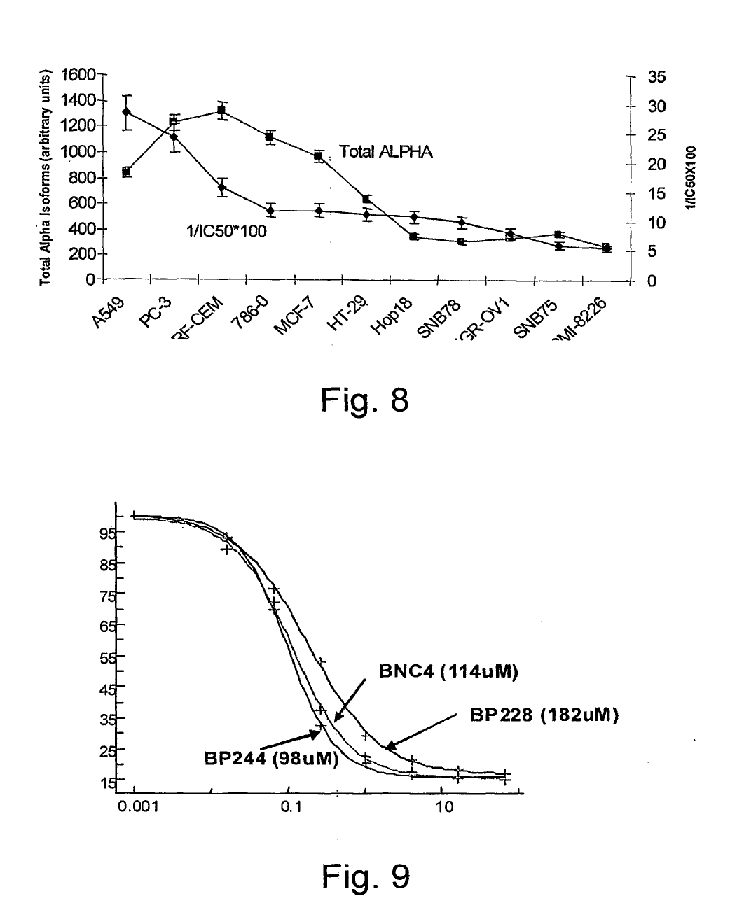

To determine if the anti-proliferative activity BNC4 correlates with the level of Na-K-ATPase in cells the expression of α-1 and α-3 isoforms was measured by real-time RT-PCR (TaqMan) analysis. Alpha subunit is the catalytic domain of Na-K-ATPase. FIG. 8 shows that there is strong correlation between expression levels of alpha (α1+α3) subunits and anti-proliferation activity of BNC4. Cell lines SNB75 (CNS) and RPMI-8226 (leukemia) expressing very low levels of α-chain are very resistant to BNC4 when compared with A549 (Lung cancer) or PC-3 (prostate cancer) cell lines.

### Example 16

### BNC4, BP228 and BP244 Inhibit Activity of Na-K-ATPase, the Physiological Receptor and the Pharmaceutical Target

Compounds were tested for their activity on Na-K-ATPase enzyme in an in vitro enzyme assay. The ATPase activity was assayed as the amount of inorganic phosphate liberated from ATP by Dog Kidney or Porcine cerebral cortex Na-K-ATPase. As shown in FIG. 9, all three compounds inhibit Na-K-ATPase (pig brain) in a dose-dependent manner. Compound BP244 was twice as active as BP228 with an IC₅₀ of 98 μM.

### Example 17

### In Vivo Activity Against Renal Cancer Cell Line Caki-1

Female nude mice (nu/nu) between 5 and 6 weeks of age weighing approximately 20 g were implanted subcutaneously (s.c.) by trocar with fragments of human tumors harvested from s.c. grown tumors in nude mice hosts. When the tumors were approximately 60-75 mg in size (about 10-15 days following inoculation), the animals were pair-matched into treatment and control groups. Each group contained 8-10 mice. The administration of drugs or controls began on the day the animals were pair-matched (Day 1). Pumps (Alzet® Model 2002) with a flow rate of 0.5 μl/hr were implanted s.c. between the shoulder blades of each mice. Mice were weighed and tumor measurements were obtained using calipers twice weekly, starting Day 1. These tumor measurements were converted to mg tumor weight by standard formula, (W²XL)/2. The experiment was terminated when the control group tumor size reached an average of about 1 gram. Upon termination, the mice were weighed, sacrificed and their tumors excised. The tumors were weighed and the mean tumor weight per group was calculated. The change in mean treated tumor weight/the change in mean control tumor weight×100 (dT/dC) was subtracted from 100% to give the tumor growth inhibition (TGI) for each group. Treatment of Caki-1 bearing nude mice with BP244 at 15 mg/ml resulted in 83% tumor growth inhibition (see FIG. 10). The data show that BP244 significantly reduced Caki-1 tumor growth rate without any adverse effects.

### Example 18

### In Vivo Activity of BP244 in Combination with Gemcitabine in Pancreatic Cancer

Panc-1 tumors were injected subcutaneously (sc) into the flanks of male nude mice. After the tumors reached 60 mg in size, osmotic pumps (model 2002, Alzet Inc., flow rate 0.5 μl/hr) containing 15 mg/ml of BP244 were implanted sc on the opposite sides of the mice. The control animals received pumps containing vehicle (10% captisol, Cyclex Inc.). The mice treated with standard chemotherapy agent received intra-peritoneal injections of Gemcitabine at 40 mg/kg every 3 days for 4 treatments (q3d×4). The experiment was terminated when the control group tumor size reached an average of about 1 gram. Upon termination, the mice were weighed, sacrificed and their tumors excised. The tumors were weighed and the mean tumor weight per group was calculated. The change in mean treated tumor weight/the change in mean control tumor weight×100 (dT/dC) was subtracted from 100% to give the tumor growth inhibition (TGI) for each group.

A titration experiment was first performed on BP244 to determine its minimum effective dose against Panc-1 human pancreatic xenograft in nude mice. BP244 (sc, osmotic pumps) was first tested at 15, 10 and 5 mg/ml using Alzet pumps as in previous experiments. Gemcitabine (40 mg/kg; q3d×4, i.p.) was also included in the experiment as a comparison. As shown in FIG. 11A, BP244 at 15 mg/ml was equivalent to 10 mg/ml with TGI of almost 100%. At 5 mg/ml, BP244 (TGI 71%) was as effective as Gemcitabine (TOGI 65%).

A combination study was performed using BP244 and Gemcitabine (FIG. 11B). BP244 at 5 mg/ml was used for the combination study. Combination therapy using both Gemcitabine and BP244 produces a combination effect (TGI 94%), such that sub-optimal doses of both Gemcitabine (40 mg/kg) and BP244, when used together, produce the maximal effect only achieved by higher doses of individual agents alone. There were no deaths in any of the groups and the average weight loss was less than 10%.

Overall BNC4, BP244 and BP228 demonstrated impressive single agent and combination anti tumor activity against Panc-1 model. The data are summarized in Table 3, below.

TABLE 3
Single agent and combination and tumor activity

| | | | % Wt | | Av. Tumor | | |
|---|---|---|---|---|---|---|---|
| | | | Change | | Weight | | % TGI |
| Group | n | Dose/Route | (d24) | % SD | (mg) | SD | (d24) |
| Vehicle Control | 8 | Captisol; SC; CI | 5.77 | 2.5 | 1101.4 | 239.9 | 0 |
| Gemcitabine | 8 | 40 mg/kg: IV: q3d × 4 | 2.60 | 1.9 | 414.3 | 105.1 | 65 |
| BNC4 | 8 | 15 mg/ml; SC; CI | −2.69 | 2.8 | 243.9 | 45.5 | 87 |
| Gem + BNC4 | 8 | ″ | 10.95 | 1.9 | 87.9 | 102.0 | 99 |
| BP228 (10) | 8 | 10 mg/ml; SC; CI | 1.97 | 1.9 | 488.0 | 38.7 | 66 |
| BP228 (15) | 8 | 15 mg/ml; SC; CI | 4.88 | 3.1 | 327.0 | 91.9 | 79 |
| Gem + BP228 (10) | 8 | ″ | −2.42 | 3.3 | 140.5 | 12.7 | 93 |
| BP244 (5) | 8 | 5 mg/ml; SC; CI | −4.63 | 2.9 | 524.4 | 10.0 | 71 |
| BP244 (10) | 8 | 10 mg/ml; SC; CI | 0.93 | 2 | 107.3 | 16.8 | 98 |
| BP244 (15) | 8 | 15 mg/ml; SC; CI | 5.26 | 2 | 44.2 | 38.4 | 102 |
| Gem + BP244 (5) | 8 | ″ | −1.24 | 1.8 | 146.6 | 25.6 | 94 |
| Gem + BP244 (10) | 8 | ″ | −4.12 | 1.7 | 71.3 | 13.6 | 99 |

### Example 19

### In Vitro Data for 3-Esters

In vitro data for 3-ester derivatives are provided in Table 4. “AICAR-RA” refers to the reporter assay (RA) on the AMP analogue 5-aminoimidazole-4-carbox-amide riboside (AICAR), which is indicative of the inhibition of glucose metabolism.

TABLE 4

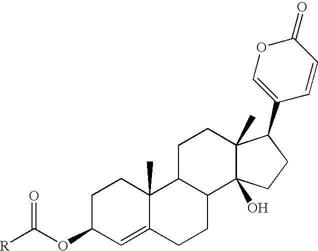

| | APA | APA | ATPase | ATPase | | | APA |
|---|---|---|---|---|---|---|---|
| | (A549) | (Caki-1) | Inh, IC₅₀ | Inh, IC₅₀ | AICAR- | AHA | (Panc-1) |
| | IC₅₀ | IC₅₀ | (nM) | (nM) | RA ED₅₀ | (+ − | IC₅₀ |
| R | (nM) | (nM) | Dog kidney | Pig brain | (nM) | ++++) | (nM) |
| 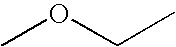 | 11 | 141 | 220 | 250 | 111 | ++++ | |
| 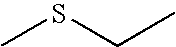 | 14 | 202 | 180 | 312 | 112 | +++ | |
| 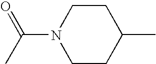 | 15 | 342 | 270 | 396 | 133 (ave) | +++ | |
| 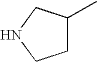 | 48 | 112 | 299 | 480 | 210 | ++ | |
| 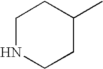 | 20 | 120 | | 136 | 101 | ++++ | |
| 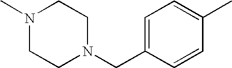 | 16 | 107 | | | 100 | | |
| 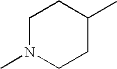 | 31 | 103 | | | 100 | | |
| 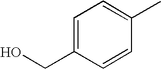 | 27 | 105 | | | 110 | | |
| 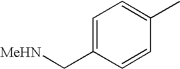 | 26 | 113 | | | 110 | | |
| 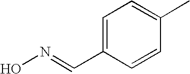 | 30 | 68 | | | 110 | | |
| 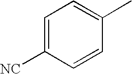 | 55 | 84 | | 453 | 130 | | |

### Example 20

### In Vitro Data for 3-Carbamates

In vitro data for 3-carbamate derivatives are provided in Table 5.

TABLE 5

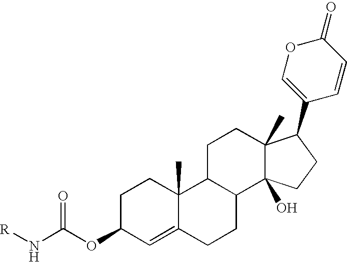

| | APA | APA | ATPase | ATPase | | | APA |
|---|---|---|---|---|---|---|---|
| | (A549) | (Caki-1) | Inh, IC₅₀ | Inh, IC₅₀ | AICAR- | AHA | (Panc-1) |
| | IC₅₀ | IC₅₀ | (nM) | (nM) | RA ED₅₀ | (+ − | IC₅₀ |
| R | (nM) | (nM) | Dog kidney | Pig brain | (nM) | ++++) | (nM) |
| 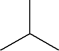 | 29 | 197 | 279 | 273 | 112 | ++ | |
| 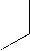 | 35 | 82 | 220 | 210 | 75 (ave) | +++ | |
| 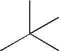 | 45 | 276 | | 207 | 104 | +++ | |
| 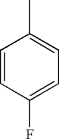 | 23 | 142 | | 277 | 100 | +++ | |
| 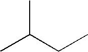 | 28 | 84 | | 304 | 90 | ++++ | |
| 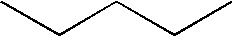 | 24 (ave) | 46 (ave) | | 206 | 68 (ave) | ++++ | |
| 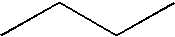 | 19 | 67 | | 198 | 102 | ++++ | |
| 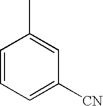 | 40 | 100 | | | 94 | | |
| 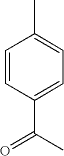 | 33 | 242 | | | 110 | | |
| 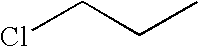 | 33 | 78 | | | 95 | | |
| 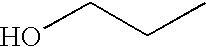 | 24 | 92 | | | | | |
| 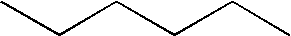 | | 50 | | | | | |
| 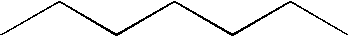 | 1810 | 8498 | | 820 | | | 4449 |

### Example 21

### In Vitro Data for 3-Oximethers

In vitro data for 3-oximether derivatives are provided in Table 6.

TABLE 6

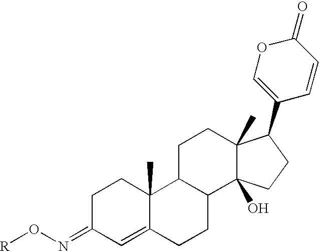

| | APA | APA | ATPase | ATPase | | | APA |
|---|---|---|---|---|---|---|---|
| | (A549) | (Caki-1) | Inh, IC₅₀ | Inh, IC₅₀ | AICAR- | AHA | (Panc-1) |
| | IC₅₀ | IC₅₀ | (nM) | (nM) | RA ED₅₀ | (+ − | IC₅₀ |
| R | (nM) | (nM) | Dog kidney | Pig brain | (nM) | ++++) | (nM) |
| 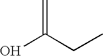 | 92 | 600 (ave) | | 1005 | 405 (ave) | +++ | |
| 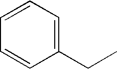 | 202 | 263 | | | 100 | | |
| 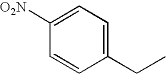 | 681 | 984 | | | 1680 | | |
| 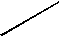 | 38 (ave) | 41 (ave) | | 460 (ave) | 62 | | |
| 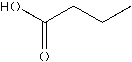 | 156 | 399 | | | 466 | | |
| 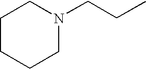 | 7 (ave) | 27 (ave) | | 164 (ave) | | | 16 |
| 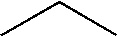 | 14 | 19 | | 93 | | | |
| 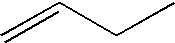 | 9 | 40 | | 116 | | | |
| 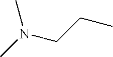 | 2 | 24 | | 85 | | | |

### Example 22

### In Vitro Data for Miscellaneous Compounds

In vitro data for compounds of the invention are provided in Table 7.

TABLE 7

| | APA | APA | ATPase | ATPase | | | APA |
|---|---|---|---|---|---|---|---|
| | (A549) | (Caki-1) | Inh, IC₅₀ | Inh, IC₅₀ | AICAR- | AHA | (Panc-1) |
| | IC₅₀ | IC₅₀ | (nM) | (nM) | RA ED₅₀ | (+ − | IC₅₀ |
| R | (nM) | (nM) | Dog kidney | Pig brain | (nM) | ++++) | (nM) |
| 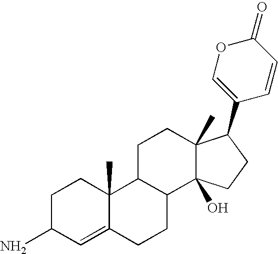 | 5 (ave) | 72 | | 93 | | | |
| 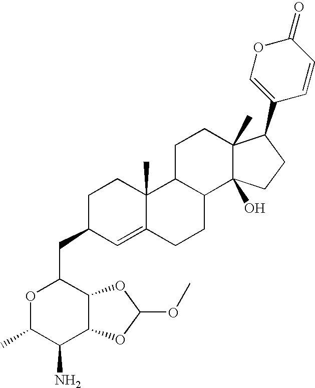 | 3 | 12 | | 206 | | | 11 |
| 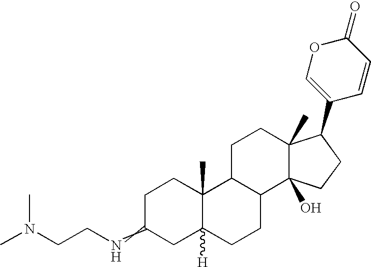 | 25 | 109 | | 171 | | | |
| 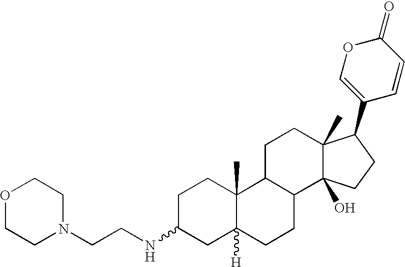 | 101 | 276 | | 356 | | | |
| 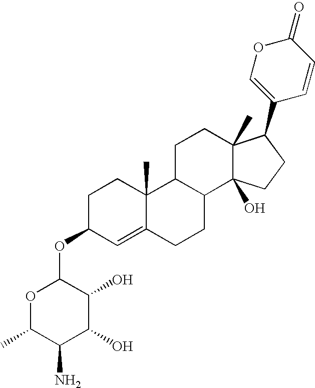 | 7 | 56 | | 540 | | | 21 |
| 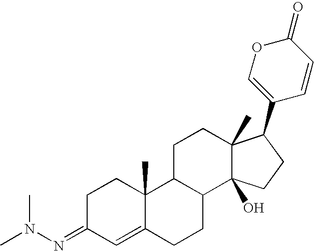 | 23 | 118 | | 196 | 102 | ++++ | |
| 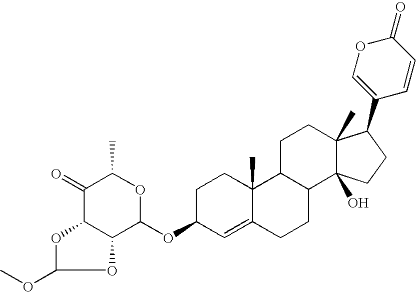 | | 26 | | 169 | | | |
| 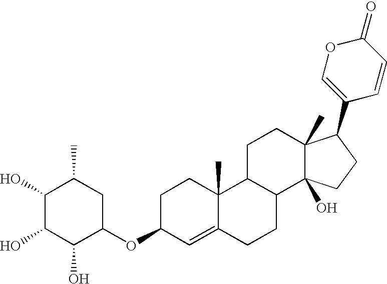 | 9 | 24 | | 129 | | | |

### Example 23

### Pharmacokinetics Following IP Administration in Mice

The pharmacokinetic profiled of BNC4, BP228 and BP244 in mice is provided in FIG. 13. The compounds were administered by intraperitoneal (i.p) injection at 2.5 mg/kg and 5.0 mg/kg for BP228 and at 5.0 mg/kg for BNC4 and BP244. The plasma samples were collected at various time points and concentration of compounds was analyzed by LC-MS.

Mean concentration-time profiles for serum BNC228 following intraperitoneal administration at 2.5 and 5 mg/kg were similar, with concentrations attaining maximal values at 10 minutes (0.167 hours; tₘₐₓ) and 5 minutes (0.083 hours) postdose, respectively, and then declining in an apparent multi-phasic manner. Mean concentrations were measurable through 6 hours (tₗₐₛₜ) at both dosages, and apparent terminal elimination half-lives were similar, 1.5 hours at 2.5 mg/kg and 1.9 hours at 5 mg/kg.

The mean concentration-time profile for serum BP244 at a dosage of 5 mg/kg was characterized by an increase in concentration to Cₘₐₓ at 30 minutes (0.5 hours; tₘₐₓ) postdose and then a general decline through 24 hours (tₗₐₛₜ), with a terminal last elimination half-life estimate of 4.5 hours.

Mean concentrations of serum BNC4, after dosing at 5 mg/kg, increased to near the maximal level by the first sampling time (5 minutes) and were sustained at that approximate level through 30 minutes postdose, with Cₘₐₓ observed at 15 minutes (0.25 hours; tₘₐₓ). Concentrations then declined through the 6-hour sampling time (tₗₐₛₜ), with a terminal elimination half-life estimate of 0.80 hours.

Cₘₐₓ for serum BP228 increased in an approximate dosage proportional manner from 715 ng/mL at 2.5 mg/kg to 1200 ng/mL at 5 mg/kg. Cₘₐₓ for BP244 and BNC4, each administered at 5 mg/kg, was 2120 ng/mL and 3610 ng/mL, respectively.

AUC for serum BP228 also increased in an apparent dosage proportional manner from 1020 ng·h/mL at 2.5 mg/lkg to 2350 ng·h/mL at 5 mg/kg. The AUC for BP244 and BNC4, each administered at 5 mg/kg, was 4630 ng·h/mL and 4570 ng·h/mL, respectively.

The pharmacokinetic data are summarized in Table 8, below.

TABLE 8

| | BNC228 | BNC228 | BNC244 | BNC4 |
|---|---|---|---|---|
| Parameter | 2.5 mg/kg | 5 mg/kg | 5 mg/kg | 5 mg/kg |
| Cₘₐₓ (ng/mL) | 715 | 1200 | 2120 | 3610 |
| tₘₐₓ (h) | 0.167 | 0.0833 | 0.5 | 0.25 |
| tₗₐₛₜ (h) | 6 | 6 | 24 | 6 |
| AUC (ng × h/mL) | 1020 | 2350 | 4630 | 4570 |
| t_{1/2} (h) | 1.5 | 1.9 | 4.5 | 0.8 |

### Other Embodiments

All publications, patents, and patent applications mentioned in this specification are herein incorporated by reference to the same extent as if each independent publication or patent application was specifically and individually indicated to be incorporated by reference.

While the invention has been described in connection with specific embodiments thereof, it will be understood ihat it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

1. A compound of formulas I or II: 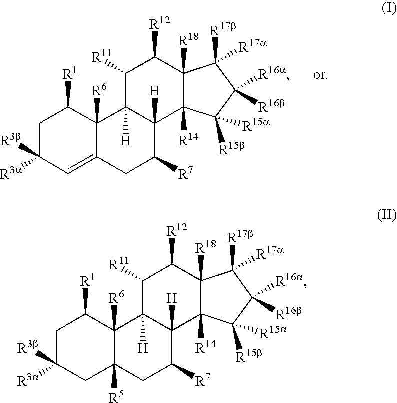
or a pharmaceutically acceptable salt or prodrug thereof, wherein
each of R¹, R⁵, R⁷, R¹¹, and R¹² is, independently, H; OH, OR^{1A}, or OC(O)R^{1A}, where R^{1A} is C₁₋₇ alkyl, C₂₋₇ alkenyl, C₂₋₇ alkynyl, C₂₋₆ heterocyclyl, C₁₋₂ aryl, C₇₋₁₄ alkaryl, C₃₋₁₀ alkheterocyclyl, or C₁₋₇ heteroalkyl; or
each of R³ and R³³ is, independently, H, OC(O)NHR^{3C}, OC(O)NR^{3D}R^{3E}, NH₂, NHR^{3F}, NR^{3G}R^{3H}, NHC(O)R^{3I}, NHC(O)OR^{3J}, NR^{3K}C(O)OR^{3L}, or NH-Sac, where each of R^{3C}, R^{3D}, R^{3E}, R^{3F}, R^{3G}, R^{3H}, R^{3I}, R^{3J}, R^{3K}, and R^{3L} is, independently, C₁₋₇ alkyl, C₂₋₇ alkenyl, C₂₋₇ alkynyl, C₂₋₄ heterocyclyl, C₆₋₁₂ aryl, C₇₋₁₄ alkaryl, C₃₋₁₀ alkheterocyclyl, or C₁₋₇ heteroalkyl, and Sac is a saccharide; or
each of R^{3α} and R^{3β} is, independently, H, OR^{3A} or OC(O)R^{3B} and each of R^{3A} and R^{3B} is, independently, C₂₋₄ heterocyclyl, C₆₋₁₂ aryl, C₇₋₁₄ alkaryl, C₃₋₁₀ alkheterocyclyl, or C₁₋₇ heteroalkyl, with the proviso that at least one of R^{3α} and R^{3β} is not H; or
R^{3α} and R^{3β} together are ═NNR^{3M}R^{3N}, or ═NOR^{3P}, wherein each of R^{3M}, R^{3N} and R^{3P} is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, and with the proviso that at least one of R^{3α} and R^{3β} is not H;
R⁶ is CH₃, CH₂OR^{6A}, or CH₂OCOR^{6A}, where R^{6A} is H, C₁₋₇ alkyl, C₂₋₇ alkenyl, C₂₋₇ alkynyl, C₂— heterocyclyl, C₆₋₁₂ aryl, C₇₋₁₄ alkaryl, C₃₋₁₀ alkheterocyclyl, or C₁₋₇ heteroalkyl;
R¹⁴ is OH, Cl, OR^{14A}, or OC(O)R^{14A}, where R^{14A} is C₁₋₇ alkyl, C₂₋₇ alkenyl, C₂₋₇ alkynyl, C₂₋₄ heterocyclyl, C₆₋₁₂ aryl, C₇₋₁₄ alkaryl, C₃₋₁₀ alkheterocyclyl, or C₁₋₇ heteroalkyl, or R¹⁴, R^{15β}, and the carbons they are bonded to together represent an epoxide;
each of R^{15α} and R^{15β} is, independently, H, OH, OR^{15A}, or OC(O)R^{15A}, where R^{15A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{15α} and R^{15β} together are ═O;
each of R^{16α} and R^{16β} is, independently, H, OH, OR^{16A}, or OC(O)R^{16A}, where R^{16A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{16α} and R^{16β} together are ═O;
R^{17β} is 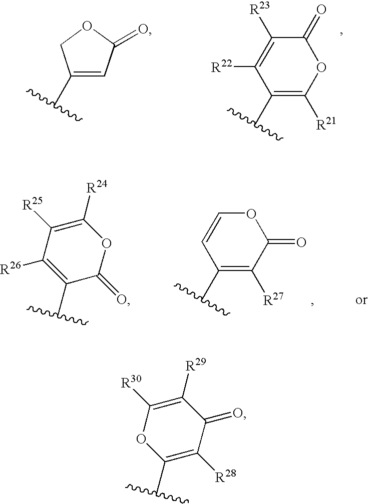
where each of R²¹, R²², R²³, R²⁴, R²⁵, R²⁶, R²⁷, R²⁸, R²⁹, and R³⁰ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl;
R^{17α}, is H or OH; and
R¹⁸ is CH₃, CH₂OR^{18A}, or CH₂OCOR^{18A}, where R^{18A} is H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

2. A compound of formulas Ia or IIa: 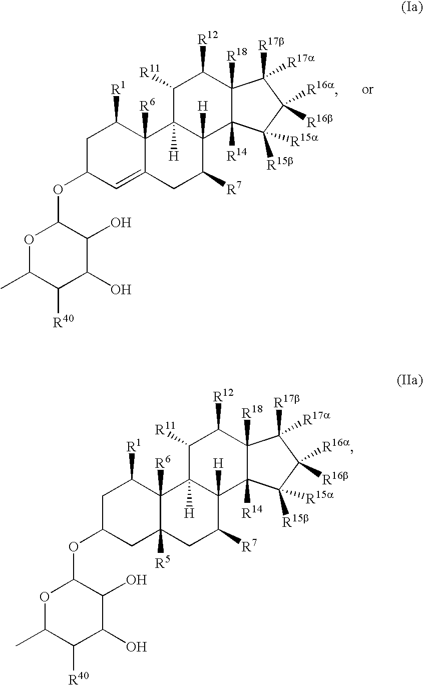
or a pharmaceutically acceptable salt or prodrug thereof, wherein
each of R¹, R⁵, R⁷, R¹¹, and R¹² is, independently, H; OH, OR^{1A}, or OC(O)R^{1A}, where R^{1A} is C₁₋₇ alkyl, C₂₋₇ alkenyl, C₂₋₇ alkynyl, C₂₋₆ heterocyclyl, C₁₋₁₂ aryl, C₇₋₁₄ alkaryl, C₃₋₁₀ alkheterocyclyl, or C₁₋₇ heteroalkyl;
R⁶ is CH₃, CH₂OR^{6A}, or CH₂OCOR^{6A}, where R^{6A} is H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl;
R¹⁴ is OH, Cl, OR^{14A}, or OC(O)R^{14A}, where R^{14A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R¹⁴, R^{15β}, and the carbons they are bonded to together represent an epoxide;
each of R^{15α} and R^{15β} is, independently, H, OH, OR^{15A}, or OC(O)R^{15A}, where R^{15A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{15α} and R^{15β} together are ═O;
each of R^{16α} and R^{16β} is, independently, H, OH, OR^{16A}, or OC(O)R^{16A}, where R^{16A} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{16α} and R^{16β} together are ═O;
R^{17β} is 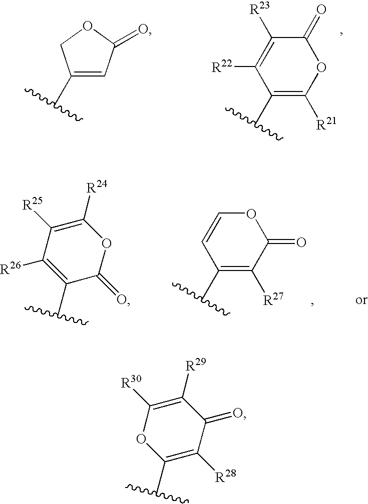
where each of R²¹, R²², R²³, R²⁴, R²⁵, R²⁶, R²⁷, R²⁸, R²⁹, and R³⁰ is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl;
R^{17α}, is H or OH;
R¹⁸ is CH₃, CH₂OR^{18A}, or CH₂OCOR^{18A}, where R^{18A} is H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; and
R⁴⁰ is F, Cl, CF₃, NH₂, NHR^{40A}, NR^{41B}R^{40C} NHC(O)R^{40D}, NHC(S)R^{40E}, NHC(O)OR^{4F}, NHC(S)OR^{40G}, NHC(O)NHR^{40H}, NHC(S)NHR^{40I}, NHC(O)SR^{40J}, NHC(S)SR^{40K}, or NHS(O)₂R^{40L}, and where each of R^{40A}, R^{40B}, R^{40C}, R^{40D}, R^{40E}, R^{40F}, R^{40G}, R^{40H}, R^{40I}, R^{40J}, R^{40K} and R^{40L} is, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl; or R^{40B} and R^{40C} combine to form a C₂ heterocyclyl containing at least one nitrogen atom.

3. The compound of claim 1 or 2, wherein each of R¹, R^{3α}, R⁵, R⁷, R¹¹, R¹², R^{15α}, R^{15β}, R^{16α}, and R^{16β} is H.

4. The compound of claim 1 or 2, wherein each of R⁶ and R¹⁸ is CH₃.

5. The compound of claim 1 or 2, wherein R¹⁴ is OH.

6. The compound of claim 1 or 2, wherein R^{3β} is OC(O)NHR^{3C}, OC(O)NR^{3D}R^{3E}, NH₂, NHR^{3F}, NR^{3G}R^{3H}, NHC(O)R^{3I}, NHC(O)OR^{3J}, NR KC(O)OR^{3K}, or NH-Sac.

7. The compound of claim 1 or 2, wherein R^{17β} is

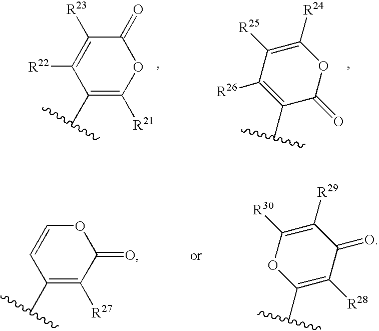

8. The compound of claim 7, wherein R^{17β} is

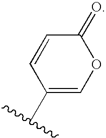

9. The compound of claim 8, wherein R^{3β} is NH-Sac; Sac is described by the formula: 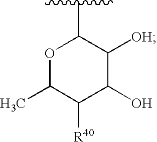
wherein R⁴⁰ is F, Cl, CF₃, OH, NH₂, NHR^{40A}, NR^{40B}R^{40C}, NHC(O)R^{40D}, NHC(S)R^{40E}, NHC(O)OR^{40F}, NHC(S)OR^{40G}, NHC(O)NHR^{40H}, NHC(S)NHR^{40I}, NHC(O)SR^{40J}, NHC(S)SR^{40K}, or NHS(O)₂R^{40L}; and each of R^{40A}, R^{40B}, R^{40C}, R^{40D}, R^{40E}, R^{40F}, R^{40G}, R^{40H}, R^{40I}, R^{40J}, R^{40K}, and R^{40L} is, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{40B} and R^{40C} combine to form a C₂— heterocyclyl containing at least one nitrogen atom.

10. The compound of claim 9, wherein said compound is

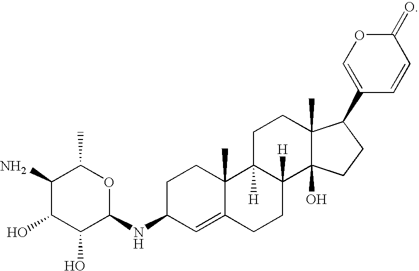

11. The compound of claim 1, wherein said compound is

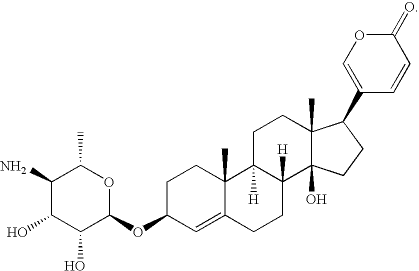

12. The compound of claim 1, wherein R^{3α} and R^{3β} together are ═NNR^{3M}R^{3N}, or ═NOR^{3P}, wherein each of R^{3M}, R^{3N} and R^{3P} is, independently, H, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

13. The compound of claim 12, wherein R^{3α} and R^{3β} together are ═NOR^{3P}, wherein R^{3P} is C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl.

14. The compound of claim 13, wherein said compound is

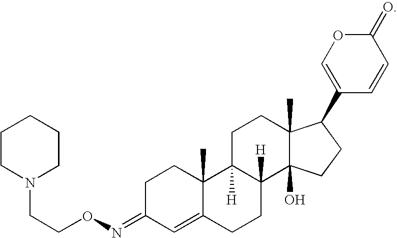

15. A method for treating a disorder in a mammal mediated by hypoxia inducible factor-1 (HIF-1), said method comprising administering to said mammal a compound of claims 1 or 2, in an amount sufficient to treat said disorder.

16. The method of claim 15, wherein said disorder is characterized by pathogenic angiogenesis.

17. The method of claim 16, wherein said disorder is an ocular disorder.

18. The method of claim 17, wherein said ocular disorder is optic disc neovascularization, iris neovascularization, retinal neovascularization, choroidal neovascularization, corneal neovascularization, vitreal neovascularization, glaucoma, pannus, pterygium, macular edema, diabetic macular edema, vascular retinopathy, retinal degeneration, uveitis, inflammatory diseases of the retina, excessive angiogenesis following cataract surgery, or proliferative vitreoretinopathy.

19. The method of claim 18, wherein said disorder is a neoplastic disorder.

20. The method of claim 19, wherein said neoplastic disorder is carcinoma of the bladder, breast, colon, kidney, liver, lung, head and neck, gall-bladder, ovary, pancreas, stomach, cervix, thyroid, prostate, or skin; a hematopoietic cancer of lymphoid lineage; a hematopoietic cancer of myeloid lineage; a cancer of mesenchymal origin; a cancer of the central or peripheral nervous system; melanoma; seminoma; teratocarcinoma; osteosarcoma; thyroid follicular cancer; or Kaposi's sarcoma.

21. A method for reducing VEGF expression in a cell, said method comprising contacting said cell with a compound of claims 1 or 2, in an amount sufficient to reduce said VEGF expression.

22. A method for treating a patient with a neoplastic disorder, said method comprising administering to said patient (i) a compound of claims 1 or 2, and (ii) an antiproliferative agent, wherein said compound, and said antiproliferative agent are administered simultaneously, or within 14 days of each other, each in an amount that together is sufficient to treat said neoplastic disorder.

23. The method of claim 22, wherein said antiproliferative agent is selected from alkylating agents, folic acid antagonists, pyrimidine antagonists, purine antagonists, antimitotic agents, DNA topomerase II inhibitors, DNA topomerase I inhibitors, taxanes, DNA intercalators, aromatase inhibitors, 5-alpha-reductase inhibitors, estrogen inhibitors, androgen inhibitors, gonadotropin releasing hormone agonists, retinoic acid derivatives, and hypoxia selective cytotoxins.

24. The method of claim 23, wherein said antiproliferative agent is gemcitabine.

25. A kit comprising:
(i) a compound of claims 1 or 2; and
(ii) instructions for administering said compound to a patient diagnosed with a disorder mediated by hypoxia inducible factor-1 (HIF-1).

26. The kit of claim 25, further comprising an antiproliferative agent.

27. The kit of claim 26, wherein said compound and said antiproliferative agent are formulated together for simultaneous administration.

28. A method for synthesizing a compound of claim 1, wherein R^{3α}, and R^{3β} together are ═NOR^{3P}, said method comprising the step of condensing H₂NOR^{3P} with a 3-oxo cardiolide or 3-oxo bufadienolide, wherein R^{3P} is H, C₁₋₇ alkyl, C₂₋₇ alkenyl, C₂₋₇ alkynyl, C₂— heterocyclyl, C₆₋₁₂ aryl, C₇₋₁₄ alkaryl, C₃₋₁₀ alkheterocyclyl, or C₁₋₇ heteroalkyl.

29. A method for synthesizing a compound of claim 2, wherein R^{3α} or R^{3β} is O-β-amino-Sac from the corresponding azide wherein R^{3α} or R^{3β} is O-β-azido-Sac, said method comprising the step of reducing said corresponding azide to form an amine, wherein β-azido-Sac is described by formula s1 and β-amino-Sac is described by formula s2:

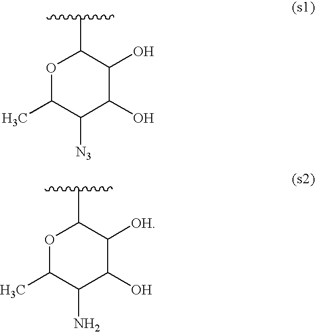

30. A method for synthesizing a compound of claim 1 or 2, wherein R^{3α} or R^{3β} is O-Sac or NH-Sac, said method comprising the step of condensing HO-Sac with a cardiolide or bufadienolide, wherein Sac is described by the formula: 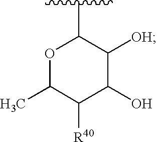
wherein R⁴⁰ is F, Cl, CF₃, OH, NH₂, NHR^{40A}, NR^{41B}, R^{40C}, NHC(O)R^{40D}, NHC(S)R^{40E}, NHC(O)OR^{40F}, NHC(S)OR^{40G}, NHC(O)NHR^{40H}, NHC(S)NHR^{40I}, NHC(O)SR^{40J}, NHC(S)SR^{40K}, or NHS(O)₂R^{40L}; and each of R^{40A}, R^{40B}, R^{40C}, R^{40D}, R^{40E}, R^{40F}, R^{40G}, R^{40H}, R^{40I}, R^{40J}, R^{40K} and R^{40L} is, independently, C₁₋₇ alkyl, C₂₋₇ alkenyl, C₂₋₇ alkynyl, C₂₋₆ heterocyclyl, C₆₋₁₂ aryl, C₇₋₁₄ alkaryl, C₃₋₁₀ alkheterocyclyl, or C₁₋₇ heteroalkyl, or R^{40B} and R^{40C} combine to form a C₂₋₆ heterocyclyl containing at least one nitrogen atom.
